(19) United States
(12) Patent Application Publication
(10) Pub. No.: US 2025/0382378 A1
(43) Pub. Date: Dec. 18, 2025

(54) CD38 ANTIBODIES AND USES THEREOF

(71) Applicant: GENMAB A/S, Valby (DK)

(72) Inventors: Tahamtan AHMADI, Plainsboro, NJ (US); Sieto BOSGRA, Utrecht (NL); Esther C.W. BREIJ, Utrecht (NL); Jenny J. CHEN, Plainsboro, NJ (US); Ida HIEMSTRA, Utrecht (NL); Kate SASSER, Doylestown, PA (US); Claudia Silvia CORRADO, Copenhagen V (DK); Xu Steven XU, Plainsboro, NJ (US)

(21) Appl. No.: 19/125,633

(22) PCT Filed: Oct. 30, 2023
(86) PCT No.: PCT/EP2023/080292

§ 371 Date: Apr. 29, 2025

Related U.S. Application Data
(60) Provisional application No. 63/421,009, filed on Oct. 31, 2022.

(51) Int. Cl. C07K 16/28; A61K 9/00; A61K 38/19; A61K 39/00; A61K 47/22; A61K 47/26; A61P 35/00; A61P 37/06

(52) U.S. Cl. CPC C07K 16/2896; A61K 9/0019; A61K 38/193; A61K 47/22; A61K 47/26; A61P 35/00; A61P 37/06; A61K 2039/505; A61K 2039/54; A61K 2039/545; C07K 2317/31; C07K 2317/35; C07K 2317/526; C07K 2317/565; C07K 2317/732; C07K 2317/734

(57) ABSTRACT

The present invention relates to anti-CD38 antibodies comprising one or more mutations in the Fc region, and the use of such antibodies in the treatment of diseases in subjects, such as hematological malignancies.

### FIELD OF THE INVENTION

The present invention relates to anti-CD38 antibodies comprising one or more mutations in the Fc region, and the use of such antibodies in the treatment of diseases in subjects.

### BACKGROUND OF THE INVENTION

CD38 is a type II transmembrane glycoprotein which is normally found on hematopoietic cells and at low levels in certain solid tissues. Expression of CD38 in hematopoietic cells depends on the differentiation and activation status of the cell. Lineage-committed hematopoietic cells express the protein, while it is lost by mature cells and expressed again on activated lymphocytes. CD38 is also expressed on B cells, whereby plasma cells express particularly high levels of CD38. Approximately 80% of resting NK cells and monocytes express CD38 at lower levels, as do various other hematological cell types, including lymph node germinal center lymphoblasts, intrafollicular cells, dendritic cells, erythrocytes, and platelets (Lee and Aarhus 1993; Zocchi, Franco et al. 1993; Malavasi, Funaro et al. 1994; Ramaschi, Torti et al. 1996). With regard to solid tissues, CD38 is expressed in the gut by intraepithelial cells and lamina propria lymphocytes, by Purkinje cells and neurofibrillary tangles in the brain, by epithelial cells in the prostate, β-cells in the pancreas, osteoclasts in the bone, retinal cells in the eye, and sarcolemma of smooth and striated muscle.

CD38 is expressed in a large number of hematological malignancies. Expression has been observed particularly in the malignant cells of multiple myeloma (MM) (Lin, Owens et al. 2004) and chronic lymphocytic leukemia (CLL) (Damle 1999), and was also reported in Waldenström's macroglobulinemia (Konoplev, Medeiros et al. 2005), primary systemic amyloidosis (Perfetti, Bellotti et al. 1994), mantle-cell lymphoma (Parry-Jones, Matutes et al. 2007), acute lymphoblastic leukemia (Keyhani, Huh et al. 2000), acute myeloid leukemia (Marinov, Koubek et al. 1993; Keyhani, Huh et al. 2000), NK-cell leukemia (Suzuki, Suzumiya et al. 2004), NK/T-cell lymphoma (Wang, Wang et al. 2015) and plasma cell leukemia (van de Donk, Lokhorst et al. 2012).

Other diseases, where CD38 expression could be involved, include, e.g. broncho-epithelial carcinomas of the lung, breast cancer (evolving from malignant proliferation of epithelial lining in ducts and lobules of the breast), pancreatic tumors, evolving from the β-cells (insulinomas), tumors evolving from epithelium in the gut (e.g. adenocarcinoma and squamous cell carcinoma), carcinoma in the prostate gland, seminomas in testis, ovarian cancers, and neuroblastomas. Other disclosures also suggest a role of CD38 in autoimmunity such as Graves disease and thyroiditis (Antonelli, Fallahi et al. 2001), type 1 and 2 Diabetes (Mallone and Perin 2006) and inflammation of airway smooth muscle cells during asthma (Deshpande, White et al. 2005). Moreover, CD38 expression has been associated with HIV infection (Kestens, Vanham et al. 1992; Ho, Hultin et al. 1993).

CD38 is a multifunctional protein. Functions ascribed to CD38 include both receptor mediation in adhesion and signaling events and (ecto-) enzymatic activity. As an ectoenzyme, CD38 uses NAD⁺ as substrate for the formation of cyclic ADP-ribose (cADPR) and ADPR, but also of nicotinamide and nicotinic acid-adenine dinucleotide phosphate (NAADP). cADPR has been shown to act as second messenger for Ca²⁺ mobilization from the endoplasmatic reticulum.

Several anti-CD38 antibodies are described in the literature, for instance in WO 2006/099875 A1, WO2008037257 A2, WO 2011/154453 A1, WO 2007/042309 A1, WO 2008/047242 A1, WO2012/092612 A1, Cotner, Hemler et al. 1981; Ausiello, Urbani et al. 2000; Lande, Urbani et al. 2002; de Weers, Tai et al. 2011; Deckert, Wetzel et al. 2014; Raab, Goldschmidt et al. 2015; Eissler, Filosto et al. 2018; Roepcke, Plock et al. 2018; and Schooten 2018.

CD38 antibodies may affect CD38 expressing tumor cells by one or more of the following mechanisms of action: complement-dependent cytotoxicity (CDC), antibody-dependent cellular cytotoxicity (ADCC), antibody-dependent cellular phagocytosis (ADCP), programmed cell death, trogocytosis, elimination of immune suppressor cells and modulation of enzymatic activity (van de Donk, Janmaat et al. 2016; Krejcik, Casneuf et al. 2016; Krejcik, Frerichs et al. 2017; Chatterjee, Daenthanasanmak et al. 2018; van de Donk 2018). However, in 2014, it was proposed that, no CD38 antibodies had been described that could induce effective CDC, ADCC, ADCP as well as effectively inhibit CD38 enzyme activity (Lammerts van Bueren, Jakobs et al. 2014).

Optimization of the effector functions may improve the effectivity of therapeutic antibodies for treating cancer or other diseases, e.g., to improve the ability of an antibody to elicit an immune response to antigen-expressing cells. Such efforts are described in, e.g., WO 2013/004842 A2; WO 2014/108198 A1; WO 2018/031258 A1; Dall′Acqua, Cook et al. 2006; Moore, Chen et al. 2010; Desjarlais and Lazar 2011; Kaneko and Niwa 2011; Song, Myojo et al. 2014; Brezski and Georgiou 2016; Sondermann and Szymkowski 2016; Zhang, Armstrong et al. 2017; Wang, Mathieu et al. 2018.

WO 2020/012036, WO 2020/012038 and WO 2021/144457 describe advantegeous anti-CD38 antibody variants with modulated potencies, as well as uses and formulations thereof (all incorporated by reference herein).

Although there are currently treatment regimens available for the treatment of CD38+ cancers, such as MM, there is still a need for further therapeutic options, as there still remain patients that relapse or are refractory to currently available treatments.

Hence, one object of the present invention is to provide for means and methods for treating a cancer by using an anti-CD38 antibody as described herein, as well as providing an anti-CD38 antibody as described herein, for use in the treatment of a cancer, more specifically hematological cancers such as MM.

Specific doses, ranges and/or dosage regimens are provided that are advantageous with regard to such methods or uses, such as for a cancer known or identified as being positive for CD38, such as a MM. More in particular, specific doses, ranges and/or dosage regimens are provided herein that are advantageous with regard to the treatment of patients suffering from (relapsed or refractory) MM. The dose ranges and/or dosage regimens provided herein were assessed to be safe for human use and/or shown to be effective in the treatment of MM.

### SUMMARY OF THE INVENTION

The present invention concerns anti-CD38 antibodies, particularly of antibody C, having one or more mutations in the Fc region, and uses thereof in the treatment of hematological malignancies such as MM. At least one of these mutations is in a residue corresponding to E430, E345 or S440 in a human IgG1 heavy chain, wherein the amino acid residues are numbered according to the EU index.

So, in one aspect, the invention relates to a method of treating or preventing a hematological malignancy, preferably a multiple myeloma (MM), in a subject in need thereof, preferably a human subject, comprising administering to said subject, an antibody binding to human CD38 in a therapeutically effective amount, said antibody comprising:
a. an antigen-binding region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4, a VL CDR1 having the sequence as set forth in SEQ ID NO:6, a VL CDR2 having the sequence AAS, and a VL CDR3 having the sequence as set forth in SEQ ID NO:7, and
b. an Fc region comprising a mutation in one or more amino acid residues selected from the group corresponding to E430, E345 and S440 in a human IgG1 heavy chain, wherein the amino acid residues are numbered according to the EU index.

In another aspect, the invention relates to a method of treating or preventing a hematological malignancy, preferably a multiple myeloma (MM), in a subject in need thereof, preferably a human subject, comprising administering to said subject, an antibody or a pharmaceutical composition comprising the antibody in a therapeutically effective amount, said antibody comprising:
a. a heavy chain comprising a VH region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4 and a human IgG1 CH region with a mutation in one or more of E430, E345 and S440, the amino acid residues being numbered according to the EU index; and
b. a light chain comprising a VL region comprising a VL CDR1 having the sequence as set forth in SEQ ID NO:6, a VL CDR2 having the sequence AAS, and a VL CDR3 having the sequence as set forth in SEQ ID NO:7

In another aspect, the invention relates to a method of treating or preventing a hematological malignancy, preferably a multiple myeloma (MM), in a subject in need thereof, preferably a human subject, comprising administering to said subject, an antibody binding to human CD38 in a therapeutically effective amount, said antibody comprising:
(a) a heavy chain comprising a VH region comprising SEQ ID NO:1 and a human IgG1 CH region with a mutation in one or more of E430, E345 and S440, wherein the amino acid residue numbering is according to the EU index, and
(b) alight chain comprising a VL comprising SEQ ID NO:5.

In one embodiment, the mutation comprises or consists of a mutation at position E430, preferably E430G.

In one embodiment, the antibody is administered at a dose of at least (about) 4 mg/kg body weight, such as between (about_4 mg/kg to (about) 24 mg/kg body weight.

In one embodiment, the antibody is administered at a dose of (about) 8 mg/kg to (about) 16 mg/kg body weight.

In one embodiment, the antibody is administered at a dose of (about) 16 mg/kg body weight.

In one embodiment, the antibody is administered in cycles of 28 days (4 weeks), with a weekly administration in cycles 1 and 2 (Q1W), a biweekly administration in cycles 3 through 6 (Q2W), and a monthly administration (Q4W) as of cycle 7, wherein the first dose is a split dose.

In one embodiment, the hematological malignancy is a cancer that is refractory to a prior therapy, such as a prior therapy comprising an anti-CD38 antibody, e.g. daratumumab or isatuximab.

In one embodiment, the hematological malignancy is (relapsed or refractory) multiple myeloma or (relapsed or refractory) DLBCL.

In one embodiment, the administration of said antibody induces one or more therapeutic effects (relative to a baseline). In another embodiment, the administration of said antibody improves one or more therapeutic effects (relative to a baseline).

Said one or more therapeutic effects may be overall response rate, duration of response, time to response.

Said one or more therapeutic effects may be a stringent complete response, complete response, very good partial response, partial response, minimal response or stable disease status.

In one embodiment, said one or more therapeutic effects is improved compared to a reference antibody.

In one embodiment, said subject may be treated for the management of neutropenia or infusion related reactions.

In one embodiment, wherein said subject displays a faster clearance of said antibody compared to a reference antibody or the antibody displays a faster clearance compared to a reference antibody.

In one embodiment, administration of said antibody may have one or more of the following effects:
a. activation of the complement system in said subject;
b. depletion of peripheral blood NK cells in said subject;
c. expansion of peripheral blood T cells in said subject.

In one embodiment, induction of complement activation preferably is greater compared to a reference antibody.

In one embodiment, administration of said antibody does not result in a (substantial), dose dependent increase in plasma levels of proinflammatory cytokines.

In one embodiment, administration of said antibody does not induce a dose-dependent reduction in B cells, T cells, monocytes and/or NKT-like cells.

In one embodiment, the reference antibody does not comprise a mutation in one or more amino acid residues selected from the group corresponding to E430, E345 and S440, preferably the reference antibody is an IgG1 antibody, such as a wt IgG1 antibody. The reference antibody may be daratumumab or isatuximab

In one embodiment, the antibody is comprised in a composition comprising:
a) said antibody, optionally in a concentration of 1 to 200 mg/mL
b) 5-40 mM histidine or acetate;
c) 100-400 mM sorbitol or sucrose; and
d) a surfactant.

In one embodiment, the antibody is comprised in a composition comprising having a pH of about 6 and comprising, consisting or consisting essentially of:
a) about 20 mg/mL of the antibody,
b) about 20 mM histidine,
c) about 250 mM sorbitol, and
d) about 0.04% w/v of polysorbate 80,
optionally in aqueous solution.

In one aspect, the invention relates to the anti-CD38 antibody or a composition comprising said antibody according to any aspect or embodiment herein for use in treating or preventing a hematological malignancy involving cells expressing CD38 as described herein.

In one aspect, the invention relates to the anti-CD38 antibody or a composition comprising said antibody according to any aspect or embodiment herein for use in treating or preventing a hematological malignancy in a subject comprising cells expressing human CD38 as described herein.

In one aspect, the invention provides the anti-CD38 antibody or a composition comprising said antibody according to any of the embodiments and aspects as described herein, for use in the prevention or treatment of a hematological malignancy as described herein.

In one aspect, the invention relates to the anti-CD38 antibody or a composition comprising said antibody according to any aspect or embodiment herein for use as a medicament for treating or preventing a hematological malignancy as described herein.

In one aspect, the invention provides the anti-CD38 antibody or a composition comprising said antibody according to any of the embodiments and aspects as described herein, for the manufacture of a medicament for the prevention or treatment of a hematological malignancy as described herein.

These and other aspect and embodiments of the invention are described in more detail below.

### LEGENDS TO THE FIGURES

FIG. 1 shows an amino acid sequence alignment using Clustal 2.1 software for human IgG1m(a), IgG1 m(f), IgG2, IgG3 and IgG4 Fc segments corresponding to residues P247 to K447 in the human IgG1 heavy chains, wherein the amino acid residues are numbered according to the EU index as set forth in Kabat. The amino acid sequences shown correspond to residues 130 to 330 in the heavy chain constant regions of the allotypic variants of human IgG1 designated IgG1m(za) (SEQ ID NO:64; UniProt accession No. P01857), IgG1m(f) (SEQ ID NO:65), IgG1m(z) (SEQ ID NO:66), IgG1m(a) (SEQ ID NO:67) and IgG1m(x) (SEQ ID NO:68); residues 126 to 326 of the IgG2 heavy chain constant region (SEQ ID NO:79; UniProt accession No. P01859); residues 177 to 377 of the IgG3 heavy chain constant region (SEQ ID NO:80; UniProt accession No. P01860), and residues 127 to 327 of the IgG4 heavy chain constant region (SEQ ID NO:81; UniProt accession No. P01861).

FIG. 2 shows a schematic overview of the IgG-C-E430G trial design. DL=dose level; IA=interim analysis; IV=intravenous; MTD=maximum tolerated dose; RP2D=recommended phase 2 dose; RRMM=relapsed or refractory multiple myeloma; R/R DLBCL=relapsed or refractory diffuse large B-cell lymphoma.

FIG. 3 shows absolute NK cell numbers (CD3−/CD56+/CD16+ cells/μL) in peripheral blood of RRMM patients dosed with IgG1-C-E430G during the dose escalation part of the IgG-C-E430G trial. NK cell counts are shown over time and grouped per dose cohort (0.2/0.6 mg/kg, 2 mg/kg, 4 mg/kg, 8 mg/kg, 16 mg/kg, and 24 mg/kg). The horizontal dashed line marked with * indicates 5 cells/μL, vertical dotted lines at C1D1, C1D8, C1D15, and C1D21 illustrate administration of IgG1-C-E430G.

FIG. 4 shows relative change of NK cell numbers (% CD3−/CD56+/CD16+ cells) from NK cell count on C1D1 in peripheral blood of RRMM patients dosed with IgG1-C-E430G during the dose escalation part of the IgG-C-E430G trial. Relative change in NK cell numbers are shown over time and grouped per dose cohort (0.2/0.6 mg/kg, 2 mg/kg, 4 mg/kg, 8 mg/kg, 16 mg/kg, and 24 mg/kg). The horizontal dashed line marked with * indicates relative NK cell numbers on C1D1 (baseline level), vertical dotted lines at C1 D1, C1 D8, C1D15, and C1 D21 illustrate administration of IgG1-C-E430G.

FIG. 5 shows absolute T cell numbers (CD3+ cells/μL) in peripheral blood of RRMM patients dosed with IgG1-C-E430G during the dose escalation part of the IgG-C-E430G trial. T cell counts are shown over time and grouped per dose cohort (0.2/0.6 mg/kg, 2 mg/kg, 4 mg/kg, 8 mg/kg, 16 mg/kg, and 24 mg/kg). The horizontal dashed line marked with * indicates 5 cells/μL, vertical dotted lines at C1 D1, C1D8, C1D15, and C1D21 illustrate administration of IgG1-C-E430G.

FIG. 6 shows relative change of T cell numbers (% CD3+ cells) from T cell count on C1 D1 in peripheral blood of RRMM patients dosed with IgG1-C-E430G during the dose escalation part of the IgG-C-E430G trial. Relative change in T cell numbers are shown over time and grouped per dose cohort (0.2/0.6 mg/kg, 2 mg/kg, 4 mg/kg, 8 mg/kg, 16 mg/kg, and 24 mg/kg). The horizontal dashed line marked with * indicates relative T cell numbers on C1 D1 (baseline level), vertical dotted lines at C1 D1, C1D8, C1D15, and C1D21 illustrate administration of IgG1-C-E430G.

FIG. 7 shows absolute cytokine concentrations in peripheral blood of RRMM patients dosed with IgG1-C-E430G during the dose escalation part of the IgG-C-E430G trial. Levels of Interferon gamma (IFN gamma), Interleukin 2 (IL-2), Interleukin 6 (IL-6), Interleukin 8 (IL-8), Interleukin 10 (IL-10), and Tumor Necrosis Factor Alpha (TNF-alpha) are shown overtime and grouped per dose cohort (0.2/0.6 mg/kg, 2 mg/kg, 4 mg/kg, 8 mg/kg, 16 mg/kg, and 24 mg/kg). Vertical dotted lines at C1D1, C1D8, C1 D15, and C1D21 illustrate administration of IgG1-C-E430G. Subjects 0016, 0018 and 0024 had values outside the scale for parameter IL-8 and subject 0019 had values outside the scale for parameter INF-Gamma.

FIG. 8 shows a diagram of the bioanalytical method. An anti-ideotype antibody (dark gray—lower antibody) was used as a coating reagent to capture IgG1-C-E430G (middle gray—middle antibody). The captured drug was detected with a second, sulfo-tagged anti-ideotype antibody (light gray—upper antibody). The signal was quantified by electrochemoluminescence.

FIG. 9 shows the serum concentrations of IgG1-C-E430G (μg/mL) plotted against time (days) on semi-log scale, grouped by dose level. Symbols represent individual observations of serum concentrations; lines connect the observations in each patient. Actual time of sample collection relative to the pretreatment sample was used. Vertical dashed lines mark the scheduled time points of drug administration. The horizontal line marks the lower limit of quantification of the bioanalytical assay of 0.05 μg/mL. Observations below the lower limit of quantification were plotted at half the limit of quantification (0.025 μg/mL).

FIG. 10 shows the AUC₀₋ₜ (d*μg/mL) and CL (L/d/kg) against dose (mg/kg) on log-log scale calculated if feasible for each patient from PK profiles collected around the Cycle 1 Day 1/Day2, Cycle 1 Day 8/Day 9 (patient E only) and Cycle 2 Day 1 administrations. Symbols and error bars represent means and standard deviations per dose and PK profile.

FIG. 11 shows absolute and relative CD4+ T cell numbers (CD3+/CD4+ cells) in peripheral blood of RRMM patients dosed with IgG1-C-E430G during the dose escalation part of the first-in-human trial (data cut off: 14 Aug. 2023). Panel A shows absolute CD4+ T cell numbers (CD3+/CD4+ cells/μL). Panel B shows relative change of CD4+ T cell numbers (% CD3+/CD4+ cells) compared to CD4+ T cell count on C1 D1 in peripheral blood. CD4+ T cell counts and relative change in CD4+ T cell numbers are shown over time and grouped per dose cohort (0.2/0.6 mg/kg, 2 mg/kg, 4 mg/kg, 8 mg/kg, 16 mg/kg, and 24 mg/kg). The horizontal dashed line in panel B indicates 50% increase of CD3+CD4+ cells compared to baseline. The maximum value on the y-axis in panel B has been set to 150%. Values may exist above this limit.

FIG. 12 shows absolute and relative CD8+ T cell numbers (CD3+/CD8+ cells) in peripheral blood of RRMM patients dosed with IgG1-C-E430G during the dose escalation part of the first-in-human trial (data cut off: 14 Aug. 2023). Panel A shows absolute CD8+ T cell numbers (CD3+/CD8+ cells/μL). Panel B shows relative change of CD8+ T cell numbers (% CD3+/CD8+ cells) compared to CD8+ T cell count on C1D1 in peripheral blood. CD8+ T cell counts and relative change in CD8+ T cell numbers are shown over time and grouped per dose cohort (0.2/0.6 mg/kg, 2 mg/kg, 4 mg/kg, 8 mg/kg, 16 mg/kg, and 24 mg/kg). The horizontal dashed line in panel B indicates 50% increase of CD3+CD8+ cells compared to baseline. The maximum value on the y-axis in panel B has been set to 150%. Values may exist above this limit.

FIG. 13 shows absolute and relative NK cell numbers (CD3−/CD56+/CD16+) in peripheral blood of RRMM patients dosed with 16 mg/kg IgG1-C-E430G during expansion part A of the first-in-human trial over time. Panel A shows absolute NK cell numbers (CD3−/CD56+/CD16+ cells/μL). Panel B shows relative change of NK cell numbers (% CD3−/CD56+/CD16+ cells) compared to NK cell count on C1D1 in peripheral blood. The maximum value on the y-axis in panel B has been set to 150%. Values may exist above this limit.

FIG. 14 shows absolute and relative CD4+ T cell numbers (CD3+/CD4+ cells) in peripheral blood of RRMM patients dosed with 16 mg/kg IgG1-C-E430G during the expansion part A of the first-in-human trial over time (data cut off: 14 Aug. 2023). Panel A shows absolute CD4+ T cell numbers (CD3+/CD4+ cells/μL). Panel B shows relative change of CD4+ T cell numbers (% CD3+/CD4+ cells) compared to CD4+ T cell count on C1 D1 in peripheral blood. The horizontal dashed line in panel B indicates 50% increase of CD3+CD4+ cells compared to baseline. The maximum value on the y-axis in panel B has been set to 150%. Values may exist above this limit.

FIG. 15 shows absolute and relative CD8+ T cell numbers (CD3+/CD8+ cells) in peripheral blood of RRMM patients dosed with 16 mg/kg IgG1-C-E430G during the expansion part A of the first-in-human trial over time (data cut off: 14 Aug. 2023). Panel A shows absolute CD8+ T cell numbers (CD3+/CD8+ cells/μL). Panel B shows relative change of CD8+ T cell numbers (% CD3+/CD8+ cells) compared to CD8+ T cell count on C1 D1 in peripheral blood. The horizontal dashed line in panel B indicates 50% increase of CD3+CD8+ cells compared to baseline. The maximum value on the y-axis in panel B has been set to 150%. Values may exist above this limit.

FIG. 16 shows absolute cytokine concentrations in peripheral blood of RRMM patients dosed with 16 mg/kg IgG1-C-E430G during the expansion part A of the first-in-human trial (data cut off: 14 Aug. 2023). Levels of Interferon gamma (IFN gamma), Interleukin 10 (IL-10), Interleukin 2 (IL-2), Interleukin 6 (IL-6), Interleukin 8 (IL-8), and Tumor Necrosis Factor Alpha (TNF-alpha) are shown over time.

FIG. 17 shows comparison of serum concentrations of IgG1-C-E430G in RRMM patients dosed with 16/mg dose during the dose escalation (A) and RRMM patients dosed with 16 mg/kg during the expansion part A (B) of the first in human trial.

### DETAILED DESCRIPTION OF THE INVENTION

In describing the embodiments of the invention specific terminology will be resorted to for the sake of clarity. However, the invention is not intended to be limited to the specific terms so selected, and it is understood that each specific term includes all technical equivalents which operate in a similar manner to accomplish a similar purpose.

### Definitions

As used herein, the term “CD38” generally refers to human CD38 (UniProtKB-P28907 (CD38_HUMAN)) having the sequence set forth in SEQ ID NO:38, but may also, unless contradicted by context, refer to variants, isoforms and orthologs thereof. Variants of human CD38 with S274, Q272R, T237A or D202G mutations are described in WO 2006/099875 A1 and WO 2011/154453 A1.

The term “immunoglobulin” refers to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable (VH) region and a heavy chain constant (CH) region. The CH region typically is comprised of three domains, CH1, CH2, and CH3. The heavy chains are typically inter-connected via disulfide bonds in the so-called “hinge region”. Each light chain typically is comprised of a light chain variable (VL) region and a light chain constant region, the latter typically comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL region is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901 917 (1987)).

Unless otherwise stated or contradicted by context, CDR sequences herein are identified according to IMGT rules using DomainGapAlign (Lefranc M P., Nucleic Acids Research 1999; 27:209-212 and Ehrenmann F., Kaas Q. and Lefranc M.-P.

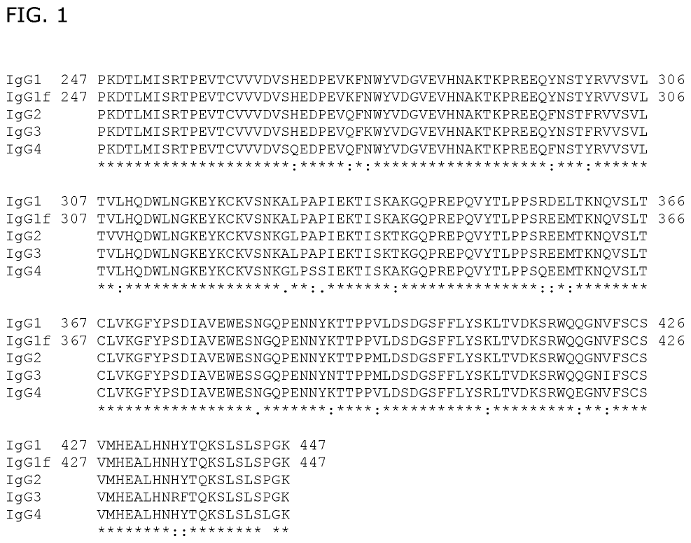

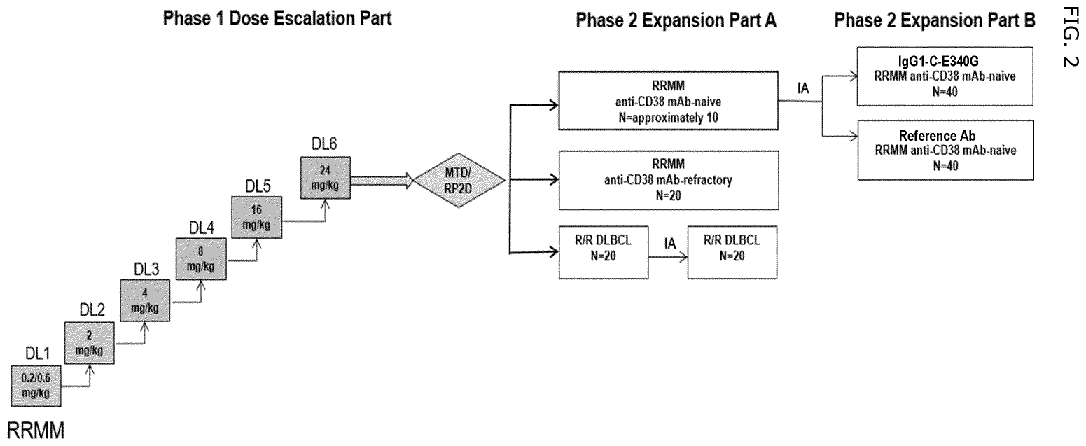

Nucleic Acids Res., 38, D301-307 (2010); see also internet http address www.imgt.org/. Unless otherwise stated or contradicted by context, reference to amino acid positions in the CH or Fc region/Fc domain in the present invention is according to the EU-numbering (Edelman et al., Proc Natl Acad Sci USA. 1969 May; 63(1):78-85; Kabat et al., Sequences of proteins of immunological interest. 5th Edition—1991 NIH Publication No. 91-3242). An amino acid residue in a CH of another isotype than human IgG1 may, however, alternatively be referred to by the corresponding amino acid position in a wild-type human IgG1 heavy chain in which the amino acid residues are numbered according to the EU index. Specifically, the corresponding amino acid position can be identified as illustrated in FIG. 1, i.e., by (a) aligning the amino acid sequence of the non-IgG1 constant region (or a segment thereof) with the amino acid sequence of a human IgG1 heavy chain (or segment thereof) in which the amino acid residues are numbered according to the EU index, and (b) identifying which amino acid position in the IgG1 heavy chain the amino acid residue is aligned with. Accordingly, the position of such an amino acid residue can herein be referred to as “the amino acid residue at a position corresponding to”, followed by the amino acid position in a wild-type human IgG1 heavy chain numbered according to the EU index. When referring to one or more of a number of different amino acid positions, this can be referred to herein as “a mutation in one or more amino acid residues at positions selected from the group consisting of the positions corresponding to”, “a mutation in one or more amino acid residues at positions corresponding to” or simply “a mutation in one or more amino acid residues selected from the group corresponding to”, followed by two or more amino acid positions (e.g., E430, E345 and S440) in a human wild-type IgG1 heavy chain, wherein the amino acid residues are numbered according to the EU index.

The term “hinge region” as used herein is intended to refer to the hinge region of an immunoglobulin heavy chain. Thus, for example the hinge region of a human IgG1 antibody corresponds to amino acids 216-230 according to the EU numbering.

The term “CH2 region” or “CH2 domain” as used herein is intended to refer to the CH2 region of an immunoglobulin heavy chain. Thus, for example the CH2 region of a human IgG1 antibody corresponds to amino acids 231-340 according to the EU numbering. However, the CH2 region may also be any of the other subtypes as described herein.

The term “CH3 region” or “CH3 domain” as used herein is intended to refer to the CH3 region of an immunoglobulin heavy chain. Thus, for example the CH3 region of a human IgG1 antibody corresponds to amino acids 341-447 according to the EU numbering. However, the CH3 region may also be any of the other subtypes as described herein.

The term “antibody” (Ab), sometimes also referred to as binding agent, in the context of the present invention refers to an immunoglobulin molecule, a fragment of an immunoglobulin molecule, or a derivative of either thereof, which has the ability to specifically bind to an antigen. The antibody of the present invention comprises an Fc-domain of an immunoglobulin and an antigen-binding region. An antibody generally contains two CH2-CH3 regions and a connecting region, e.g. a hinge region, e.g. at least an Fc-domain. Thus, the antibody of the present invention may comprise an Fc region and an antigen-binding region. The variable regions of the heavy and light chains of the immunoglobulin molecule contain a binding domain that interacts with an antigen. The constant or “Fc” regions of the antibodies may mediate the binding of the immunoglobulin to host tissues or factors, including various cells of the immune system (such as effector cells) and components of the complement system such as C1q, the first component in the classical pathway of complement activation. As used herein, unless contradicted by context, the Fc region of an immunoglobulin typically contains at least a CH2 domain and a CH3 domain of an immunoglobulin CH, and may comprise a connecting region, e.g., a hinge region. An Fc-region is typically in dimerized form via, e.g., disulfide bridges connecting the two hinge regions and/or non-covalent interactions between the two CH3 regions. The dimer may be a homodimer (where the two Fc region monomer amino acid sequences are identical) or a heterodimer (where the two Fc region monomer amino acid sequences differ in one or more amino acids). Preferably, the dimer is a homodimer. An Fc region-fragment of a full-length antibody can, for example, be generated by digestion of the full-length antibody with papain, as is well-known in the art. An antibody as defined herein may, in addition to an Fc region and an antigen-binding region, further comprise one or both of an immunoglobulin CH1 region and a CL region. An antibody may also be a multispecific antibody, such as a bispecific antibody or similar molecule. The term “bispecific antibody” refers to an antibody having specificities for at least two different, typically non-overlapping, epitopes. Such epitopes may be on the same or different targets. If the epitopes are on different targets, such targets may be on the same cell or different cells or cell types. As indicated above, unless otherwise stated or clearly contradicted by the context, the term antibody herein includes fragments of an antibody which comprise at least a portion of an Fc-region and which retain the ability to specifically bind to the antigen. Such fragments may be provided by any known technique, such as enzymatic cleavage, peptide synthesis and recombinant expression techniques. It has been shown that the antigen-binding function of an antibody may be performed by fragments of a full-length antibody. Examples of binding fragments encompassed within the term “Ab” or “antibody” include, without limitation, monovalent antibodies (described in WO2007059782 by Genmab); heavy-chain antibodies, consisting only of two heavy chains and naturally occurring in e.g. camelids (e.g., Hamers-Casterman (1993) Nature 363:446); ThioMabs (Roche, WO2011069104), strand-exchange engineered domain (SEED or Seed-body) which are asymmetric and bispecific antibody-like molecules (Merck, WO2007110205); Triomab (Pharma/Fresenius Biotech, Lindhofer et al. 1995 J Immunol 155:219; WO2002020039); FcAAdp (Regeneron, WO2010151792), Azymetric Scaffold (Zymeworks/Merck, WO2012/058768), mAb-Fv (Xencor, WO2011/028952), Xmab (Xencor), Dual variable domain immunoglobulin (Abbott, DVD-Ig, U.S. Pat. No. 7,612,181); Dual domain double head antibodies (Unilever; Sanofi Aventis, WO20100226923), Di-diabody (ImClone/Eli Lilly), Knobs-into-holes antibody formats (Genentech, WO9850431); DuoBody (Genmab, WO 2011/131746); Bispecific IgG1 and IgG2 (Pfizer/Rinat, WO11143545), DuetMab (MedImmune, US2014/0348839), Electrostatic steering antibody formats (Amgen, EP1870459 and WO 2009089004; Chugai, US201000155133; Oncomed, WO2010129304A2); bispecific IgG1 and IgG2 (Rinat neurosciences Corporation, WO11143545), CrossMAbs (Roche, WO2011117329), LUZ-Y (Genentech), Biclonic (Merus, WO2013157953), Dual Targeting domain antibodies (GSK/Domantis), Two-in-one Antibodies or Dual action Fabs recognizing two targets (Genentech, NovImmune, Adimab), Cross-linked Mabs (Karmanos Cancer Center), covalently fused mAbs (AIMM), CovX-body (CovX/Pfizer), FynomAbs (Covagen/Janssen ilag), DutaMab (Dutalys/Roche), iMab (MedImmune), IgG-like Bispecific (ImClone/Eli Lilly, Shen, J., et al. J Immunol Methods, 2007. 318(1-2): p. 65-74), TIG-body, DIG-body and PIG-body (Pharmabcine), Dual-affinity retargeting molecules (Fc-DART or Ig-DART, by Macrogenics, WO/2008/157379, WO/2010/080538), BEAT (Glenmark), Zybodies (Zyngenia), approaches with common light chain (Crucell/Merus, U.S. Pat. No. 7,262,028) or common heavy chains (klBodies by NovImmune, WO2012023053), as well as fusion proteins comprising a polypeptide sequence fused to an antibody fragment containing an Fc-region like scFv-fusions, like BsAb by ZymoGenetics/BMS, HERCULES by Biogen Idec (U.S. Pat. No. 7,951,918), SCORPIONS by Emergent BioSolutions/Trubion and Zymogenetics/BMS, Ts2Ab (MedImmune/AZ (Dimasi, N., et al. J Mol Biol, 2009. 393(3): p. 672-92), scFv fusion by Genentech/Roche, scFv fusion by Novartis, scFv fusion by Immunomedics, scFv fusion by Changzhou Adam Biotech Inc (CN 102250246), TvAb by Roche (WO 2012025525, WO 2012025530), mAb² by f-Star (WO2008/003116), and dual scFv-fusion s. It should be understood that the term antibody, unless otherwise specified, includes monoclonal antibodies (such as human monoclonal antibodies), polyclonal antibodies, chimeric antibodies, humanized antibodies, monospecific antibodies (such as bivalent monospecific antibodies), bispecific antibodies, antibodies of any isotype and/or allotype; antibody mixtures (recombinant polyclonals) for instance generated by technologies exploited by Symphogen and Merus (Oligoclonics), multimeric Fc proteins as described in WO2015/158867, and fusion proteins as described in WO2014/031646. While these different antibody fragments and formats are generally included within the meaning of antibody, they collectively and each independently are unique features of the present invention, exhibiting different biological properties and utility.

A “CD38 antibody” or “anti-CD38 antibody” as described herein is an antibody which binds specifically to the antigen CD38.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The terms “monoclonal antibody”, “monoclonal Ab”, “monoclonal antibody composition”, “mAb”, or the like, as used herein refer to a preparation of Ab molecules of single molecular composition. A monoclonal antibody composition displays a single binding specificity and affinity for a particular epitope. Accordingly, the term “human monoclonal antibody” refers to Abs displaying a single binding specificity which have variable and constant regions derived from human germline immunoglobulin sequences. The human mAbs may be generated by a hybridoma which includes a B cell obtained from a transgenic or trans-chromosomal non-human animal, such as a transgenic mouse, having a genome comprising a human heavy chain transgene repertoire and a light chain transgene repertoire, rearranged to produce a functional human antibody and fused to an immortalized cell.

As used herein, “isotype” refers to the immunoglobulin class that is encoded by heavy chain constant region genes, including, for instance, IgG1, IgG2, IgG3, IgG4, IgD, IgA1, IgA2, IgE, and IgM, as well as any allotypes thereof such as IgG1m(z), IgG1m(a), IgG1m(x), IgG1m(f) and mixed allotypes thereof such as IgG1m(za), IgG1m(zax), IgG1m(fa), etc. (see, for instance, de Lange, Experimental and Clinical Immunogenetics 1989; 6(1):7-17).

Further, each heavy chain isotype can be combined with either a kappa (κ) or lambda (I) light chain. The term “mixed isotype” used herein refers to Fc region of an immunoglobulin generated by combining structural features of one isotype with the analogous region from another isotype thereby generating a hybrid isotype. A mixed isotype may comprise an Fc region having a sequence comprised of two or more isotypes selected from the following IgG1, IgG2, IgG3, IgG4, IgD, IgA1, IgGA2, IgE, or IgM thereby generating combinations such as e.g. IgG1/IgG3, IgG1/IgG4, IgG2/IgG3, IgG2/IgG4 or IgG1/IgA.

The term “full-length antibody” when used herein, refers to an antibody (e.g., a parent or variant antibody) which contains all heavy and light chain constant and variable domains corresponding to those that are normally found in a wild-type antibody of the isotype in question.

A “full-length bivalent, monospecific monoclonal antibody” when used herein, refers to a bivalent, monospecific antibody (e.g., a parent or variant antibody) formed by a pair of identical HCs and a pair of identical LCs, with the constant and variable domains corresponding to those normally found in an antibody of the particular isotype in question.

The term “antigen-binding region”, “antigen binding region”, “binding region” or antigen binding domain, as used herein, refers to a region of an antibody which is capable of binding to the antigen. This binding region is typically defined by the VH and VL domains of the antibody which may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). The antigen can be any molecule, such as a polypeptide, e.g. present on a cell.

The term “target”, as used herein, refers to a molecule to which the antigen binding region of the antibody binds. The target includes any antigen towards which the raised antibody is directed. The term “antigen” and “target” may in relation to an antibody be used interchangeably and constitute the same meaning and purpose with respect to any aspect or embodiment of the present invention.

The term “epitope” means a protein determinant capable of specific binding to an antibody variable domain. Epitopes usually consist of surface groupings of molecules such as amino acids, sugar side chains or a combination thereof and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding (also called immunodominant component of the epitope) and other amino acid residues, which are not directly involved in the binding.

A “variant” as used herein refers to a protein or polypeptide sequence which differs in one or more amino acid residues from a parent or reference sequence. A variant may, for example, have a sequence identity of at least 80%, such as 90%, or 95%, or 97%, or 98%, or 99%, to a parent or reference sequence. Also or alternatively, a variant may differ from the parent or reference sequence by 12 or less, such as 11, 10, 9, 8, 7, 6, 5, 4, 3, 2 or 1 mutation(s) such as substitutions, insertions or deletions of amino acid residues. Accordingly, a “variant antibody” or an “antibody variant”, used interchangeably herein, refers to an antibody that differs in one or more amino acid residues as compared to a parent or reference antibody, e.g., in the antigen-binding region, Fc-region or both. Likewise, a “variant Fc region” or “Fc region variant” refers to an Fc region that differs in one or more amino acid residues as compared to a parent or reference Fc region, optionally differing from the parent or reference Fc region amino acid sequence by 12 or less, such as 11, 10, 9, 8, 7, 6, 5, 4, 3, 2 or 1 mutation(s) such as substitutions, insertions or deletions of amino acid residues. The parent or reference Fc region is typically the Fc region of a human wild-type antibody which, depending on the context, may be a particular isotype. A variant Fc region may, in dimerized form, be a homodimer or heterodimer, e.g., where one of the amino acid sequences of the dimerized Fc region comprises a mutation while the other is identical to a parent or reference wild-type amino acid sequence. Examples of wild-type (typically a parent or reference sequence) IgG CH and variant IgG constant region amino acid sequences, which comprise Fc region amino acid sequences, are set out in Table 4.

In the context of the present invention, conservative substitutions may be defined as substitutions within the following classes of amino acids:
Acidic Residues: Asp (D) and Glu (E)
Basic Residues: Lys (K), Arg (R), and His (H)
Hydrophilic Uncharged Residues: Ser (S), Thr (T), Asn (N), and Gln (Q)
Aliphatic Uncharged Residues: Gly (G), Ala (A), Val (V), Leu (L), and Ile (I)
Non-polar Uncharged Residues: Cys (C), Met (M), and Pro (P)
Aromatic Residues: Phe (F), Tyr (Y), and Trp (W)

Alternative conservative amino acid residue substitution classes:
1. A S T
2. D E
3. N Q
4. R K
5. I L M
6. F Y W

Alternative Physical and Functional Classifications of Amino Acid Residues:
Alcohol group-containing residues: S and T
Aliphatic residues: I, L, V, and M
Cycloalkenyl-associated residues: F, H, W, and Y
Hydrophobic residues: A, C, F, G, H, I, L, M, R, T, V, W, and Y
Negatively charged residues: D and E
Polar residues: C, D, E, H, K, N, Q, R, S, and T
Positively charged residues: H, K, and R
Small residues: A, C, D, G, N, P, S, T, and V
Very small residues: A, G, and S
Residues involved in turn formation: A, C, D, E, G, H, K, N, Q, R, S, P, and T
Flexible residues: Q, T, K, S, G, N, D, E, and R

“Sequence identity” as used herein refers to the percent identity between two sequences as a function of the number of identical positions shared by the sequences (i.e., percent homology=# of identical positions/total # of positions ×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) that has been incorporated into the ALIGN program (version 2.0), using a PAM 120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm. Other tools for sequence alignments are publicly available on the internet, and include, without limitation, Clustal Omega and EMBOSS Needle on the EMBL-EBI website www.ebi.ac.uk. Typically, default settings can be used.

In the context of the present invention the following notations are, unless otherwise indicated, used to describe a mutation; name of amino acid which is mutated, followed by the position number which is mutated, followed by what the mutation encompasses. Thus if the mutation is a substitution, the name of the amino acid which replaces the prior amino acid is included, if the amino acid is deleted it is indicated by a “*”, if the mutation is an addition the amino acid being added is included after the original amino acid. Amino acid names may be one or three-letter codes. Thus for example; the substitution of a glutamic acid in position 430 with a glycine is referred to as E430G, substitution of glutamic acid in position 430 with any amino acid is referred to as E430X, deletion of glutamic acid in position 430 is referred to as E430* and addition of a proline after glutamic acid at position E430 is referred to as E430EP.

As used herein, “immunosuppressive cells” refer to immune cells which may suppress an immune response in a subject, such as by suppressing the activity of effector T cells and/or inhibiting T cell proliferation. Examples of such immunosuppressive cells include, but are not limited to, regulatory T cells (Tregs), regulatory B cells (Bregs) and myeloid-derived suppressor cells (MDSCs). There are also immunosuppressive NK cells, NKT cells, macrophages and antigen-presenting cells (APCs). An example of a phenotype for an immunosuppressive NK cell is CD56^{bright}CD16⁻.

“Regulatory T cells” or “'Tregs” or “Treg” refers to T lymphocytes that regulate the activity of other T cell(s) and/or other immune cells, usually by suppressing their activity. An example of a Treg phenotype is CD3⁺CD4⁺CD25⁺CD127^{dim}. Tregs may further express Foxp3. It is appreciated that Tregs may not be fully restricted to this phenotype.

“Effector T cells” or “Teffs” or “Teff” refers to T lymphocytes that carry out a function of an immune response, such as killing tumor cells and/or activating an antitumor immune-response which can result in clearance of the tumor cells from the body. Examples of Teff phenotypes include CD3⁺CD4⁺ and CD3⁺CD8⁺. Teffs may secrete, contain or express markers such as IFNγ, granzyme B and ICOS. It is appreciated that Teffs may not be fully restricted to these phenotypes.

“Myeloid-derived suppressor cells” or “MDSCs” or “MDSC” refers to a specific population of cells of the hematopoietic lineage that express the macrophage/monocyte marker CD11 b and the granulocyte marker Gr-1/Ly-6G. An example of an MDSC phenotype is CD11b⁺HLA-DR⁻CD14⁻CD33⁺CD15⁺. MDSCs typically also show low or undetectable expression of the mature antigen presenting cell markers MHC Class II and F480. MDSCs are immature cells of the myeloid lineage and may further differentiate into other cell types, such as macrophages, neutrophils, dendritic cells, monocytes or granulocytes. MDSCs may be found naturally in normal adult bone marrow of human and animals or in sites of normal hematopoiesis, such as the spleen.

“Regulatory B cell” or “Breg” or “Bregs” refers to B lymphocytes that suppress immune responses. An example of a Breg phenotype is CD19⁺CD24⁺CD38⁺. Bregs may suppress immune responses by inhibiting T cell proliferation mediated by IL-10 secreted by the Bregs. It is appreciated that other Breg subsets exists, and are described in for example Ding et al., (2015) Human Immunology 76: 615-621.

As used herein, the term “effector cell” refers to an immune cell which is involved in the effector phase of an immune response. Exemplary immune cells include a cell of a myeloid or lymphoid origin, for instance lymphocytes (such as B cells and T cells including cytolytic T cells (CTLs)), killer cells, natural killer cells, macrophages, monocytes, eosinophils, polymorphonuclear cells, such as neutrophils, granulocytes, mast cells, and basophils. Some effector cells express Fc receptors (FcRs) or complement receptors and carry out specific immune functions. In some embodiments, an effector cell such as, e.g., a natural killer cell, is capable of inducing ADCC. For example, monocytes, macrophages, neutrophils, dendritic cells and Kupffer cells which express FcRs, are involved in specific killing of target cells and/or presenting antigens to other components of the immune system, or binding to cells that present antigens. In some embodiments the ADCC can be further enhanced by antibody driven classical complement activation resulting in the deposition of activated C3 fragments on the target cell. C3 cleavage products are ligands for complement receptors (CRs), such as CR3, expressed on myeloid cells. The recognition of complement fragments by CRs on effector cells may promote enhanced Fc receptor-mediated ADCC. In some embodiments antibody driven classical complement activation leads to C3 fragments on the target cell. These C3 cleavage products may promote direct complement-dependent cellular cytotoxicity (CDCC). In some embodiments, an effector cell may phagocytose a target antigen, target particle or target cell which may depend on antibody binding and mediated by FcγRs expressed by the effector cells. The expression of a particular FcR or complement receptor on an effector cell may be regulated by humoral factors such as cytokines. For example, expression of FcγRI has been found to be up-regulated by interferon γ (IFNγ) and/or G-CSF. This enhanced expression increases the cytotoxic activity of FcγRI-bearing cells against targets. An effector cell can phagocytose a target antigen or phagocytose or lyse a target cell. In some embodiments antibody driven classical complement activation leads to C3 fragments on the target cell. These C3 cleavage products may promote direct phagocytosis by effector cells or indirectly by enhancing antibody mediated phagocytosis.

The term “Fc effector functions,” as used herein, is intended to refer to functions that are a consequence of binding a polypeptide or antibody to its target, such as an antigen, on a cell membrane wherein the Fc effector function is attributable to the Fc region of the polypeptide or antibody. Examples of Fc effector functions include (i) C1q-binding, (ii) complement activation, (iii) complement-dependent cytotoxicity (CDC), (iv) antibody-dependent cell-mediated cytotoxity (ADCC), (v) Fc-gamma receptor-binding, (vi) antibody-dependent cellular phagocytosis (ADCP), (vii) complement-dependent cellular cytotoxicity (CDCC), (viii) complement-enhanced cytotoxicity, (ix) binding to complement receptor of an opsonized antibody mediated by the antibody, (x) opsonisation, (xi) trogocytosis, and (xii) a combination of any of (i) to (xi).

As used herein, the term “complement activation” or “activation of the complement system” refers to the activation of the classical complement pathway, which is initiated by a large macromolecular complex called C1 binding to antibody-antigen complexes on a surface. C1 is a complex, which consists of 6 recognition proteins C1q and a hetero-tetramer of serine proteases, C1 r2C1s2. C1 is the first protein complex in the early events of the classical complement cascade that involves a series of cleavage reactions that starts with the cleavage of C4 into C4a and C4b and C2 into C2a and C2b. C4b is deposited and forms together with C2a an enzymatic active convertase called C3 convertase, which cleaves complement component C3 into C3b and C3a, which forms a C5 convertase This C5 convertase splits C5 in C5a and C5b and the last component is deposited on the membrane and that in turn triggers the late events of complement activation in which terminal complement components C5b, C6, C7, C8 and C9 assemble into the membrane attack complex (MAC). The complement cascade results in the creation of pores in the cell membrane which causes lysis of the cell, also known as complement-dependent cytotoxicity (CDC). Complement activation can be evaluated by using C1q efficacy, CDC kinetics CDC assays (as described in WO2013/004842, WO2014/108198) or by the method Cellular deposition of C3b and C4b described in Beurskens et al., J Immunol Apr. 1, 2012 vol. 188 no. 7, 3532-3541.

Complement activation in a subject can be measured by determining the level of e.g. C2 or CH50 according to any method known in the art, e.g. C2 levels may be determined in plasma using Radial Immunodiffusion (RID) assay, and CH50 (complement lytic activity) may be measured in serum using a spectrophotometric assay using the Autokit CH50.

The term “complement-dependent cytotoxicity” (CDC), as used herein, is intended to refer to the process of antibody-mediated complement activation leading to lysis of the cell to which the antibody is bound, which, without being bound by theory is believed to be the result of pores in the membrane that are created by the assembly of the so-called membrane attack complex (MAC). Suitable assays for evaluating CDC are known in the art and include, for example, in vitro assays in which normal human serum is used as a complement source, as described in Example 3. A non-limiting example of an assay for determining the maximum lysis of CD38 expressing cells as mediated by a CD38 antibody, or the EC50 value, may comprise the steps of:
(a) plating about 100,000 CD38-expressing cells in 40 μL culture medium supplemented with 0.2% BSA per well in a multi-well plate;
(b) preincubating cells for 20 minutes with 40 μL of serially diluted CD38 antibody (0.0002-10 μg/mL);
(c) incubating each well for 45 minutes at 37° C. with 20 percent of pooled normal human serum;
(d) adding a viability dye and measuring the percentage of cell lysis on a flow cytometer;
(e) determining the maximum lysis and/or calculating the EC50 value using non-linear regression.

The term “antibody-dependent cell-mediated cytotoxicity” (“ADOC”) as used herein, is intended to refer to a mechanism of killing of antibody-coated target cells by cells expressing Fc receptors that recognize the constant region of the bound antibody. Suitable assays for evaluating ADCC are known in the art and include, for example, the assays described in Example 4. Non-limiting examples of assays for determining the ADCC of CD38-expressing cells as mediated by a CD38 antibody may comprise the steps of the ⁵¹Cr-release assay or the reporter assay set out below.

ADCC with ⁵¹Cr Release Assay
(a) plating about 5,000 ⁵¹Cr labelled CD38-expressing cells (e.g., Daudi cells) in 50 μL culture medium supplemented with 0.2% BSA per well in a multi-well plate;
(b) preincubating cells for 15 minutes with 50 μL of serially diluted CD38 antibody (0.0002-10 μg/mL);
(c) incubating each well for 4 hours at 37° C. with 500,000 freshly isolated peripheral blood mononuclear cells (PBMCs) per well;
(d) measuring the amount of ⁵¹Cr release in 75 μL supernatant on a gamma counter;
(e) calculating the percentage of cell lysis as (cpm sample −cpm spontaneous lysis)/(cpm maximal lysis −cpm spontaneous lysis) wherein cpm is counts per minute.
   ADCC with Reporter Assay
(a) plating about 5,000 CD38-expressing cells (e.g., Daudi cells) in 10 μL in multi-well plates suitable for optical readings (e.g., 384-well OptiPlates from PerkinElmer Inc.) in a standard medium (e.g., RPMI 1640) supplemented with 25% low IgG serum;
(b) incubating each well for 6 hours at 37° C. with 10 μL engineered Jurkat cells stably expressing the FcγRIIIa receptor, V158 (high affinity) variant, and an NFAT response element driving expression of firefly luciferase as effector cells and 10 μL serially diluted CD38 antibody (0.0002-10 μg/mL);
(c) incubating each well 5 minutes at RT with 30 μL Luciferase substrate and measuring luminescence.

The term “antibody-dependent cellular phagocytosis” (“ADCP”) as used herein is intended to refer to a mechanism of elimination of antibody-coated target cells by internalization by phagocytes. The internalized antibody-coated target cells are contained in a vesicle called a phagosome, which then fuses with one or more lysosomes to form a phagolysosome. Suitable assays for evaluating ADCP are known in the art and include, for example, the in vitro cytotoxicity assay with macrophages as effector cells and video microscopy as described by van Bij et al. in Journal of Hepatology Volume 53, Issue 4, October 2010, Pages 677-685, and the in vitro cytotoxicity assay described in Example 5. A non-limiting example of an assay for determining the ADCP of CD38 expressing cells as mediated by a CD38 antibody may comprise the steps of:
(a) differentiating freshly isolated monocytes to macrophages with 5 days incubation in GM-CSF-containing medium;
(b) plating about 100,000 macrophages per well in a multi-well plate in dendritic cell medium with GM-CSF;
(c) adding 20,000 CD38-antibody opsonized CD38-expressing cells (e.g., Daudi cells), labelled with a generic fluorescent membrane dye, per well for 45 minutes at 37° C.;
(d) measuring the percentage of CD14-positive, CD19-negative, membrane-dye-positive macrophages on a flow cytometer.

As used herein, “trogocytosis” refers to a process characterized by the transfer of cell surface molecules from a donor cell to an acceptor cell, such as an effector cell. Typical acceptor cells include T and B cells, monocytes/macrophages, dendritic cells, neutrophils, and NK cells. Trogocytosis-mediated transfer of a cell surface molecule such as, e.g., CD38, from a donor cell to an acceptor cell may also result in the transfer of an antibody-antigen complex from the donor cell to an acceptor cell, i.e., an antibody-antigen complex where an antibody is bound to the cell surface molecule. In particular, a specialized form of trogocytosis may occur when the acceptor cells are Fc-gamma-receptor (FcγR) expressing effector cells; these acceptor cells may take up and internalize donor cell-associated immune complexes composed of specific antibodies bound to target antigens on donor cells, typically after binding of FcγRs to the Fc regions of the antibodies. Suitable assays for evaluating trogocytosis are known in the art and include, for example, the assay in Example 8. Non-limiting examples of assays for determining trogocytosis of CD38 expressing cells as mediated by a CD38 antibody include the following:

### Trogocytosis (Daudi Cells):

(a′) differentiating freshly isolated monocytes to macrophage with 5 days GM-CSF;
(b′) plating about 100,000 macrophages per well in dendritic cell medium with GM-CSF;
(c′) adding about 20,000 CD38 antibody-opsonized Daudi cells, labelled with a generic fluorescent membrane dye, per well for 45 minutes at 37° C.;
(d′) measuring CD38 expression on Daudi cells on a flow cytometer, wherein a reduction in CD38 on CD38-antibody opsonized Daudi cells as compared to a control indicates trogocytosis.

### Trogocytosis (Tregs):

(a) plating about 500,000 freshly isolated PBMCs per well in cell culture medium O/N at 37° C.;
(b) adding about 100,000, CD38 antibody-opsonized Tregs, labelled with a generic fluorescent intracellular amine dye, per well overnight (0/N) at 37° C.; and
(c) measuring CD38 expression on Tregs on a flow cytometer, wherein a reduction in CD38 on CD438-antibody opsonized Tregs as compared to a control indicates trogocytosis.

The control can be selected by the skilled person based on the specific purpose of the study or assay in question. However, non-limiting examples of controls include (i) the absence of any antibody and (ii) an isotype control antibody. One example of an isotype control antibody is antibody b12, having the VH and VL sequences described in Table 4. In some embodiments where it is desired to evaluate the trogocytosis-effect of an antibody as described herein, the control may be (iii) a parent or reference antibody having a different antigen-binding region and/or a different Fc region.

In some embodiments, in step (b), in addition or alternative to the fluorescent intracellular amine dye, the Tregs are labelled with a generic fluorescent membrane dye.

In some embodiments, in step (d′) and (c) of the trogocytosis assays outlined above, the reduction in CD38 antibody on the donor cells can also be measured. For example, in cases where the CD38 antibody is a human IgG (huIgG) antibody, a secondary antibody can be used to detect huIgG.

In addition to Daudi cells (ATCC CCL-213), tumor cells suitable for the first assay include, without limitation, those listed in Table 2 of WO 2020/012036 A1 (incorporated herein by reference), particularly those with a high CD38 expression.

In addition to Tregs, suitable CD38-expressing cells for the second assay include immune cells such as, e.g., NK cells, B cells, T cells and monocytes, as well as tumor cells listed in Table 2 of WO 2020/012036 A1, particularly those with a low CD38 expression level.

The term “vector,” as used herein, is intended to refer to a nucleic acid molecule capable of inducing transcription of a nucleic acid segment ligated into the vector. One type of vector is a “plasmid”, which is in the form of a circular double stranded DNA loop. Another type of vector is a viral vector, wherein the nucleic acid segment may be ligated into the viral genome. Certain vectors are capable of autonomous replication in a host cell into which they are introduced (for instance bacterial vectors having a bacterial origin of replication and episomal mammalian vectors). Other vectors (such as non-episomal mammalian vectors) may be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present invention is intended to include such other forms of expression vectors, such as viral vectors (such as replication defective retroviruses, adenoviruses and adeno-associated viruses), which serve equivalent functions.

The term “recombinant host cell” (or simply “host cell”), as used herein, is intended to refer to a cell into which one or more expression vectors have been introduced. For example, the HC and LC of an antibody as described herein may both be encoded by the same expressing vector, and a host cell transfected with the expression vector. Alternatively, the HC and LC of an antibody as described herein may be encoded by different expression vectors, and a host cell co-transfected with the expression vectors. It should be understood that the term “host cell” is intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, transfectomas, such as CHO cells, HEK-293 cells, PER.C6, NS0 cells, and lymphocytic cells, and prokaryotic cells such as E. coli and other eukaryotic hosts such as plant cells and fungi.

The term “transfectoma”, as used herein, includes recombinant eukaryotic host cells expressing the Ab or a target antigen, such as CHO cells, PER.C6, NS0 cells, HEK-293 cells, plant cells, or fungi, including yeast cells.

The term “treatment” refers to the administration of an effective amount of a therapeutically active antibody of the present invention with the purpose of easing, ameliorating, arresting or eradicating (curing) symptoms or disease states.

The term “effective amount” or “therapeutically effective amount” refers to an amount effective, at dosages and for periods of time necessary, to achieve a desired therapeutic result. A therapeutically effective amount of an antibody may vary according to factors such as the disease state, age, sex, and weight of the individual, and the ability of the antibody to elicit a desired response in the individual. A therapeutically effective amount is also one in which any toxic or detrimental effects of the antibody are outweighed by the therapeutically beneficial effects.

As used herein, the terms “binding” or “capable of binding” in the context of the binding of an antibody to a predetermined antigen or epitope typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, when determined using Bio-Layer Interferometry (BLI) or, for instance, when determined using surface plasmon resonance (SPR) technology in a BIAcore 3000 instrument using the antigen as the ligand and the antibody as the analyte. The antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., BSA, casein) other than the predetermined antigen or a closely-related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “k_{d}” (sec¹), as used herein, refers to the dissociation rate constant of a particular antibody-antigen interaction. Said value is also referred to as the k_{off} value.

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

“Treatment cycle” is herein defined as the time period, within the effects of separate dosages of the antibody adds on due to its pharmacodynamics, or in other words the time period after which the subject's body is essentially cleared from or being cleared from the administrated antibody. Multiple small doses in a small time window; e.g. within 2-24 hours, such as 2-12 hours or on the same day, might be equal to a larger single dose.

Efficacy assessment for MM of the treatment may be determined according to the 2016 International Myeloma Working Group (IMWG) Uniform Response Criteria for Response and Minimal Residual Disease Assessment in Multiple Myeloma (Kumar et al., 2016), as show in table 1 below.

TABLE 1
2016 International Myeloma Working Group (IMWG) Uniform Response Criteria for Response
and Minimal Residual Disease Assessment in Multiple Myeloma (Kumar et al., 2016)

| | |
|---|---|
| Response | |
| Subcategory | Response Criteria |

IMWG criteria for response assessment including criteria for MRD¹
IMWG MRD criteria (requires a complete response as defined below)

| | |
|---|---|
| Sustained MRD- | MRD negativity in the marrow (NGF, NGS, or both) and by imaging as defined below, confirmed |
| negative | minimum of 1 year apart. Subsequent evaluations can be used to further specify the duration of |
| | negativity (eg, MRD-negative at 5 years)² |
| Flow MRD- | Absence of phenotypically aberrant clonal plasma cells by NGF³ on bone marrow aspirates using |
| negative | the EuroFlow standard operation procedure for MRD detection in MM (or validated equivalent |
| | method) with a minimum sensitivity of 1 in 10⁵ nucleated cells or higher |
| Sequencing | Absence of clonal plasma cells by NGS or bone marrow aspirate in which presence of a clone is |
| MRD-negative | defined as less than 2 identical sequencing reads obtained after DNA sequencing of bone marrow |
| | aspirates using the LymphoSIGHT platform (or validated equivalent method) with a minimum |
| | sensitivity of 1 in 10⁵ nucleated cells⁴ or higher |
| Imaging plus | MRD negativity as defined by NGF or NGS plus disappearance of every area of increased tracer |
| MRD-negative | uptake found at baseline or a preceding PET/CT or decrease to less mediastinal blood pool SUV |
| | or decrease to less than that of surrounding normal tissue⁵ |

Standard IMWG response criteria^{1, 6}

| | |
|---|---|
| sCR | Complete response as defined below plus normal FLC ratio⁷ and absence of clonal cells in bone |
| | marrow biopsy by IHC (κ/λ ratio ≤4:1 or ≥1:2 for κ and λ patients, respectively, after counting ≥100 |
| | plasma cells^{8, 9} |
| CR | Negative IFE on the serum and urine and disappearance of any soft tissue plasmacytomas |
| | and ≤5% plasma cells in bone marrow aspirates |
| VGPR | Serum and urine M-protein detectable by IFE but not on electrophoresis or ≥90% reduction in |
| | serum M-protein plus urine M-protein level <100 mg per 24 h |
| PR | ≥50% reduction of serum M-protein plus reduction in 24-h urinary M-protein by ≥90% or to <200 mg |
| | per 24 h; |
| | If the serum and urine M-protein are unmeasurable, a ≥50% decrease in the difference |
| | between involved and uninvolved FLC levels is required in place of the M-protein criteria; |
| | If serum and urine M-protein are unmeasurable, and serum-free light assay is also |
| | unmeasurable, ≥50% reduction in plasma cells is required in place of M-protein, provided |
| | baseline bone marrow plasma-cell percentage was ≥30%. |
| | In addition to these criteria, if present at baseline, a ≥50% reduction in the size (SPD)¹⁰ of soft |
| | tissue plasmacytomas is also required |
| MR | ≥25% but ≤49% reduction of serum M-protein and reduction of 24-h urine by 50% to 89%. In |
| | addition to the above listed criteria, if present at baseline, a ≥>50% reduction in the size (SPD)¹⁰ of |
| | soft tissue plasmacytomas is also required. |
| SD | Not recommended for use as an indicator of response; stability of disease is best described by |
| | providing the time-to-progression estimates. Not meeting criteria for CR, VGPR, PR, MR, or PD |
| PD^{11, 12} | Any 1 or more of the following criteria: |
| | Increase of ≥25% from lowest confirmed response value in 1 or more of the following criteria: |
| | Serum M-protein (absolute increase must be ≥0.5 g/dL); |
| | Serum M-protein increase ≥1 g/dL, if the lowest M component was ≥5 g/dL; |
| | Urine M-protein (absolute increase must be ≥200 mg/24 hr). |
| | In patients without measurable serum and urine M-protein levels, the difference between |
| | involved and uninvolved FLC levels (absolute increase must be >10 mg/dL); |
| | In patients without measurable serum and urine M-protein levels and without measurable |
| | involved FLC levels, bone marrow plasma-cell percentage irrespective of baseline status |
| | (absolute increase must be ≥10%); |
| | Appearance of a new lesion(s), ≥50% increase from nadir in SPD of >1 lesion, or ≥50% |
| | increase in the longest diameter of the previous lesion >1 cm in short axis; |
| | ≥50% increase in circulating plasma cells (minimum of 200 cells per μL) if this is the only |
| | measure of disease |
| Clinical Relapse | Clinical relapse requires 1 or more of the following: |
| | Direct indicators of increasing disease and/or end organ dysfunction (CRAB features) related |
| | to the underlying clonal plasma-cell proliferative disorder. It is not used in calculation of time |
| | to progression or PFS but is listed here as something that can be reported optionally or for use |
| | in clinical practice; |
| | Development of new soft tissue plasmacytomas or bone lesions (osteoporotic fractures do not |
| | constitute progression); |
| | Definite increase in the size of existing plasmacytomas or bone lesions. A definite increase is |
| | defined as a 50% (and ≥1 cm) increase as measured serially by the SPD¹⁰ of the measurable |
| | lesion; |
| | Hypercalcemia (>11 mg/dL) |
| | Decrease in hemoglobin of ≥2 g/dL, not related to therapy or other non-myeloma related |
| | conditions; |
| | Rise in serum creatinine by 2 mg/dL or more from the start of therapy and attributable to |
| | myeloma; |
| | Hyperviscosity related to serum paraprotein |
| Relapse from CR | Any 1 or more of the following: |
| (To be used only if | Reappearance of serum or urine M-protein by IFE or electrophoresis; |
| the end point | Development of ≥5% plasma cells in the bone marrow; |
| studied is | Appearance of any other sign of progression (ie, new plasmacytoma, lytic bone lesion, or |
| disease-free | hypercalcemia) |
| survival) | |
| Relapse from | Any 1 or more of the following: |
| MRD negative | Loss of MRD-negative state (evidence of clonal plasma cells on NGF or NGS, or positive |
| (To be used only if | imaging study for recurrence of myeloma; |
| the end point is | Reappearance of serum or urine M-protein by IFE or electrophoresis; |
| disease-free | Development of ≥5% plasma cells in the bone marrow; |
| survival) | Appearance of any other sign of progression (ie, new plasmacytoma, lytic bone lesion, or |
| | hypercalcemia) |

CR = complete response;
CRAB = calcium elevation, renal failure, anaemia, lytic bone lesions;
DFS = disease-free survival;
CT = computed tomography;
FCM = flow cytometry;
FDG = fluorodeoxyglucose;
FLC = free light chain;
h = hour(s);
IFE = immunofixation;
IHC = immunohistochemistry;
mAb = monoclonal antibody;
MM = multiple myeloma;
MR = minimal response;
MRD = minimal residual disease;
NGF = next generation flow;
NGS = next generation sequencing;
MFC = multiparameter flow cytometry;
PD = progressive disease;
PET = positron emission tomography;
PFS = progression-free survival;
PR = partial response;
sCR = stringent complete response;
SD = stable disease;
SPD = sum of the product of the maximal perpendicular diameters of measured lesions;
SUV = standardized uptake value;
SUVmax = maximum standardized uptake value;
VGPR = very good partial response

1. All response categories require 2 consecutive assessments made at any time before the institution of any new therapy; for MRD there is no need for 2 consecutive assessments, but information on MRD after each treatment stage is recommended (eg, after induction, high-dose therapy/ASCT, consolidation, maintenance); MRD tests should be initiated only at the time of suspected CR. All categories of response and MRD require no known evidence of progressive or new bone lesions if radiographic studies were performed. However, radiographic studies are not required to satisfy these requirements of FDG PET if imaging MRD-negative status is reported.
2. Sustained MRD negativity when reported should also annotate the method used (eg, sustained flow MRD-negative, sustained sequencing MRD-negative).
3. Bone marrow MFC should follow NGF guidelines. The reference NGF method is an 8-color 2 tube approach, which has been extensively validated. The 2-tube approach improves reliability, consistency, and sensitivity because of the acquisition of a greater number of cells. The 8-color technology is widely available globally and the NGF method has already been adopted in many flow laboratories worldwide. The complete 8-color method is most efficient using a lyophilized mixture of antibodies which reduces errors, time, and costs. 5 million cells should be assessed. The FCM method employed should have a sensitivity of detection of at least 1 in 105 plasma cells.
4. DNA sequencing assay on bone marrow aspirate should use a validated assay such as LymphoSIGHT (Sequenta).
5. Criteria used by Zamagni and colleagues, and expert panel (IMPetUs; Italian Myeloma Criteria for PET use). Baseline positive lesions were identified by presence of focal areas of increased uptake within bones, with or without any underlying lesion identified by CT and present on at least 2 consecutive slices. Alternatively, an SUVmax=2.5 within osteolytic CT areas >1 cm in size, or SUVmax=1.5 within osteolytic CT areas 1≤cm in size were considered positive. Imaging should be performed once MRD negativity is determined by MFC or NGS.
6. Derived from international uniform response criteria. Minor response definition and clarifications derived from Rajkumar and colleagues. When the only method to measure disease is by serum FLC levels: CR can be defined as a normal FLC ration 0-26 to 1-65 in additional to the CR criteria listed previously. VGPR in such patients requires ≥90% decrease in the difference between involved and uninvolved FLC levels. All response categories require 2 consecutive assessments made at any time before the institution of any new therapy; all categories also require no known evidence of progressive or new bone lesions or extramedullary plasmacytomas if radiographic studies were performed. Radiographic studies are not required to satisfy these response requirements. Bone marrow assessments do not need to be confirmed. Each category, except for SD, will be considered unconfirmed until the confirmatory test is performed. The date of initial test is considered as the date of response for evaluation of time dependent outcomes such as duration of response.
7. All recommendations regarding clinical uses relating to serum FLC levels and FLC ratio are based on results obtained with validated Freelight test (Binding Site, Birmingham, UK).
8. Presence/absence of clonal cells on IHC is based upon the κ/λ/L ratio. An abnormal κ/λ ratio by IHC requires a minimum of 100 plasma cells for analysis. An abnormal ratio reflecting presence of an abnormal clone is κ/λ of >4:1 or <1:2.
9. Special attention should be given to the emergence of a different monoclonal protein following treatment, especially in the setting of patients having achieved a conventional CR, often related to oligoclonal reconstitution of the immune system. These bands typically disappear over time and in some studies have been associated with a better outcome. Also, appearance of monoclonal IgG κ in patients receiving mAbs should be differentiated from the therapeutic antibody.
10. Plasmacytoma measurements should be taken from the CT portion of the PET/CT, or MRI scans, or dedicated CT scans where applicable. For patients with only skin involvement, skin lesions should be measured with a ruler. Measurement of tumor size will be determined by SPD.
11. Positive IFE alone in a patient previously classified as achieving a CR will not be considered progression. For purposes of calculating time to progression and PFS, patients who have achieved a CR and are MRD-negative should be evaluated using criteria listed for PD. Criteria for relapse from a CR or relapse from MRD should be used only when calculating disease-free survival.
12. In the case where a value is felt to be a spurious result per physician discretion (eg, a possible laboratory error), that value will not be considered when determining the lowest value.

The “best overall response” (BOR), as used herein, is the best response recorded during the course of treatment while on trial. Subjects with sCR, CR, VGPR or PR are considered to have objective response.

“Objective response rate (ORR)”, as used herein, refers to the proportion of subjects with a partial response, or better (e.g. PR, VGPR, CR or sCR).

“Clinical benefit rate”, as used herein, (CBR) is defined as the proportion of subjects with at least stable disease.

“Duration of response (DOR)”, as used herein, only applies to subjects whose confirmed best overall response is PR or better (e.g. PR, VGPR, CR or sCR) and is defined as the time from the first documentation of objective tumor response (e.g. PR, VGPR, CR or sCR) to the date of MR, SD or PDor death due to underlying cancer.

“Time to Response (TTR)”, as used herein, is defined as time from C1 D1 to occurrence of a response (PR or better). TTR will be summarized and presented descriptively for those with a response (PR or better).

“Progression-free survival (PFS)” is defined as the time, e.g. number of days, from Day 1 in Cycle 1 (C1D1) to the first documented progression or death due to any cause.

“Overall survival (OS)”, as used herein, is defined as the time, e.g. number of days, from Day 1 in Cycle 1 (C1D1) to death due to any cause. If a subject is not known to have died, then OS will be censored at the latest date the subject was known to be alive (on or before the cut-off date).

“Adverse Events (AE)”, as used herein, is any untoward medical occurrence in a patient or clinical trial subject, temporally associated with the use of a medicinal product, whether or not considered related to the medicinal product. An AE can therefore be any unfavorable and unintended sign (including an abnormal laboratory finding), symptom, or disease (new or exacerbated) temporally associated with the use of a medicinal product. The severity of AEs are described according to the National Cancer Institute's Common Terminology Criteria for Adverse Events (CTCAE), v5.0 (Common Terminology Criteria for Adverse Events (CTCAE), v5.0. Available at: https://ctep.cancer.gov/protocolDevelopment/electronic_applications/docs/CTCAE_v5_Quick_Reference_8.5×11.pdf.), except for TLS (tumor lysis syndrome), which are graded according to Cairo-Bishop et al (Coiffier, B., Altman, A., Pui, C. H., Younes, A., and Cairo, M. S. (2008). Guidelines for the management of pediatric and adult tumor lysis syndrome: an evidence-based review. J Clin Oncol 26, 2767-2778). AEs will include only AEs that started or pre-existing AEs that worsened during the on-treatment period, ie, TEAEs.

AEs, as used herein, include “serious adverse events (SAEs)”, which are defined as an AE that meets at least one of the following criteria:
Is fatal or life-threatening
Results in persistent or significant disability/incapacity
Constitutes a congenital anomaly/birth defect
Is medically significant, ie, defined as an event that jeopardizes the subject or may require medical or surgical intervention to prevent one of the outcomes listed above
Requires inpatient hospitalization or prolongation of existing hospitalization

In the definition of SAEs, the term “life-threatening” refers to an event in which the subject was at risk of death at the time of the event; it does not refer to an event which hypothetically might have caused death if it were more severe.

Further in the definition of SAEs, hospitalizations for the following reasons should not be reported as SAEs:
Routine treatment or monitoring of the underlying disease
Solely due to progression of the underlying cancer
Elective or pre-planned treatment for a pre-existing condition that is unrelated to the underlying disease and has not worsened since signing the informed consent
Social reasons and respite care in the absence of any deterioration in the subject's general condition
Treatment on an emergency outpatient basis that does not result in hospital admission and involves an event not fulfilling any of the definitions of a SAE given above is not an SAE.

AEs also included are “Adverse events of special interest (AESI)”, which are defined as events (serious or non-serious) which are of scientific and medical concern specific to the sponsor's product or program, for which ongoing monitoring and rapid communication by the investigator to the sponsor may be appropriate. Such events may require further investigation in order to characterize and understand them. Infusion related reactions (IRRs) will be considered AESIs. Other AESIs are defined on the basis of an ongoing review of the safety data.

“Infusion related reactions” (IRRs), as used herein, are defined as any AEs occurring during infusion or where the onset of the event occurs within 24 hours after the end of the infusion. For IRRs, the causality of the event should be judged as “related” by the investigator.

“Tumor lysis syndrome” (TLS), as used herein, is characterized by a group of metabolic derangements caused by the massive and abrupt release of cellular components into the blood after the rapid lysis of malignant cells, and is defined according to the Cairo-Bishop classification (Coiffier et al., 2008, supra).

“Dose limiting toxicity” (DLT), as used herein, refers to any AE of specified grades except those that are clearly and incontrovertibly due to the underlying disease or extraneous causes. In relation hereto, the DLT Evaluation Period is defined as the first 28 days of treatment (ie, Cycle 1). Toxicities will be graded for severity according to the National Cancer Institute Common Terminology Criteria for Adverse Events (NCI-CTCAE), v5.0, with the exception of TLS. A subject is said to be “DLT evaluable” after meeting minimum exposure criterion (i.e., receives 4 out of 4 preplanned doses during the DLT Evaluation Period. Note, the initial 2 split doses in Day 1 and Day 2 account for 1 preplanned dose.) and have sufficient safety evaluations (i.e. completing the DLT Evaluation Period), or experience a DLT during the first 28 days of dosing (i.e., in Cycle 1). Subjects not meeting the description for “DLT evaluable” are considered as “non-DLT evaluable”.

“Maximum tolerated dose” (MTD), as used herein, refers to the highest dose level (DL) that produces an acceptable level of toxicity (ie, within the target toxicity range) based on a mBOIN algorithm.

As used herein “not previously treated with”, refers to a subject that has not received a prior anti-cancer therapy (for the particular hematological malignancy), i.e. that is naïve for a certain therapy. For example, a subject that has not previously been treated with an (any) anti-CD38 antibody, is anti-CD38-naïve, a subject that has not previously been treated with daratumumab, is daratumumab-naïve (“dara-naïve”), and a subject that has not previously been treated with isatuximab, is isatuximab-naïve (“isa-naïve”). A subject that is anti-CD38-naïve may or may not have had a prior anti-cancer therapy with an agent different from an anti-CD38 antibody, but has not received a prior therapy with an anti-CD38 antibody. A subject that is dara-naïve, may or may not have had a prior anti-cancer therapy, but has not had a prior therapy with an anti CD38 antibody. Likewise, a subject that is isa-naïve may or may not have had a prior anti-cancer therapy, but has not had a prior therapy with an anti CD38 antibody.

In the context of the present invention, the term “treatment regimen” refers to a structured treatment plan designed to improve and maintain health.

The terms “pharmaceutical composition” and “pharmaceutical formulation” is used interchangeably herein.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means a range of up to 5%.

### Specific Embodiments of the Invention

The various embodiments of the invention are described herein after. The present invention is based on the observation that anti-CD38 antibodies comprising a mutation in one or more amino acid residues selected from the group corresponding to E430, E345 and S440 in a human IgG1 heavy chain, such as E430G, in particular antibody IgG1-C-E430G, are safe and well tolerable when used in humans, in particular in the treatment of hematological malignancies such as multiple myeloma (MM), and show biological activity and efficacy as of a dose level of 4 mg/kg body weight.

As established during the dose escalation, no dose level toxicity (DLT) was observed at dose levels ranging from 0.2/0.6 mg/kg up to 16 mg/kg. No tumor lysis syndrome (TLS) or cytokine release syndrome (CRS) was observed up to a dose level of 24 mg/kg. The most common treatment-emergent adverse events (AEs) were infusion-related reactions (IRRs; 75.0%), neutropenia (62.5%), anemia (41.7%), diarrhea (41.7%), COVID-19 (25.0%), pyrexia (25.0%), thrombocytopenia (20.8%), and vision blurred (20.8%). IRRs were mostly low grade (G; G1-2: 58.3%, G3: 16.7%, no G4), mainly occurred during the first infusion, and were manageable.

Among 19 response-evaluable patients in the dose escalation (n=5 naive to anti-CD38 mAb; n=16 refractory to anti-CD38 mAb), preliminary antitumor activity was seen. In patients naive to anti-CD38 mAb, 2 patients achieved complete response (CR; 1 each at 4 mg/kg and 24 mg/kg); 1 achieved minimal response (MR; 16 mg/kg). In patients refractory to anti-CD38 mAb, 1 patients achieved PR (16 mg/kg) and 2 achieved MR (1 each at 8 mg/kg and 16 mg/kg).

Biological activity of the antibody was confirmed at all evaluated dose levels during the dose escalation. A rapid, sustained decrease in peripheral blood natural killer cells was observed at all doses in all patients (median 97%; range 66-100% peak reduction from baseline [BL], n=21, pts with only a BL assessment were excluded from this analysis). T cells transiently decreased after administration of first doses 4 mg/kg, followed by expansion (≥100% increase from BL), particularly in patients naive to anti-CD38 mAb. A transient reduction in complement component C2 (median 64%; range 6-78% peak reduction from BL, n=18, pts with BL value below LLOQ, no BL value or only a BL value were excluded from this analysis) and total complement lytic activity (median 53%; range 2-92% peak reduction from BL for ≥8 mg/kg, n=11, pts with BL value below LLOQ, no BL value or only a BL value were excluded from this analysis) was induced at all evaluable doses, suggesting CDC. Complement parameters rapidly returned to BL, indicating that treatment does not exhaust complement. Plasma cytokine (IL-2, IL-6, IL-8, IL-10, IFNγ, and TNFα) levels generally remained low after treatment. Notably, complement activation appeared enhanced compared to a prior CD38 antibody at similar dose levels (Nijhof et al., Blood 2016 Aug. 18; 128(7):959-70).

Remarkably, no dose dependent reduction in CD38-expressing non-tumor cells (other than NK cells), such as B, T cells, monocytes and/or NKT-like cells was observed. PK data from the dose escalation on 22 subjects dosed between 0.2 and 24 mg/kg show that maximum concentrations (Cₘₐₓ) increased roughly proportionally with dose. The AUC from time of dosing to the last quantifiable time point (AUC₀₋ₜ) showed a more than proportional increase with dose up to 4 mg/kg, but a roughly proportional increase at higher dose levels (DLs). No increase in average AUCs was observed between 16 mg/kg and 24 mg/kg. These observations suggest a clear presence of target mediated drug disposition at DLs below 4 mg/kg and a high degree of target saturation throughout the one-week dosing interval from approximately 4 mg/kg upward. At 16 mg/kg, PK profiles were more consistent between subjects and exposure was better maintained during biweekly dosing compared to lower dose levels. Accumulation of peak concentrations from the first full dose onward was limited at all DLs, suggesting a relatively fast linear clearance. Signs of rising trough concentrations over the course of weekly administration were noted in the PK profiles, indicating a reduction of clearance, potentially due to reduction of CD38 through target cell depletion.

Notably, clearance at DLs between 4 and 24 mg/kg for the present antibody was observed to be faster than observed for prior CD38 antibodies daratumumab (Clemens et al., Immunomodulatory Drug Treatment. Clin Pharmacokinet. 2017 August; 56(8):915-924) and isatuximab (Martin et al., Blood Cancer J. 2019 Mar. 29; 9(4):41) in similar dose ranges. This was especially surprising since clearance for other IgGs with similar hexamerization enhancing mutations had previously been observed to be similar as for regular IgGs in mice (De Jong et al., PLoS Biol. 2016 Jan. 6; 14(1):e1002344.)

These results were confirmed in the expansion part A of the trial, where subjects with RRMM who were anti-CD38 mAb-naïve were treated at the RP2D identified for RRMM from the dose escalation part of the trial (16 mg/kg).

The best overall responses among the 11 subjects were complete response in 1 subject (9.1%), very good partial response in 2 subjects (18.2)%, partial response in 3 subjects (27.3%), minimal response in 2 subjects (18.2%), stable disease in 1 subject (9.1%), while 2 were not evaluable.

The most common treatment-emergent AEs (TEAEs) in the expansion phase were neutropenia (6 subjects; 54.5%); and anaemia, headache, IRR, thrombocytopenia, and upper respiratory tract infection (3 subjects per each event; 27.3%).

IRRs (27.3%) were grade 2, manageable, and did not lead to treatment discontinuation. No cytokine release syndrome events were reported.

Treatment was associated with a transient reduction in complement component C2 and total complement lytic activity in the majority of patients, suggesting CDC activity. T cells transiently decreased after administration of the first dose in all patients, and T-cell expansion (≥50% increase from baseline for 2 visits) was observed in 4 of 10 evaluable patients.

Also PK data from the expansion phase confirm what was observed during the dose escalation. The PK profiles receiving 16 mg/kg, is similar between the 2 cohorts. The peak and predose concentrations are comparable between the cohorts.

Thus, the antibody of the invention, when used in humans in the treatment of e.g. hematological malignancies such as MM, surprisingly shows low and manageable side effects and at the same time signs of enhanced antitumor activity (e.g. responses and complement activation).

### Dosages and Treatment Regimens

As described above, the present invention concerns anti-CD38 antibodies, particularly those comprising an Fc region comprising a mutation in one or more amino acid residues selected from the group corresponding to E430, E345 and S440 in a human IgG1 heavy chain. In particular, described herein is the use of such of such antibodies for the treatment of one or more hematological malignancies (e.g. MM), in a subject.

Thus, in a first aspect the present invention provides a method of treating or preventing a hematological malignancy, preferably a multiple myeloma (MM), in a subject in need thereof, preferably a human subject, comprising administering to said subject, an antibody or a pharmaceutical composition comprising the antibody in a therapeutically effective amount, said antibody comprising:
a. an antigen-binding region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4, a VL CDR1 having the sequence as set forth in SEQ ID NO:6, a VL CDR2 having the sequence AAS, and a VL CDR3 having the sequence as set forth in SEQ ID NO:7, and
a. an Fc region comprising a mutation in one or more amino acid residues selected from the group corresponding to E430, E345 and S440 in a human IgG1 heavy chain, wherein the amino acid residues are numbered according to the EU index.

In another aspect, the invention provides method of treating or preventing a hematological malignancy, preferably a multiple myeloma (MM), in a subject in need thereof, preferably a human subject, comprising administering to said subject, an antibody or a pharmaceutical composition comprising the antibody in a therapeutically effective amount, said antibody comprising:
a. a heavy chain comprising a VH region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4 and a human IgG1 CH region with a mutation in one or more of E430, E345 and S440, the amino acid residues being numbered according to the EU index; and
b. a light chain comprising a VL region comprising a VL CDR1 having the sequence as set forth in SEQ ID NO:6, a VL CDR2 having the sequence AAS, and a VL CDR3 having the sequence as set forth in SEQ ID NO:7

In preferred embodiments, the amount of antibody administered (in each dose and/or in each treatment cycle) is at least (about) 4 mg/kg body weight, e.g. the antibody is administered at a dose of at least (about) 4 mg/kg body weight to (about) 24 mg/kg.

In one embodiment, the dose is in the range of between about 4 mg/kg to about 24 mg/kg body weight.

In one embodiment, the dose is in the range of between about 4 mg/kg to about 20 mg/kg body weight.

In one embodiment, the dose is in the range of between about 4 mg/kg to about 16 mg/kg body weight.

In one embodiment, the dose is in the range of between about 4 mg/kg to about 12 mg/kg body weight.

In one embodiment, the dose is in the range of between about 4 mg/kg to about 8 mg/kg body weight.

In one embodiment, the dose is in the range of between about 8 mg/kg to about 24 mg/kg body weight.

In one embodiment, the dose is in the range of between about 8 mg/kg to about 20 mg/kg body weight.

In one embodiment, the dose is in the range of between about 8 mg/kg to about 16 mg/kg body weight.

In one embodiment, the dose is in the range of between about 12 mg/kg to about 24 mg/kg body weight.

In one embodiment, the dose is in the range of between about 12 mg/kg to about 20 mg/kg body weight.

In one embodiment, the dose is in the range of between about 12 mg/kg to about 16 mg/kg body weight.

In one embodiment, the dose is about 4 mg/kg body weight.

In one embodiment, the dose is about 6 mg/kg body weight.

In one embodiment, the dose is about 8 mg/kg body weight.

In one embodiment, the dose is about 10 mg/kg body weight.

In one embodiment, the dose is about 12 mg/kg body weight.

In one embodiment, the dose is about 14 mg/kg body weight.

In one embodiment, the dose is about 16 mg/kg body weight.

In one embodiment, the dose is about 18 mg/kg body weight.

In one embodiment, the dose is about 20 mg/kg body weight.

In one embodiment, the dose is about 22 mg/kg body weight. In one embodiment, the dose is about 24 mg/kg body weight.

In one embodiment, the dose is in the range of between 4 mg/kg to 24 mg/kg body weight.

In one embodiment, the dose is in the range of between 4 mg/kg to 20 mg/kg body weight.

In one embodiment, the dose is in the range of between 4 mg/kg to 16 mg/kg body weight.

In one embodiment, the dose is in the range of between 4 mg/kg to 12 mg/kg body weight.

In one embodiment, the dose is in the range of between 4 mg/kg to 8 mg/kg body weight.

In one embodiment, the dose is in the range of between 8 mg/kg to 24 mg/kg body weight.

In one embodiment, the dose is in the range of between 8 mg/kg to 20 mg/kg body weight.

In one embodiment, the dose is in the range of between 8 mg/kg to 16 mg/kg body weight.

In one embodiment, the dose is in the range of between 12 mg/kg to 24 mg/kg body weight.

In one embodiment, the dose is in the range of between 12 mg/kg to 20 mg/kg body weight.

In one embodiment, the dose is in the range of between 12 mg/kg to 16 mg/kg body weight.

In one embodiment, the dose is 4 mg/kg body weight.

In one embodiment, the dose is 6 mg/kg body weight.

In one embodiment, the dose is 8 mg/kg body weight.

In one embodiment, the dose is 10 mg/kg body weight.

In one embodiment, the dose is 12 mg/kg body weight.

In one embodiment, the dose is 14 mg/kg body weight.

In one embodiment, the dose is 16 mg/kg body weight.

In one embodiment, the dose is 18 mg/kg body weight.

In one embodiment, the dose is 20 mg/kg body weight.

In one embodiment, the dose is 22 mg/kg body weight.

In one embodiment, the dose is 24 mg/kg body weight.

At higher DLs (8 mg/kg and above), PK profiles (exposure) were found to be more consistent among subjects. Thus, in one embodiment, the antibody is administered at a dose of between about 8 mg/kg body weight to about 24 mg/kg body weight, such as between 8 and 24 mg/kg body weight.

Further, no increase in exposure (AUC₀₋ₜ) was observed between 16 and 24 mg/kg. Thus, in one embodiment, the antibody is administered at a dose of between about 8 mg/kg body weight to about 16 mg/kg body weight, such as between 8 and 16 mg/kg body weight. For example, the antibody is administered at a dose of (about) 8 mg/kg body weight or (about) 10 mg/kg body weight, or (about) 12 mg/kg body weight, or (about) 14 mg/kg body weight, or (about) 16 mg/kg body weight.

In a preferred embodiment, the antibody is administered at a dose of about 16 mg/kg body weight or 16 mg/kg body weight. This dose was surprisingly found to be optimal in view of on the one hand the relatively high binding affinity as well as CDC activity observed in vitro and complement activation observed in vivo, and on the other hand the unexpected higher clearance of the antibody across all dose levels, compared to a typical IgG1 antibody or compared to prior art CD38 antibodies. At the same time, no tumor lysis syndrome (TLS) or cytokine release syndrome (CRS) was observed up and EAs were manageable at all dose levels including 16 mg/kg. Further, exposure was found to be maintained better at 16 mg/kg compared to 8 mg/kg, especially when reducing the dosing interval, e.g. during biweekly dosing.

In any of the embodiments as described herein, the dose defined in mg/kg body weight is preferably based on the subject's weigh at the time of administration of each dose, preferably measured within 72 h before administration.

According to any of these embodiments, the dose defined in mg/kg may be converted to flat dose, and vice versa, based on the median body weight of the subjects to whom the binding agent is administered being 70 kg. Thus, the antibody may be administered at a dose of (about) 250-2000 mg, such as (about 280-1700 mg). Any of the above dosages per kg of body weight can in this way thus be converted to a flat rate.

Each treatment cycle treatment preferably is (about) four weeks, i.e. (about) 28 days.

In one embodiment, said dose is administered weekly (Q1W), and preferably said dose is administered at least 1 time, such as 2 times, 3 times, 4 times, 5 times, 6 times, 7 times, more preferably 8 times. Such a weekly dose corresponds to 4 doses in each cycle of about 4 weeks/about 28 days. Thus, preferably, said weekly administration is done for 2 cycles of (about) 4 weeks/(about) 28 days.

In one embodiment, after said weekly administration, one may reduce the interval of administration to an administration once every two weeks (Q2W), also referred to as biweekly administration. Such biweekly administration may preferably be performed at least 1 times, such as 2 times, 3 times, 4 times, 5 times, 6 times, 7 times, more preferably 8 times. Such a biweekly dose corresponds to 2 doses in each cycle of (about) 4 weeks/(about) 28 days. Thus, preferably, said biweekly administration is done for 4 cycles of (about) 4 weeks/(about) 28 days, i.e. is performed 8 times (after said weekly administration).

After said biweekly administration, the interval may be reduced further to once every four weeks (Q4W), i.e. once every (about) 28 days. Such administration every four weeks may be performed for an extended period, preferably at least 1 time, 2 times, 3 times, 4 times, 5 times, 6 times, 7 times, 8 times, 9 times 10 times or even more. Such a dose every four weeks corresponds to 1 dose in each cycle of (about) 4 weeks/(about) 28 days. Thus, preferably said administration every four weeks is done for a period of at least 1 cycle, 2 cycles, 3 cycles, 4 cycles, 5 cycles, 6 cycles, or even more, such as 8 cycles, 10 cycles, 12 cycles, 14 cycles, 16 cycles, 18 cycles, 20 cycles, 24 cycles or even more (after said weekly administration, preferably after said biweekly administration).

In one embodiment, the first dose (or starting dose) of said antibody is administered as a split dose over 2 days, preferably over 2 consecutive days, meaning that 1 part of the first dose is administered on one day and the rest is administered on another day, preferably on the first (C1D1) and second day (C1D2) of the first cycle. Preferably, said split is (about) equal. For example, when the dose is (about) 4 mg/kg body weight, the first dose is administered to said subject as (about) 2 mg/kg on day 1 of cycle 1 (C1D1), and (about) 2 mg/kg on a day shortly thereafter, preferably the next day (i.e. day 2 of cycle 1: C1D2). Likewise, when the dose is (about) 8 mg/kg body weight, the first dose is administered to said subject at (about) 4 mg/kg on day 1 of cycle 1 (C1D1), and (about) 4 mg/kg on a day shortly thereafter, preferably the next day (C1 D2). Likewise, when the dose is (about) 16 mg/kg body weight, the first dose is administered to said subject at (about) 8 mg/kg on day 1 of cycle 1 (C1D1), and (about) 8 mg/kg on a day shortly thereafter, preferably the next day (C1D2). Likewise, when the dose is (about) 24 mg/kg body weight, the first dose is administered to said subject at (about) 12 mg/kg on day 1 of cycle 1 (C1D1), and (about) 12 mg/kg on a day shortly thereafter, preferably the next day (C1D2). Thus, preferably there is (about) 24 h between said first part of the first (split) dose and the second part of said first (split) dose.

In a preferred embodiment, said antibody is administered in cycles of (about) 28 days, i.e. (about) 4 weeks, wherein said antibody is administered weekly (Q1 W) in cycles 1 and 2, every two weeks (Q2W) in cycles 3 through 6, and every 4 weeks (Q4W) as of cycle 7, preferably wherein the first dose is administered as an (equally) split dose over the 1^{st} 2 days.

In one embodiment, the antibody is administered for a time sufficient to treat the hematological malignancy.

In another embodiment, the antibody is administered until disease progression or lack of patient benefit.

In one embodiment, the antibody is administered to said subject by intravenous injection or infusion.

In one embodiment, said antibody is administered by intravenous injection or infusion in a volume of (about) 100 ml to about 500 ml per dose, such as (about) 100 ml or (about) 500 ml per dose.

In one embodiment, said antibody is administered by intravenous injection or infusion for a period of about 1 to (about) 11 hours, or about 1 to (about) 10 hours, or (about) 3 to (about) 10 hours per dose, such as (about) 1 to (about 8) hours, (about) 1 to (about) 5 hours, e.g. (about) 4 hours per dose.

### Diseases and Prior Treatments

The antibody of the present invention, or a pharmaceutical composition comprising the antibody, may be for the use of treatment or prevention of a hematological malignancy or disease or disorder. A “hematological malignancy”, as used herein, refers to a cancer that begins in blood-forming tissue, such as the bone marrow, or in the cells of the immune system. Examples of hematologic cancer are leukemia, lymphoma, and multiple myeloma. A hematological malignancy is sometimes also referred to as blood cancer.

In one embodiment, said disease or disorder may be any hematological disease or disorder as described herein involving cells expressing CD38. In other words, the hematological malignancy may be a CD38 positive hematological malignancy, i.e. a hematological malignancy characterized by the presence of at least part of the tumor cells expressing CD38, including leukemias, lymphomas and myeloma, or a hematological malignancies that is known to express CD38. Examples of such CD38-positive hematological malignancies or hematological malignancies known to express CD38 include precursor B-cell lymphoblastic leukemia/lymphoma and B-cell non-Hodgkin's lymphoma, acute promyelocytic leukemia, acute lymphoblastic leukemia and mature B cell neoplasms, such as B-cell chronic lymphocytic leukemia(CLL)/small lymphocytic lymphoma (SLL), B-cell acute lymphocytic leukemia, B-cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B-cell lymphoma (MALT type, nodal and splenic type), hairy cell leukemia, diffuse large B-cell lymphoma (DLBCL), Burkitt's lymphoma (BL), plasmacytoma, multiple myeloma, plasma cell leukemia, post-transplant lymphoproliferative disorder, light chain amyloidosis, Waldenstrom's macroglobulinemia, plasma cell leukemias and anaplastic large-cell lymphoma (ALCL).

The antibody of the present invention may be for the use of treatment or prevention of a hematological malignancy or disorder, in a subject who has received at least one prior therapy for the same disease or disorder with one or more compounds, wherein said one or more compounds are different from the antibody of the present invention.

For example, in some embodiments, the antibody of the present invention may be for the use of treatment or prevention of a disease or disorder in a subject who have received a prior treatment with a proteasome inhibitor (PI) and/or an immunomodulatory drug (IMiD). Examples of proteasome inhibitors include but are not limited to bortezomib, carfilzomib and ixazomib. Examples of IMiDs include but are not limited to thalidomide, lenalidomide and pomalidomide. In a further embodiment said disease or disorder may be a cancer or a tumor, such as multiple myeloma, mantle cell lymphoma or myelodysplastic syndrome (MDS). Thus, the subject may be a cancer patient, such as a multiple myeloma, mantle cell lymphoma or myelodysplastic syndrome (MDS) patient.

The antibody of the present invention may be for the use of treatment or prevention of a disease or disorder in a subject which have not had any prior treatment with an anti-CD38 antibody. Typically, such a subject or patient is referred to as an anti-CD38 antibody naïve patient. In one embodiment the anti-CD38 antibody is daratumumab and/or isatuximab, i.e. the subject or patient has not had any prior treatment with daratumumab and/or isatuximab. Thus, in one embodiment the subject or patient is a daratumumab-naïve subject/patient or an isatuximab-naive subject/patients.

The present invention also provides the antibodies according to the invention for the use of treatment or prevention of a hematological disorder in a subject who have received at least one prior therapy comprising a CD38 antibody. Such a prior therapy may have been one or more cycles of a planned treatment program comprising CD38 antibody, such as one or more planned cycles of CD38 antibody as single-agent therapy or in a combination therapy, as well as a sequence of treatments administered in a planned manner. In one embodiment, the prior therapy was CD38 antibody monotherapy. In one embodiment, the prior therapy was a combination therapy comprising a CD38 antibody. For example, the prior therapy may have been CD38 antibody in combination with a proteasome inhibitor (PI) and an immunomodulatory agent. In some embodiments, the CD38 antibody is daratumumab or isatuximab.

In some aspects, the subject may also be one where administration of daratumumab and/or isatuximab as a monotherapy has a limited effect.

In some aspects, the hematological malignancy can be characterized as cancer that is “refractory” or “relapsed” to a prior therapy. In a further embodiment, the prior therapy may comprise one or more of a PI, an IMiD, and a CD38 antibody, e.g. wherein the CD38 antibody is daratumumab or isatuximab. Typically, this indicates that the prior therapy achieved less than a complete response (CR), for example, that the cancer was non-responsive to CD38 antibody mono- or combination therapy or that the cancer progressed within a predetermined period of time after the end of CD38 antibody therapy.

Examples of such combination therapies include, but are not limited to, combination of a CD38 antibody with a PI or an IMiD or a combination of a PI and an IMiD. Similarly, it may indicate that that the prior therapy achieved less than a complete response (CR), for example, that the cancer was non-responsive to a PI, or an IMiD or a combination therapy thereof, or that the cancer progressed within a predetermined period of time after the end of said therapy. The skilled person can determine whether a cancer is refractory to a prior therapy based on what is known in the art, including guidelines available for each cancer.

For example, in multiple myeloma, refractory and relapsed disease can be identified according to the guidelines published by Rajkumar, Harousseau et al., on behalf of the International Myeloma Workshop Consensus Panel, Consensus recommendations for the uniform reporting of clinical trials: report of the International Myeloma Workshop Consensus Panel, Blood 2011.117:4691-4695:

Refractory myeloma can be defined as disease that is nonresponsive while on primary or salvage therapy, or progresses within 60 days of last therapy. Nonresponsive disease is defined as either failure to achieve minimal response or development of progressive disease (PD) while on therapy. There may be 2 categories of refractory myeloma: “relapsed-and-refractory myeloma” and “primary refractory myeloma”:

Relapsed and refractory myeloma can be defined as disease that is nonresponsive while on salvage therapy, or progresses within 60 days of last therapy in patients who have achieved minimal response (MR) or better at some point previously before then progressing in their disease course.

Primary refractory myeloma can be defined as disease that is nonresponsive in patients who have never achieved a minimal response or better with any therapy. It includes patients who never achieve MR or better in whom there is no significant change in M protein and no evidence of clinical progression as well as primary refractory, PD where patients meet criteria for true PD. On reporting treatment efficacy for primary refractory patients, the efficacy in these 2 subgroups (“nonresponding-nonprogressive” and “progressive”) should be separately specified.

Relapsed myeloma can be defined as previously treated myeloma that progresses and requires the initiation of salvage therapy but does not meet criteria for either “primary refractory myeloma” or “relapsed-and-refractory myeloma” categories.

For details on specific responses (CR, PR etc.) in multiple myeloma and how to test them, see Rajkumar, Harousseau et al., 2011 (supra).

Accordingly, in some embodiments, the antibody according to any aspect or embodiment herein, or a pharmaceutical composition comprising the antibody, is for use in treating a cancer which is refractory to a prior treatment comprising one or more of a PI, an IMiD and a CD38 antibody. In one embodiment the prior treatment comprises a CD38 antibody. In a specific embodiment, the cancer is identified as a refractory cancer before the use.

In another embodiment, there is provided for a method for treating cancer in a subject, comprising the steps of:
1. identifying the subject as being refractory to a prior treatment comprising one or more of a PI, an IMiD and a CD38 antibody, and
2. administering a therapeutically effective amount of the antibody according to any aspect or embodiment herein, or a pharmaceutical composition comprising the antibody variant to the subject.

In one embodiment the prior treatment comprises a CD38 antibody.

In another embodiment, there is provided for a method for treating cancer refractory to a prior treatment comprising one or more of a PI, an IMiD and a CD38 antibody in a subject, comprising administering a therapeutically effective amount of the antibody according to any aspect or embodiment herein, or a pharmaceutical composition comprising the antibody to the subject. In one embodiment the prior treatment comprises a CD38 antibody.

In some embodiments the PI is selected from the group consisting of bortezomib, carfilzomib and ixazomib.

In some embodiments the IMiD is selected from the group consisting of thalidomide, lenalidomide and pomalidomide.

In some embodiments, the CD38 antibody is daratumumab. In some embodiments, the CD38 antibody is isatuximab. In some embodiments, the CD38 antibody may also be felzartamab or mezagitamab.

In some embodiments, the antibody according to any aspect or embodiment herein, or a pharmaceutical composition comprising the antibody, is for use in treating a cancer which is relapsed after a prior treatment comprising one or more of a PI, an IMiD and a CD38 antibody. In one embodiment the prior treatment comprises a CD38 antibody. In a specific embodiment, the cancer is identified as relapsed before the use.

In another embodiment, there is provided for a method for treating cancer in a subject, comprising the steps of:
1. identifying the subject as being relapsed after a prior treatment comprising one or more of a PI, an IMiD and a CD38 antibody, and
2. administering a therapeutically effective amount of the antibody according to any aspect or embodiment herein, or a pharmaceutical composition comprising the antibody to the subject.

In one embodiment the prior treatment comprises a CD38 antibody.

In another embodiment, there is provided for a method for treating cancer relapsed after a prior treatment comprising one or more of a PI, an IMiD and a CD38 antibody in a subject, comprising administering a therapeutically effective amount of the antibody according to any aspect or embodiment herein, or a pharmaceutical composition comprising the antibody to the subject. In one embodiment the prior treatment comprises a CD38 antibody.

In some embodiments the PI is selected from the group consisting of bortezomib, carfilzomib and ixazomib.

In some embodiments the IMiD is selected from the group consisting of thalidomide, lenalidomide and pomalidomide.

In some embodiments, the CD38 antibody is daratumumab. In some embodiments, the CD38 antibody is isatuximab. In some embodiments, the CD38 antibody may also be felzartamab or mezagitamab.

In specific embodiments, the antibody according to the present invention is administered in a therapeutically effective amount and/or for a sufficient period of time to treat the refractory or relapsed cancer.

In some embodiments of any of the methods and uses as described herein, the refractory or relapsed cancer is a hematological cancer.

In some embodiments, the refractory or relapsed cancer is selected from the group consisting of multiple myeloma (MM), chronic lymphocytic leukemia (CLL), acute lymphoblastic leukemia (ALL), mantle cell lymphoma (MCL), follicular lymphoma (FL), and diffuse large B-cell lymphoma (DLBCL).

In some embodiments, the refractory or relapsed cancer is selected from the group consisting of multiple myeloma (MM), chronic lymphocytic leukemia (CLL), mantle cell lymphoma (MCL), diffuse large B-cell lymphoma (DLBCL), and follicular lymphoma (FL).

In some embodiments, the refractory or relapsed cancer is chronic lymphocytic leukemia (CLL).

In some embodiments, the refractory or relapsed cancer is mantle cell lymphoma (MCL).

In some embodiments, the refractory or relapsed cancer is diffuse large B-cell lymphoma (DLBCL).

In some embodiments, the refractory or relapsed cancer is follicular lymphoma (FL).

In some especially preferred embodiments, the refractory or relapsed cancer is multiple myeloma (MM).

In some embodiments, relapsed or refractory multiple myeloma is characterized by evidence of disease progression in said subject on the most recent prior treatment regimen based on IMWG 2016 criteria with measurable disease. Said criteria may be selected from the group consisting of:
a. Prior documentation of monoclonal plasma cells in the bone marrow ≥10% or presence of a biopsy-proven plasmacytoma
b. IgG, IgA, IgD, or IgM myeloma: Serum M-protein level ≥0.5 g/dL (≥5 g/L) or urine M protein level ≥200 mg/24 hours;
c. Light chain myeloma: Serum Ig free light chain (FLC) ≥10 mg/dL and abnormal serum Ig kappa lambda FLC ratio

In one embodiment, the criteria are a. in combination with b. or c. as indicated above.

In some preferred embodiments according to any of the methods and uses herein, the hematological malignancy is diffuse large B cell lymphoma (DLBLC), such as relapsed or refractory DLBCL. Thus, in another aspect, the method in accordance with the invention as provided for any of the embodiments described herein, comprises the treatment of DLBCL in a (human) subject in need thereof, such as refractory or relapsed DLBLC, for example DLBCL that is relapsed or refractory to a prior a-CD38 treatment (e.g. daratumumab or isatuximab). The dosages and treatment cycles can be as described elsewhere herein. Thus, in any of these embodiments, the amount of antibody administered (in each dose and/or in each treatment cycle) is at least (about) 4 mg/kg body weight, such as between 4 mg/kg and 24 mg/kg body weight. Preferably, the antibody is administered at a dose of (about) 8 mg/kg body weight to (about) 24 mg/kg, or at a dose of (about) 8 mg/kg body weight to (about) 16 mg/kg body weight. In one embodiment in the treatment of DLBCL, the antibody is administered at a dose of (about) 4 mg/kg body weight. In another embodiment, the antibody is administered at a dose of (about) 8 mg/kg body weight. In yet another embodiment, the antibody is administered at a dose of (about) 16 mg/kg body weight.

In one embodiment, relapsed or refractory DLBCL includes both de novo or histologically transformed. Relapsed disease can be defined as the reappearance or growth of lymphoma after at least 6 months duration of response (DOR). Refractory disease can be defined as failure to achieve response after at least 2 cycles of therapy or reappearance after a DOR of <6 months.

Efficacy assessment for DLBCL of the treatment may be conducted in accordance with the Lugano response criteria for lymphoma (Cheson et al., 2014), as shown in table 2 and 3 below

TABLE 2
Lugano Response Criteria DLBCL - CT/MRI Scan

| | | Non- | | | |
|---|---|---|---|---|---|
| | | measurable | Organ | | |
| Response | Lymph Nodes | Lesion | Enlargement | New Lesions | Bone Marrow |
| Complete | Target nodes/nodal masses | Absent | Regress to | None | Normal by |
| Response | regression ≤1.5 cm in | | normal | | morphology |
| (CR) | longest diameter | | | | |
| Partial | 50% decrease in SPD of up | Absent/normal, | Spleen | None | Not applicable |
| Response | to 6 target measurable | regressed, | regressed >50% | | |
| (PR) | nodes and extranodal sites | but no | in length | | |
| | | increase | beyond normal | | |
| | | | (13 cm) | | |
| No response | <50% decrease in SPD of | No increase | No increase | None | Not applicable |
| or Stable | up to 6 target measurable | consistent | consistent with | | |
| Disease (SD) | nodes and extranodal sites | with | progression | | |
| | | progression | | | |
| Progressive | >1.5 cm and increase by | New or | Spleen length | New nodes >1.5 cm | New or |
| disease (PD) | 50% from nadir and | clear | increase | New extranodal | recurrent |
| | increase from nadir | progression | of >50% | site >1.0 cm | involvement |
| | 1. 0.5 cm for | of pre- | | Assessable disease of | |
| | lesions ≤2 cm | existing | | any size attributable to | |
| | 2. 1.0 cm for | lesions | | lymphoma | |
| | lesions >2 cm | | | | |

TABLE 3
Lugano Response Criteria DLBCL - PET/CT Scan

| | | Non- | | | |
|---|---|---|---|---|---|
| | | measurable | Organ | | |
| Response | Lymph Nodes | Lesion | Enlargement | New Lesions | Bone Marrow |
| Complete | Score 1, 2 or 3 with or | Not | Not applicable | None | No evidence of FDG-avid |
| Response | without a residual mass on | applicable | | | disease in marrow |
| (CR) | 5PS^{a} | | | | |
| Partial | Score 4 or 5 with reduced | Not | Not applicable | None | Residual uptake higher |
| Response | uptake compared to | applicable | | | than uptake in normal |
| (PR) | baseline and residual mass | | | | marrow but reduced |
| | of any size | | | | compared to baseline |
| No response | Score 4 or 5 with no | Not | Not applicable | None | No change from baseline |
| or Stable | significant change in FDG | applicable | | | |
| Disease (SD) | uptake | | | | |
| Progressive | Score 4 or 5 with an increase | None | | New FDG- | New or recurrent FDG- |
| disease (PD) | in intensity of uptake | | | avid foci | avid foci |
| | | | | consistent | |
| | | | | with | |
| | | | | lymphoma | |

^{a}PET 5PS: 1, no uptake above background; 2, uptake ≤ mediastinum; 3, uptake > mediastinum but ≤ liver; 4, uptake moderately > liver; 5, uptake markedly higher than liver and/or new lesions; X, new areas of uptake unlikely to be related to lymphoma.

### Therapeutic Effects

### Responses

In any of the methods and uses as described herein, the administration of the antibody according to the invention may induce one or more therapeutic effects in in the treated subject, and/or may improve one or more therapeutic effects in the treated subject relative to a baseline.

Thus, in some embodiments, the one or more therapeutic effects that is induced and/or improved is selected from the group consisting of overall response rate, duration of response, time to response.

In some embodiments, therapeutic effect is a stringent complete response, complete response, very good partial response, partial response, minimal response or stable disease status. Optionally, the treatment can be continued until disease progression or lack of patient benefit.

In one embodiment, the therapeutic effect is a stringent complete response.

In one embodiment, the therapeutic effect is a complete response.

In one embodiment, the therapeutic effect is a very good partial response.

In one embodiment, the therapeutic effect is a partial response.

In one embodiment, the therapeutic effect is a minimal response.

In one embodiment, the therapeutic effect is stable disease status.

In one embodiment, where said hematological malignancy preferably is (relapsed or refractory) multiple myeloma, wherein the therapeutic effect is an overall response rate of at least (about) 14%, such as at least (about) 16%, 18%, 20%, 22%, 24%, 26%, 28%, 30%, 35%, 40% or more in the treated subjects, optionally wherein said antibody is administered at a dose of at least (about) 4 mg/kg, such as between (about) 4 and 24 mg/kg.

In some embodiments, wherein said hematological malignancy is a cancer, preferably (relapsed or refractory) multiple myeloma, that has not been previously treated with a prior therapy comprising a CD38 antibody, preferably daratumumab or isatuximab, the therapeutic effect is an overall response rate at least (about) 20%, such as at least (about) 25%, 30%, 35%, 40%, 45%, 50%, 55%, 60%, 65%, 70% or more in the treated subjects, optionally wherein said antibody is administered at a dose of (about) at a dose of at least (about) 4 mg/kg, such as between (about) 4 and 24 mg/kg, such as (about) 16 mg/kg.

In some embodiments, where the hematological malignancy is a cancer, preferably (relapsed or refractory) multiple myeloma, that is relapsed or refractory to a prior anti-cancer therapy, such as a prior therapy comprising a CD38 antibody, preferably daratumumab or isatuximab, the therapeutic effect is an overall response rate at least (about) 6%, such as at least (about) 8%, 10%, 12%, 14%, 16%, 18%, 20%, 25%, 30%, 35%, 40% or more in the treated subjects, optionally wherein said antibody is administered at a dose of (about) at a dose of at least (about) 4 mg/kg, such as between (about) 4 and 24 mg/kg, such as (about) 16 mg/kg.

In certain preferred embodiments, wherein said hematological malignancy is a cancer, preferably (relapsed or refractory) multiple myeloma, that has not been previously treated with a prior therapy comprising a CD38 antibody, preferably daratumumab or isatuximab, the therapeutic effect is at least (about) 25 very good partial responses (VGPRs) in the treated subjects, such as at least (about) 30%, 35%, 40%, 45%, 50%, 60% 70% or more VGPRs or better in the treated subjects, optionally wherein said dose is at least (about) 4 mg/kg body weight or alternatively at least (about) 8 mg/kg body weight or at least (about) 16 mg/kg body weight or at least (about) 24 mg/kg body weight. In certain embodiments, the therapeutic effect is at least (about) 9% CRs in the treated subjects, such as at least (about) 10%, 15%, 20%, 24%, 30%, 35%, 40%, 45%, 50%, 60% 70% or more CRs, optionally wherein said dose is at least (about) 4 mg/kg body weight or alternatively at least (about) 8 mg/kg body weight or at least (about) 16 mg/kg body weight or at least (about) 24 mg/kg body weight.

In some embodiments, where the hematological malignancy is a cancer that is relapsed or refractory to a prior anti-cancer therapy, such as a prior therapy comprising a CD38 antibody, preferably daratumumab or isatuximab, the therapeutic effect is at least 6% partial responses (PRs) in the treated subjects, such as at least 8%, 10%, 12%, 14%, 15%, 16%, 17%, 18%, 19%, 20%, 21%, 22%, 23%, 24%, 25% or more PRs in the treated subjects, optionally wherein said dose is at least about 4 mg/kg body weight or alternatively at least about 8 mg/kg body weight or at least about 16 mg/kg body weight or at least about 24 mg/kg body weight, preferably at least 16 mg/kg body weight.

In one embodiment, the antibody when used according to any aspect or embodiment disclosed herein, improves said one or more therapeutic effects in said subject relative to a treatment with a control antibody (at a similar or comparable or equivalent dose. The control can be, for example, a reference antibody with amino acid sequences (typically heavy- and light chain amino acid sequences) identical to the antibody of the invention except for the one or more mutations in E430, E345 and/or S440. In a preferred embodiment, the antibody of the invention comprises a mutation at position E430, preferably E340G (in a human IgG1 heavy chain), and said reference antibody does not comprise said mutation at position E430 (i.e. is wt at said position), preferably comprises a wt CH3/Fc region (in a human IgG1 heavy chain).

In another embodiment, the control is a reference antibody with amino acid sequences (typically heavy- and light chain amino acid sequences) identical to the antibody of the invention except for different VH and VL sequences.

In one embodiment, the antibody when used according to any aspect or embodiment disclosed herein, improves said one or more therapeutic effects in said subject relative to a reference antibody, wherein the reference antibody comprises the VH and VL region sequences of antibody C, i.e., SEQ ID NO:1 and SEQ ID NO:5, respectively, and CH and CL region sequences identical to the antibody of the invention except for the one or more mutations in E430, E345 and/or S440. In a preferred embodiment, the antibody of the invention comprises a mutation at position E430 (in a human IgG1 heavy chain), preferably E340G, and said reference antibody does not comprise said mutation at position E430. (i.e. is wt at said position), preferably comprises a wt CH3/Fc region (in a human IgG1 heavy chain).

In some embodiments, the antibody when used according to any aspect or embodiment disclosed herein, improves said one or more therapeutic effects in said subject relative to a reference antibody, wherein the reference antibody comprises the VH and VL region sequences of antibody B, i.e., SEQ ID NO:8 and SEQ ID NO:9, respectively and CH and CL region sequences identical to the antibody of the invention.

In one embodiment, the antibody when used according to any aspect or embodiment disclosed herein, improves said one or more therapeutic effects in said subject relative to a reference antibody, wherein the reference antibody comprises the VH and VL region sequences of antibody B, i.e., SEQ ID NO:8 and SEQ ID NO:9, respectively, and the CH and CL region sequences of SEQ ID NO:20 (IgGm(f)) and SEQ ID NO:37 (kappa), respectively, or wherein the reference antibody is antibody B or daratumumab or isatuximab. In a preferred embodiment, the antibody of the invention comprises a mutation at position E430 (in a human IgG1 heavy chain), preferably E340G.

The reference antibody may also be felzartamab or mezagitamab.

In one embodiment, said one or more therapeutic effects may be achieved when the antibody is administered at a dose of at least (about) 4 mg/kg body weight, such as between (about) 4 mg and (about) 24 mg/kg body weight, or any intermediate dose level or range as described herein.

In one embodiment, said one or more therapeutic effects may be achieved at a dose of at least (about) 8 mg/kg body weight, such as between (about) 8 and (about) 24 mg/kg body weight or between (about) 8 and (about) 16 mg/kg body weight.

In one embodiment, said one or more therapeutic effects may be achieved at a dose of (about) 4 mg/kg body weight.

In one embodiment, said one or more therapeutic effects may be achieved at a dose of (about) 8 mg/kg body weight.

In one embodiment, said one or more therapeutic effects may be achieved at a dose of (about) 16 mg/kg body weight.

In one embodiment, said one or more therapeutic effects may be achieved at a dose of (about) 24 mg/kg body weight.

In one embodiment, said one or more therapeutic effects may be achieved when the antibody is administered to a subject has not previously been treated with an anti-CD38 antibody, such as daratumumab, isatuximab, felzartamab, and mezagitamab (an anti-CD38 mAb-naïve subject)

In one embodiment, said one or more therapeutic effects may be achieved when the antibody is administered to a subject that has previously been treated with an anti-CD38 antibody, e.g. a subject wherein the hematological malignancy is a cancer that is relapsed or refractory after a prior therapy comprising an anti-CD38 antibody (an anti-CD38 pretreated subject), such as such as daratumumab, isatuximab, felzartamab, and mezagitamab.

### Management of Side Effects

In some embodiments according any of the methods and uses herein, the subject may be treated for the management of infusion related reaction (IRRs). For this, pre-infusion medication (e.g. corticosteroids, antipyretics, antihistamines, a leukotriene receptor antagonist) and/or post-infusion medication (e.g. corticosteroids) may be given to reduce the risk of or for the treatment of IRRs, especially IRR of grade 2 or higher. Said pre-infusion medication may be administered about 1-3 hours prior to said administration of said antibody and/or said post-infusion medication may be administered on the two days following said administration of said antibody.

The pre-infusion medication may comprise corticosteroids (e.g methylprednisolone, betametasone, dexamethasone, triamcinolone, prednisone and/or prednisolone), antihistamines (e.g. diphenhydramine), antipyretics (e.g. paracetamol) and/or a leukotriene receptor antagonist (e.g. montelukast), optionally wherein
a. said corticosteroid is administered at a dose of about 60-100 mg methylprednisolone or equivalent;
b. Said diphenhydramine is administered at a dose of about 25-50 mg;
c. said paracetamol is administered at a dose of about 650-1000 mg; and/or
d. said montelukast is administered at a dose of about 10 mg 10 mg.

The post-infusion medication may comprise corticosteroids, e.g methylprednisolone, betametasone, dexamethasone, triamcinolone, prednisone and/or prednisolone, optionally wherein said corticosteroid is administered at a dose of 20 mg methylprednisolone or equivalent.

In some embodiments according any of the methods and uses herein, the subjects may be treated for the management of cytopenias, for example neutropenia or thrombocytopenia, in particular grade 3 or 4 neutropenia or thrombocytopenia. Such treatment may be with granulocyte colony-stimulating factor (G-CSF) and/or other hematopoetic growth factors (e.g. erythropoetin) and/or transfusion with blood products (e.g. red blood cells and platelet transfusions), preferably G-CSF. In one embodiment, G-CSF is administered when the antibody is administered at a dose level of 4 mg/kg body weight or higher, e.g. when grade 3 or 4 neutropenia is observed.

### Pharmacokinetics

In one embodiment, the antibody when used according to the methods and uses as described herein, displays a higher clearance than a than a reference antibody.

In one embodiment, the reference antibody is a regular IgG antibody, e.g. an IgG1 antibody not comprising a mutation in one or more amino acid residues selected from the group corresponding to E430, E345 and S440 in a human IgG1 heavy chain. In a preferred embodiment, the antibody of the invention comprises a mutation at position E430, preferably E340G, and said reference antibody does not comprise said mutation at position E430 (i.e. is wt at said position).

Thus, in one embodiment, the antibody when used according to the methods and uses as described herein, displays a higher clearance than a than a reference antibody, wherein the reference antibody comprises the VH and VL region sequences of antibody C, i.e., SEQ ID NO:1 and SEQ ID NO:5, respectively, and identical CH and CL region except for the one or more mutations in E430, E345 and/or S440. In a preferred embodiment, the antibody of the invention comprises a mutation at position E430 (in a human IgG1 heavy chain), preferably E340G, and said reference antibody does not comprise said mutation at position E430. (i.e. is wt at said position), preferably comprises a wt CH3/Fc region (in a human IgG1 heavy chain).

In one embodiment, the antibody when used according to the methods and uses as described herein, displays a higher clearance than a reference antibody, wherein the reference antibody is another anti-CD38 antibody not comprising a mutation in one or more amino acid residues selected from the group corresponding to E430, E345 and S440 in a human IgG1 heavy chain. In a preferred embodiment, the antibody of the invention comprises a mutation at position E430 (in a human IgG1 heavy chain), preferably E340G, and said reference antibody does not comprise said mutation at position E430. (i.e. is wt at said position), preferably comprises a wt CH3/Fc region (in a human IgG1 heavy chain)

Thus, in one embodiment, the antibody when used according to the methods and uses as described herein, displays a higher clearance than a reference antibody, wherein the reference antibody comprises the VH and VL region sequences of antibody B, i.e., SEQ ID NO:8 and SEQ ID NO:9, respectively, and the CH and CL region sequences of SEQ ID NO:20 (IgGm(f)) and SEQ ID NO:37 (kappa), respectively, or wherein the reference antibody is antibody B or wherein the reference antibody is daratumumab or isatuximab. In a preferred embodiment, the antibody of the invention comprises a mutation at position E430 (in a human IgG1 heavy chain), preferably E340G.

The reference antibody may also be felzartamab or mezagitamab.

In particular, the higher clearance than a reference antibody may occur when the antibody is administered at a dose of at least (about) 4 mg/kg body weight, such as at (about) 6 mg/kg body weight, such as at (about) 8 mg/kg body weight, such as at (about) 10 mg/kg body weight, such as at (about) 12 mg/kg body weight, such as at (about) 14 mg/kg body weight, such as at (about) 16 mg/kg body weight, such as at (about) 20 mg/kg body weight, such as at (about) 22 mg/kg body weight, such as at (about) 24 mg/kg body weight. At such higher doses, target mediated drug disposition appeared to be saturated.

In one embodiment, said clearance is non-target mediated clearance.

In one embodiment, said higher clearance is FcRn-dependent.

Clearance, as used herein, is defined as the dose divided by the estimated area under the serum concentration-time curve between start of administration and infinity.

### Pharmacodynamics

The antibody when used according to the methods and uses as described herein can typically have one or more of the following effects in the subject: activation of the complement system, depletion of peripheral blood NK cells, expansion of peripheral blood NK cells, or any combination thereof.

The antibody when used according to the methods and uses as described herein typically does not or substantially does not have one or more of the following effects in the subject: a dose dependent increase in plasma levels of proinflammatory cytokines or a dose dependent reduction of certain non-tumor cells other than NK cells (B cells, T cells, monocytes and/or NKT-like cells), or any combinations thereof.

In one embodiment, the antibody when used according to the methods and uses as described herein induces activation of the complement system in the subject. Said activation of the complement system can be a reduction in complement component C2, e.g. as measured in in peripheral blood. Reduction of C2 is indicative of the CDC activity of the antibody of the invention in vivo. The reduction in C2 can be transient after each dose, e.g. returning to baseline before the next dose, typically within 8 days after the dose. In one embodiment, C2 levels can decrease by at least 30% from baseline (mean peak or median peak), such as by at least (about) 35%, 40%, 45%, 50%, 55%, 58%, 60%, 64%, 70%, 75%, 80% or even more.

C2 levels in peripheral blood can e.g. be measured according to any suitable method as known in the art, e.g. essentially according to the method as described in Example 6.

Said activation of the complement system can also be a reduction in the total complement lytic activity (CH50), e.g. as measured in peripheral blood. Reduction of CH50 is indicative of the CDC activity of the antibody of the invention in vivo. The reduction in CH50 can be transient after each dose, e.g. returning to baseline before the next dose, typically within 8 days after the dose. In one embodiment, CH50 levels can decrease by more than 20% from baseline (mean peak or median peak), such as by (about) 25%, 30%, 32%, 35%, 40%, 45%, 48%, 50%, 53%, 55%, 60%, 65%, 70%, 75%, 80%, 85%, 90% or even more.

CH50 levels in peripheral blood can e.g. be measured according to any suitable method as known in the art, e.g. essentially according to the method as described in Example 7.

In some embodiments, the activation of the complement system, such as the reduction in C2 and/or CH50 of the antibody of the invention is greater than a control antibody. The control can be, for example, a reference antibody with amino acid sequences (typically heavy- and light chain amino acid sequences) identical to the antibody except for the one or more mutations in E430, E345 and/or S440. In a preferred embodiment, the antibody of the invention comprises a mutation at position E430 (in a human IgG1 heavy chain), preferably E340G, and said reference antibody does not comprise said mutation at position E430. (i.e. is wt at said position), preferably comprises a wt CH3/Fc region (in a human IgG1 heavy chain). Alternatively, the control can be a reference antibody with amino acid sequences (typically heavy- and light chain amino acid sequences) identical to the antibody of the invention except for different VH and VL sequences. Such a reference antibody could, for example, instead have the VH and VL sequences of antibody B or A, as shown in Table 4. Preferably, the VH and VL sequences of the reference antibody are those of antibody B. Alternatively, the reference antibody may be an antibody binding the same target but with different amino acid sequences.

Accordingly, in one embodiment, the antibody when used according to any aspect or embodiment disclosed, herein induces a higher activation of the complement system, e.g. a greater reduction in C2 and/or CH50, than a reference antibody, wherein the reference antibody comprises the VH and VL region sequences of antibody C, i.e., SEQ ID NO:1 and SEQ ID NO:5, respectively, and CH and CL region sequences identical to the antibody of the invention except for the one or more mutations in E430, E345 and/or S440. In a preferred embodiment, the antibody of the invention comprises a mutation at position E430 (in a human IgG1 heavy chain), preferably E340G, and said reference antibody does not comprise said mutation at position E430. (i.e. is wt at said position), preferably comprises a wt CH3/Fc region (in a human IgG1 heavy chain).

In another embodiment, the antibody when used according to any aspect or embodiment disclosed herein, induces a higher induces a higher activation of the complement system, e.g. a greater reduction in C2 and/or CH50, than a reference antibody, wherein the reference antibody comprises the VH and VL region sequences of antibody C, i.e., SEQ ID NO:1 and SEQ ID NO:5, respectively, and the CH and CL region sequences of SEQ ID NO:20 (IgGm(f)) and SEQ ID NO:37 (kappa), respectively.

In another embodiment, the antibody when used according to any aspect or embodiment disclosed herein, induces a higher activation of the complement system, e.g. a greater reduction in C2 and/or CH50, than a reference antibody, wherein the reference antibody comprises the VH and VL region sequences of antibody B, i.e., SEQ ID NO:8 and SEQ ID NO:9, respectively, and CH and CL region sequences identical to the antibody of the invention.

In one embodiment, the antibody when used according to any aspect or embodiment disclosed herein, induces a higher activation of the complement system, e.g. a greater reduction in C2 and/or CH50, than a reference antibody, wherein the reference antibody comprises the VH and VL region sequences of antibody B, i.e., SEQ ID NO:8 and SEQ ID NO:9, respectively, and the CH and CL region sequences of SEQ ID NO:20 (IgGm(f)) and SEQ ID NO:37 (kappa), respectively, or wherein the reference antibody is antibody B. In a preferred embodiment, the antibody of the invention comprises a mutation at position E430 (in a human IgG1 heavy chain), preferably E340G.

In one embodiment, the antibody when used according to the methods and uses as described herein, does not exhaust the complement system. Thus, the reduction in C2 and/or CH50 preferably is transient, e.g. returning to baseline after a certain time, preferably before the next dose is administered, e.g. within (about) 8 days after dosing. Preferably, the complement system is not exhausted at any dose level between 4 and 24 mg/kg body weight, e.g. it is not exhausted when the antibody is administered at a dose of at (about) 4 mg/kg body weight, at (about) 6 mg/kg body weight, at (about) 8 mg/kg body weight, at (about) 10 mg/kg body weight, at (about) 12 mg/kg body weight, at (about) 14 mg/kg body weight, at (about) 16 mg/kg body weight, at (about) 20 mg/kg body weight, at (about) 22 mg/kg body weight, at (about) 24 mg/kg body weight. Thus, at any of these dose levels, CH2 and/or CH50 levels preferably return to baseline after several days, such as within (about) 8 days.

In another embodiment, the antibody when used according to the methods and uses as described herein, induces depletion of peripheral blood NK cells in the subject. NK cell depletion is indicative of the ADCC activity of the antibody of the invention in vivo. In one embodiment, NK cell deletion is induced when the antibody is administered at a dose level of at least 0.2 mg/kg body weight such at between 0.2 and 24 mg/kg, for example at (about) 4 mg/kg body weight, at (about) 6 mg/kg body weight, at (about) 8 mg/kg body weight, at (about) 10 mg/kg body weight, at (about) 12 mg/kg body weight, at (about) 14 mg/kg body weight, at (about) 16 mg/kg body weight, at (about) 20 mg/kg body weight, at (about) 22 mg/kg body weight, at (about) 24 mg/kg body weight. In one embodiment, NK cells remained reduced or substantially reduced throughout the treatment, e.g. throughout cycle 1, or additionally throughout cycle 2, or additionally throughout cycle 3, or additionally throughout cycle 4, or additionally throughout cycle 5, and optionally beyond.

Peripheral blood NK cell count can e.g. be determined according to any suitable method as known in the art, e.g. essentially according to the method as described in Example 5.

In another embodiment, the antibody when used according to the methods and uses as described herein, induces expansion of peripheral blood T cells in the subject. T cell expansion is indicative of the immunomodulatory activity of the antibody of the invention in vivo (e.g. inhibition of cyclase activity of CD38 or depletion of regulatory cells such as Tregs).

In one embodiment, the subject has not received a prior therapy comprising a CD38 antibody (such as daratumumab and/or isatuximab).

Peripheral blood T cell count can e.g. be determined according to any suitable method as known in the art, e.g. essentially according to the method as described in Example 5.

In another embodiment, the antibody when used according to the methods and uses as described herein, does not induce or does not substantially induce a dose-dependent increase in plasma levels of proinflammatory cytokines, such as IL-2, IL-6, IL-8, IL-10, IFNy and/or TNFa in the subject. In another embodiment, the antibody when used according to the methods and uses as described herein, does not induce or does not substantially induce a dose-dependent increase in plasma levels of proinflammatory cytokines, such as IL-2, IL-6, IL-8, IL-10 and/or IFNy in the subject.

In another embodiment, the antibody when used according to the methods and uses as described herein does not induce or does not substantially induce a dose-dependent reduction in certain CD38-expressing non-tumor cells (other than NK cells), wherein said CD38 expressing non-tumor cells are selected from the group consisting of B cells, T cells, monocytes and/or NKT-like cells, in said subject.

Count of B, T cells, monocytes and NKT-like cells can e.g. be determined according to any suitable method as known in the art, e.g. essentially according to the methods as described in Example 5.

In one embodiment, the antibody when used according to the methods and uses as described herein,
a. has an inhibitory effect on CD38 cyclase activity in said subject;
b. induces complement-dependent cytotoxicity (CDC) of cells expressing human CD38 in said subject;
c. induces antibody-dependent cell-mediated cytotoxicity (ADCC) of cells expressing human CD38 in said subject;
d. induces antibody-dependent cellular phagocytosis (ADCP) of cells expressing human CD38 in said subject;
e. induces apoptosis in the presence of FcgR-bearing cells in said subject;
f. induces trogocytosis of cells expressing human CD38; or
g. any combination of a. to f.

In some embodiments, any one or all of a. b and f. are higher compared to a reference antibody not comprising a mutation in one or more amino acid residues selected from the group corresponding to E430, E345 and S440 in a human IgG1 heavy chain, wherein the amino acid residues are numbered according to the EU index index (when administered at a similar or comparable or equivalent dose).

Suitable assays for evaluating CD38 cyclase activity, CDC, ADCC, ADCP, trogocytosis and apoptosis are known in the art and are e.g. described in WO 2020/012036 A1 and WO 2020/012038 A1.

In one embodiment, the antibody when used according to the methods and uses as described herein induces trogocytosis in said subject, such as trogocytosis of CD38 from donor CD38-expressing cells to acceptor cells. Typical acceptor cells include T and B cells, monocytes/macrophages, dendritic cells, neutrophils, and NK cells. Preferably, the acceptor cells are lymphocytes expressing Fc-gamma-(Fcγ)-receptors, such as, e.g., macrophages or PBMCs. In particular, the antibody of the present invention may mediate an increased trogocytosis as compared to a control. The control can be, for example, a reference antibody with amino acid sequences (typically heavy- and light chain amino acid sequences) identical to the antibody of the invention except for the one or more mutations in E430, E345 and/or S440. In a preferred embodiment, the antibody of the invention comprises a mutation at position E430, preferably E340G (in a human IgG1 heavy chain), and said reference antibody does not comprise said mutation at position E430. (i.e. is wt at said position), preferably comprises a wt CH3/Fc region (in a human IgG1 heavy chain).

In another embodiment, the control is a reference antibody with amino acid sequences (typically heavy- and light chain amino acid sequences) identical to the antibody of the invention except for different VH and VL sequences.

Suitable assays for evaluating trogocytosis are known in the art and are e.g. described in WO 2020/012036 A1 (Genmab A/S) and WO 2020/012038 A1 (Genmab A/S)

In one embodiment, the antibody when used according to any aspect or embodiment disclosed herein, induces a higher level of trogocytosis of a CD38-expressing target cells than a reference antibody, wherein the reference antibody comprises the VH and VL region sequences of antibody C, i.e., SEQ ID NO:1 and SEQ ID NO:5, respectively, and CH and CL region sequences identical to the antibody of the invention except for the one or more mutations in E430, E345 and/or S440. In a preferred embodiment, the antibody of the invention comprises a mutation at position E430 (in a human IgG1 heavy chain), preferably E340G, and said reference antibody does not comprise said mutation at position E430. (i.e. is wt at said position), preferabley comprises a wt CH3/Fc region (in a human IgG1 heavy chain).

In some embodiments, the antibody when used according to any aspect or embodiment disclosed herein, induces a higher level of trogocytosis of CD38-expressing target cells than a reference antibody, wherein the reference antibody comprises the VH and VL region sequences of antibody B, i.e., SEQ ID NO:8 and SEQ ID NO:9, respectively and CH and CL region sequences identical to the antibody of the invention.

In one embodiment, the antibody when used according to any aspect or embodiment disclosed herein, induces a higher level of trogocytosis of CD38-expressing target cells than a reference antibody, wherein the reference antibody comprises the VH and VL region sequences of antibody B, i.e., SEQ ID NO:8 and SEQ ID NO:9, respectively, and the CH and CL region sequences of SEQ ID NO:20 (IgGm(f)) and SEQ ID NO:37 (kappa), respectively, or wherein the reference antibody is antibody B. In a preferred embodiment, the antibody of the invention comprises a mutation at position E430 (in a human IgG1 heavy chain), preferably E340G.

In one embodiment, the antibody when used according to any aspect or embodiment disclosed herein, induces trogocytosis-mediated reduction of CD38 on CD38-expressing tumor cells in said subject. In one embodiment, the antibody when used according to any aspect or embodiment disclosed herein, induces trogocytosis-mediated reduction of CD38 on CD38-expressing immune cells in said subject.

In one embodiment, said CD38-expressing immune cells are cells are CD38-expressing immunosuppressive cells In one embodiment, the trogocytosis-mediated reduction of CD38 on the CD38-expressing immunosuppressive cells reduces their immunosuppressive activity.

In one embodiment, said CD38-expressing immunosuppressive cells comprise regulatory T cells (Tregs), regulatory B cells (Bregs), myeloid-derived suppressor cells (MDSCs), immunosuppressive NK cells, immunosuppressive NKT cells, immunosuppressive antigen-expressing cells (APCs), immunosuppressive macrophages, or any combination of two or more thereof

In one embodiment, the immune cells are Tregs, thus the antibody induce trogocytosis-mediated reduction of CD38 on CD38 expressing Tregs.

### Antigen-Binding Region and Variable Regions

In the methods and uses according to the invention, the antigen-binding region of the antibody comprises one or more antibody variable domains allowing for specific binding to CD38, such as a VH region and a VL region. Similarly, the heavy and light chains comprise a VH and VL region, respectively. In the following reference to sequences in the antigen-binding region may similarly apply to sequences of the heavy and/or light chain of a antibody according to the present invention. Advantageously, the CDRs, VH region and/or VL region are similar or identical to those of antibody C, as set forth in Table 4.

In one preferred embodiment, the antigen-binding region, and/or the heavy and/or light chains comprise the CDRs of antibody C, set forth as SEQ ID NO:2 (VH-3003-C_CDR1), SEQ ID NO:3 (VH-3003-C_CDR2), SEQ ID NO:4 (VH-3003-C_CDR3), SEQ ID NO:6 (VL-3003-C_CDR1), AAS (VL-3003-C_CDR2) and SEQ ID NO:7 (VL-3003-C_CDR3). In another preferred embodiment, the VH and VL sequences are those of antibody C, i.e., the VH region comprises the sequence of SEQ ID NO:1 (VH-3003-C) and the VL region comprises the sequence of SEQ ID NO:5 (VL-3003-C).

However, it is well known in the art that mutations in the VH and VL of an antibody can be made to, for example, increase the affinity of an antibody to its target antigen, reduce its potential immunogenicity and/or to increase the yield of antibodies expressed by a host cell. Accordingly, in some embodiments, antibodies comprising variants of the CDR, VH and/or VL sequences of antibody C are also contemplated, particularly functional variants of the VL and/or VH region of antibody C. Functional variants may differ in one or more amino acids as compared to the parent VH and/or VL sequence, e.g., in one or more CDRs, but still allows the antigen-binding region to retain at least a substantial proportion (at least about 50 percent, 60 percent, 70 percent, 80 percent, 90 percent, 95 percent or more) of the affinity and/or specificity of the parent antibody. Typically, such functional variants retain significant sequence identity to the parent sequence. Exemplary variants include those which differ from the respective parent VH or VL region by 12 or less, such as 11, 10, 9, 8, 7, 6, 5, 4, 3, 2 or 1 mutation(s) such as substitutions, insertions or deletions of amino acid residues. Exemplary variants include those which differ from the VH and/or VL and/or CDR regions of the parent sequences mainly by conservative amino acid substitutions; for instance, 12, such as 11, 10, 9, 8, 7, 6, 5, 4, 3, 2 or 1 of the amino acid substitutions in the variant can be conservative. In some cases, an antibody comprising variants of the VH and/or VL of antibody C may be associated with greater affinity and/or specificity than the parent antibody. For the purpose of the present invention, VH and/or VL variants which allow for a retained or improved affinity and specificity of the antibody in its binding to CD38 are particularly preferred.

For example, WO 2011/154453 A1 discloses CD38 antibodies comprising suitable variant CDR, VH and VL region amino acid sequences, where the amino acid residues at certain positions differ from those in the CDRs, VH and VL of antibody C as shown in Table 4. These positions thus represent candidate positions where mutations in the CDR, VH and VL sequences can be made while retaining or improving affinity and specificity of the antibody in its binding to CD38. In particular, positions in the VH and VL CDRs that can be mutated in functional variants of the VH and VL of antibody C are indicated in SEQ ID NOS:40 to 43.

So, in some embodiments, one or more specific mutations are made in the CDRs as set forth in SEQ ID NOS:40 to 43, i.e., any functional variants of the VH and/or VL region comprises mutations in the CDRs as set forth in one or more of SEQ ID NO:40 (VH CDR1), SEQ ID NO:41 (VH CDR2), SEQ ID NO:42 (VH CDR3), and SEQ ID NO:44 (VL CDR3). The VH and VL regions of such an antibody variant may optionally maintain the original framework regions of antibody C. In one specific embodiment, the antigen-binding region comprises the CDRs as set forth in SEQ ID NO:40 wherein X₁ is S (VH CDR1), SEQ ID NO:41 wherein X₁ is R, X₂ is K, X₃ is A (VH CDR2), SEQ ID NO:42 wherein X₁ is A, X₂ is D and X₃ is V (VH CDR3), SEQ ID NO:43 (VL CDR1), AAS (VL CDR2) and SEQ ID NO:44 wherein X₁ is S (VL CDR3). In one specific embodiment, the antigen-binding region comprises the CDRs as set forth in SEQ ID NO:40 wherein X₁ is R (VH CDR1), SEQ ID NO:41 wherein X₁ is V, X₂ is K, X₃ is T (VH CDR2), SEQ ID NO:42 wherein X₁ is T, X₂ is A and X₃ is F (VH CDR3), SEQ ID NO:43 (VL CDR1), AAS (VL CDR2) and SEQ ID NO:44 wherein X₁ is N (VL CDR3). In one specific embodiment, the antigen-binding region comprises the CDRs as set forth in SEQ ID NO:40 wherein X₁ is S (VH CDR1), SEQ ID NO:41 wherein X₁ is R, X₂ is K, X₃ is T (VH CDR2), SEQ ID NO:42 wherein X₁ is A, X₂ is D and X₃ is V (VH CDR3), SEQ ID NO:43 (VL CDR1), AAS (VL CDR2) and SEQ ID NO:44 wherein X₁ is S (VL CDR3). In one specific embodiment, the antigen-binding region comprises the CDRs as set forth in SEQ ID NO:40 wherein X₁ is R (VH CDR1), SEQ ID NO:41 wherein X₁ is V, X₂ is K, X₃ is V (VH CDR2), SEQ ID NO:42 wherein X₁ is T, X₂ is A and X₃ is F (VH CDR3), SEQ ID NO:43 (VL CDR1), AAS (VL CDR2) and SEQ ID NO:44 wherein X₁ is N (VL CDR3).

In some embodiments, no mutation is made in the CDRs, i.e., any functional variants of the VH and/or VL region retains the CDR sequences set forth in SEQ ID NO:2, SEQ ID NO:3, SEQ ID NO:4 or SEQ ID NO:6, AAS, SEQ ID NO:7, respectively representing the VH CDR1-3 or VL CDR1-3 sequences of antibody C.

In one embodiment, the VH region comprises SEQ ID NO:1 or an amino acid sequence having at least 80% identity, such as 90%, or 95%, or 97%, or 98%, or 99%, to SEQ ID NO:1. For example, the VH may differ from SEQ ID NO:1 by 12 or less, such as 11, 10, 9, 8, 7, 6, 5, 4, 3, 2 or 1 mutations such as substitutions, insertions or deletions of amino acid residues. In one embodiment, the VH region differs from SEQ ID NO:1 only in 12 or less, such as 5 or less, such as 5, 4, 3, 2 or 1 amino acid substitutions. The amino acid substitutions may, for example, be conservative amino acid substitutions as described elsewhere herein. In a particular embodiment, no mutation is made in the VH CDRs, i.e., any variant VH retains the C CDR sequences set forth in SEQ ID NO:2, SEQ ID NO:3, SEQ ID NO:4.

In one embodiment, the VL region comprises SEQ ID NO:5 or an amino acid sequence having at least 80% identity, such as 90%, or 95%, or 97%, or 98%, or 99%, to SEQ ID NO:5. For example, the VL may differ from SEQ ID NO:5 by 12 or less, such as 11, 10, 9, 8, 7, 6, 5, 4, 3, 2 or 1 mutations such as substitutions, insertions or deletions of amino acid residues.

In one embodiment, the VL region differs from SEQ ID NO:5 only in 12 or less, such as 5 or less, such as 5, 4, 3, 2 or 1 amino acid substitutions. The amino acid substitutions may, for example, be conservative amino acid substitutions as described elsewhere herein. In a particular embodiment, no mutation is made in the VL CDRs, i.e., any variant VH retains the C CDR sequences set forth in SEQ ID NO:6, AAS, SEQ ID NO:7.

In one embodiment, the antibody comprises a VH region comprising the sequence of SEQ ID NO:1 and a VL region comprising the sequence of SEQ ID NO:5.

### Fc Region, and CH Region

Mutations in amino acid residues at positions corresponding to E430, E345 and S440 in a human IgG1 heavy chain, wherein the amino acid residues are numbered according to the EU index, can improve the ability of an antibody to induce CDC (see, e.g., Example 3). Without being bound by theory, it is believed that by substituting one or more amino acid(s) in these positions, oligomerization of the antibody can be stimulated, thereby modulating effector functions so as to, e.g., increase C1q binding, complement activation, CDC, ADCP, internalization or other relevant function(s) that may provide in vivo efficacy.

The methods and uses of the present invention relate to an antibody comprising an antigen-binding region and a Fc region comprising any of the above mutations.

In certain embodiments, the antibody binding to human CD38 according to any of the methods and uses as described herein, comprises
(a) a heavy chain comprising a VH region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4 and a human IgG1 CH region with a mutation in one or more of E430, E345 and S440, the amino acid residues being numbered according to the EU index; and
(b) a light chain comprising a VL region comprising a VL CDR1 having the sequence as set forth in SEQ ID NO:6, a VL CDR2 having the sequence AAS, and a VL CDR3 having the sequence as set forth in SEQ ID NO:7.

In other certain embodiments, the antibody binding to human CD38 according to any of the methods and uses as described herein, comprises
(a) a heavy chain comprising a VH region comprising SEQ ID NO:1 and a human IgG1 CH region with a mutation in one or more of E430, E345 and S440, the amino acid residues being numbered according to the EU index; and
(b) a light chain comprising a VL region comprising SEQ ID NO:5.

An antibody of the present invention comprises an Fc region or a human IgG1 CH region comprising a mutation in one or more of E430, E345 and S440. In the following reference to the mutations in the Fc region may similarly apply to the mutation(s) in the human IgG1 CH region.

As described herein, the position of an amino acid to be mutated in the Fc region can be given in relation to (i.e., “corresponding to”) its position in a naturally occurring (wild-type) human IgG1 heavy chain, when numbered according to the EU index. So, if the parent Fc region already contains one or more mutations and/or if the parent Fc region is, for example, an IgG2, IgG3 or IgG4 Fc region, the position of the amino acid corresponding to an amino acid residue such as, e.g., E430 in a human IgG1 heavy chain numbered according to the EU index can be determined by alignment. Specifically, the parent Fc region is aligned with a wild-type human IgG1 heavy chain sequence so as to identify the residue in the position corresponding to E430 in the human IgG1 heavy chain sequence. Any wild-type human IgG1 constant region amino acid sequence can be useful for this purpose, including any one of the different human IgG1 allotypes set forth in Table 4. This is illustrated in FIG. 1, which shows an alignment between two different human IgG1 allotypes—IgG1m(f) and IgG1 m(a)—and wild-type human IgG2, IgG3 and IgG4, specifically of the segments corresponding to residues P247 to K447 in a human IgG1 heavy chain, wherein the amino acid residues are numbered according to the EU index.

Accordingly, in the remaining paragraphs of this section and elsewhere herein, unless otherwise specified or contradicted by context, the amino acid positions referred to are those corresponding to amino acid residues in a wild-type human IgG heavy chain, wherein the amino acid residues are numbered according to the EU index:

In separate and specific embodiments, the Fc region and/or the human IgG1 CH region of the present invention comprises a mutation in only one of E430, E345 and S440; in both E430 and E345; in both E430 and S440; in both E345 and S440; or in all of E430, E345 and S440. In some embodiments, the Fc region and/or the human IgG1 CH region of the present invention comprises a mutation in only one of E430, E345 and S440; in both E430 and E345; in both E430 and S440; in both E345 and S440; or in all of E430, E345 and S440, with the proviso that any mutation in S440 is S440W or S440Y. In other separate and specific embodiments, the mutation is an amino acid substitution. In one embodiment the mutation is an amino acid substitution in only one of E430X, E345X and S440X; in both E430X and E345X; in both E430X and S440X; in both E345X and S440X; or in all of E430X, E345X and S440X, preferably with the proviso that any mutation in S440X is S440Y or S440W. More preferably, the E430X, E345X and S440X mutations are separately selected from E430G, E345K, E430S, E430F, E430T, E345Q, E345R, E345Y, S440Y and S440W.

In one embodiment, the mutation in the one or more amino acid residues is selected from the group consisting of E430G, E345K, E430S, E430F, E430T, E345Q, E345R, E345Y, S440Y and S440W.

In a preferred embodiment, the mutation in the one or more amino acid residues is selected from the group corresponding to E430G, E345K, E430S and E345Q.

In one embodiment, the mutation is in an amino acid residue corresponding to E430, such as an amino acid substitution, E430X, e.g., selected from those corresponding to E430G, E430S, E430F, or E430T. In one preferred embodiment, the mutation in the one or more amino acid residues comprises E430G. In another preferred embodiment, the mutation in the one or more amino acid residues comprises E430S, optionally wherein no mutations are made in the amino acid residues corresponding to E345 and S440.

In a particularly preferred embodiment, the mutation in the one or more amino acid residue consists of E430G, i.e., no mutations are made in the amino acid residues corresponding to E345 and S440.

In one embodiment, the mutation is in an amino acid residue corresponding to E345, such as an amino acid substitution, E345X, e.g., selected from those corresponding to E345K, E345Q, E345R and E345Y. In one preferred embodiment, the mutation in the one or more amino acid residues comprises E345K. In another preferred embodiment, the mutation in the one or more amino acid residues comprises E345Q, optionally wherein no mutations are made in the amino acid residues corresponding to E430 and S440. In a particularly preferred embodiment, the mutation in the one or more amino acid residue consists of E345K, i.e., no mutations are made in the amino acid residues corresponding to E430 and S440.

In one embodiment, the mutation is in an amino acid residue corresponding to S440, such as an amino acid substitution, S440X, typically selected from those corresponding to S440Y and S440W. In one preferred embodiment, the mutation in the one or more amino acid residues comprises S440W, optionally wherein no mutations are made in the amino acid residues corresponding to E430 and E345. In one preferred embodiment, the mutation in the one or more amino acid residues comprises S440Y, optionally wherein no mutations are made in the amino acid residues corresponding to E430 and E345.

Preferably, the antibody comprises an Fc region according to any one of the preceding sections, which Fc region is a variant of a human IgG Fc region selected from the group consisting of a human IgG1, IgG2, IgG3 and IgG4 Fc region. That is, the mutation in one or more amino acid residues corresponding to E430, E345 and S440 is/are made in a parent Fc region which is a human IgG Fc region selected from the group consisting of an IgG1, IgG2, IgG3 and IgG4 Fc region. Preferably, the parent Fc region is a naturally occurring (wild-type) human IgG Fc region, such as a human wild-type IgG1, IgG2, IgG3 or IgG4 Fc region, or a mixed isotype thereof. Thus, the Fc region of the present invention may, except for the recited mutation (in the one or more amino acid residues selected from the group corresponding to E430, E345 and S440), be a human IgG1, IgG2, IgG3 or IgG4 isotype, or a mixed isotype thereof.

In one embodiment, the parent Fc region and/or human IgG1 CH region is a wild-type human IgG1 isotype.

Thus, the Fc region of the present invention may except for the recited mutation (in the one or more amino acid residues selected from the group corresponding to E430, E345 and S440), be a human IgG1 Fc region.

In a specific embodiment, the parent Fc region and/or human IgG1 CH region is a human wild-type IgG1 m(f) isotype.

In a specific embodiment, the parent Fc region and/or human IgG1 CH region is a human wild-type IgG1m(z) isotype.

In a specific embodiment, the parent Fc region and/or human IgG1 CH region is a human wild-type IgG1 m(a) isotype.

In a specific embodiment, the parent Fc region and/or human IgG1 CH region is a human wild-type IgG1m(x) isotype.

In a specific embodiment, the parent Fc region and/or human IgG1 CH region is a human wild-type IgG1 of a mixed allotype, such as IgG1 m(za), IgG1 m(zax), IgG1m(fa), or the like.

Thus, the Fc region and/or human IgG1 CH region of the present invention may, except for the recited mutation (in the one or more amino acid residues selected from the group corresponding to E430, E345 and S440), be a human IgG1 m(f), IgG1m(a), IgG1m(x), IgG1m(z) allotype or a mixed allotype of any two or more thereof.

In a specific embodiment, the parent Fc region and/or human IgG1 CH region is a human wild-type IgG1m(za) isotype.

In a specific embodiment, the parent Fc region is a human wild-type IgG2 isotype.

In a specific embodiment, the parent Fc region is a human wild-type IgG3 isotype.

In a specific embodiment, the parent Fc region is a human wild-type IgG4 isotype.

CH region amino acid sequences of specific examples of wild-type human IgG isotypes and IgG1 allotypes are set forth in Table 4. In some embodiments, the parent Fc region comprises the CH2-CH3 or, optionally, the hinge-CH2-CH3 segments of such wild-type CH region amino acid sequences.

So, in a specific embodiment, the parent Fc region is a human wild-type IgG1 isotype comprising the amino acid residues corresponding to 231-447 in a human IgG1 heavy chain according to the EU numbering. For example, the parent Fc region may comprise amino acid residues 114 to 330 (direct numbering) of a sequence selected from the group consisting of SEQ ID NO:19, SEQ ID NO:20, SEQ ID NO:21, SEQ ID NO:22 and SEQ ID NO:23 In a specific embodiment, the parent Fc region is a human wild-type IgG1 isotype comprising the amino acid residues corresponding to 216-447 in a human IgG1 heavy chain according to the EU numbering. For example, the parent Fc region may comprise amino acid residues 99 to 330 (direct numbering) of a sequence selected from the group consisting of SEQ ID NO:19, SEQ ID NO:20, SEQ ID NO:21, SEQ ID NO:22 and SEQ ID NO:23. As described elsewhere herein for production of therapeutic antibodies, the C-terminal amino acid K447 may sometimes be deleted or removed. Hence the parent Fc region may comprise amino acid residues 114 to 329 (direct numbering) or amino acid residues 99 to 329 (direct numbering) of SEQ ID NO: 45.

In a specific embodiment, the Fc region of the present invention is a variant of a human wild-type IgG1 isotype comprising the amino acid residues corresponding to 231-447 in a human IgG1 heavy chain according to the EU numbering. For example, the Fc region may comprise amino acid residues 114 to 330 (direct numbering) of a sequence selected from the group consisting of SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32 and SEQ ID NO:33. In another embodiment the Fc region may comprise amino acid residues 114 to 329 (direct numbering) of SEQ ID NO: 46.

In a specific embodiment, the Fc region of the present invention is a variant of a human wild-type IgG1 isotype comprising the amino acid residues corresponding to 216-447 in a human IgG1 heavy chain according to the EU numbering. For example, the Fc region may comprise amino acid residues 99 to 330 (direct numbering) of a sequence selected from the group consisting of SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32 and SEQ ID NO:33. In another embodiment the Fc region may comprise amino acid residues 99 to 329 (direct numbering) of SEQ ID NO: 46.

So, the present invention can be applied to antibody molecules having a human IgG1 heavy chain, such as a human IgG1 heavy chain comprising a human IgG1 CH region amino acid sequence comprising SEQ ID NO:19 (IgGm(za). Thus, the human IgG1 CH region may comprise, except for the recited mutation, the sequence of SEQ ID NO:19.

The present invention can also be applied to antibody molecules having a human IgG1 heavy chain, such as a human IgG1 heavy chain comprising a human IgG1 CH region amino acid sequence comprising SEQ ID NO:20 (IgGm(f)) or SEQ ID NO: 45. Thus, the human IgG1 CH region may comprise, except for the recited mutation, the sequence of SEQ ID NO:20.

In another embodiment the human IgG1 CH region may comprise, except for the recited mutation, the sequence of SEQ ID NO: 45.

The present invention can also be applied to antibody molecules having a human IgG1 heavy chain, such as a human IgG1 heavy chain comprising a human IgG1 CH region amino acid sequence comprising SEQ ID NO:21 (IgGm(z)). Thus, the human IgG1 CH region may comprise, except for the recited mutation, the sequence of SEQ ID NO:21.

The present invention can also be applied to antibody molecules having a human IgG1 heavy chain, such as a human IgG1 heavy chain comprising a human IgG1 CH region amino acid sequence comprising, SEQ ID NO:22 (IgGm(a)). Thus, the human IgG1 CH region may comprise, except for the recited mutation, the sequence of SEQ ID NO:22.

The present invention can also be applied to antibody molecules having a human IgG1 heavy chain, such as a human IgG1 heavy chain comprising a human IgG1 CH region amino acid sequence comprising SEQ ID NO:23 (IgG1 m(x)). Thus, the human IgG1 CH region may comprise, except for the recited mutation, the sequence of SEQ ID NO:23.

In other separate and specific embodiments, the human IgG1 CH region comprises an amino acid sequence selected from the group consisting of SEQ ID NO:24 to SEQ ID NO:33 and SEQ ID NO: 45.

In a specific embodiment, the human IgG1 CH region comprises SEQ ID NO:24 (IgG1m(f)-E430G) or SEQ ID NO:46, optionally wherein the light chain comprises a CL comprising SEQ ID NO:37.

In a specific embodiment, the antibody of the present invention is a monospecific antibody comprising two HCs that are identical in amino acid sequence and two LCs that are identical in amino acid sequence.

The present invention can also be applied to antibody molecules having a human IgG2 heavy chain, such as a human IgG2 heavy chain comprising a human IgG2 CH region amino acid sequence comprising SEQ ID NO:34.

The present invention can also be applied to antibody molecules having a human IgG3 heavy chain, such as a human IgG3 heavy chain comprising a human IgG3 CH region amino acid sequence comprising SEQ ID NO:35.

The present invention can also be applied to antibody molecules having a human IgG4 heavy chain, such as a human IgG4 heavy chain comprising a human IgG4 CH region amino acid sequence comprising SEQ ID NO:36.

However, Fc regions comprising one or more further mutations, i.e., mutations in one or more other amino acid residues other than those corresponding to E430, E345 and S440 in a human IgG1 heavy chain when numbered according to the EU index, are also contemplated for the antibodies disclosed herein. Also or alternatively, the Fc region may be a mixed isotype, e.g., where different CH regions derive from different IgG isotypes. Accordingly, as described in more detail below, the parent Fc region may already comprise one or more further mutations as compared to such a wild-type (naturally occurring) human IgG Fc region, or may be a mixed isotype.

In one embodiment, the parent Fc region into which a mutation in one or more amino acid residues selected from the group corresponding to E430, E345 and S440 is introduced, is a human IgG Fc region which comprises one or more further mutations as compared to a wild-type human IgG1, IgG2, IgG3 and IgG4 Fc region, e.g., as set forth in one of SEQ ID NO:19, SEQ ID NO:20, SEQ ID NO:21, SEQ ID NO:22, SEQ ID NO:23, SEQ ID NO:34, SEQ ID NO:35 and SEQ ID NO:36. Expressed in an alternative manner, the Fc region comprising a mutation in E430, E345 and/or S440 may differ also in one or more further mutations from a reference Fc region, such as a reference wild-type human IgG1, IgG2, IgG3 and IgG4 Fc region, e.g., as set forth in one of SEQ ID NO:19, SEQ ID NO:20, SEQ ID NO:21, SEQ ID NO:22, SEQ ID NO:23, SEQ ID NO:34, SEQ ID NO:35 and SEQ ID NO:36. For example, except for the mutation in one or more amino acid residues selected from the group corresponding to E430, E345 and S440, the Fc region may differ from the wild-type Fc region by 12 or less, such as 11, 10, 9, 8, 7, 6, 5, 4, 3, 2 or 1 mutations such as substitutions, insertions or deletions of amino acid residues. For example the C-terminal amino acid Lys (K) at position 447 (Eu numbering) may have been deleted. Some host cells which are used for production of an antibody may contain enzymes capable of removing the Lys at position 447, and such removal may not be homogenous. Therapeutic antibodies may therefore be produced without the C-terminal Lys (K) to increase the homogenicity of the product. Methods for producing antibodies without the C-terminal Lys (K) are well-known to a person skilled in the art and include genetic engineering of the nucleic acid expressing said antibody, enzymatic methods and use of specific host cells. Thus, for example the parent Fc region may comprise the sequence as set forth in SEQ ID NO: 45.

Preferably, any such one or more further mutations do not reduce the ability of the antibody as disclosed herein, i.e., an antibody comprising a mutation in one or more amino acid residues selected from the group corresponding to E430, E345 and S440 in a human IgG1 heavy chain, to induce CDC and/or ADCC. More preferably, any such one or more further mutations do not reduce the ability of the antibody to induce CDC. Most preferably, any such one or more further mutations do not reduce the ability of the antibody to induce either one of CDC and ADCC. Candidates for the one or more further mutations can, for example, be tested in CDC or ADCC assays, e.g., as disclosed herein, such as in Examples 3 and 4. For example, the CDC of an antibody as described herein, e.g., IgG1-C-E430G, can be tested in the assay of Example 3 or an assay as described in the next section (or a similar assay) with and without specific candidates for one or more further mutations, so as to ascertain the effect of the candidate further mutation(s) on the ability of the antibody to induce CDC. Likewise, the ADCC of an antibody as described herein, e.g., IgG1-C-E430G, can be tested in the assay of Example 4 or an assay as described in the next section (or a similar assay) with and without a specific candidate for a further mutation so as to ascertain the effect of the candidate further mutation on the ability on the antibody to induce ADCC.

Preferably, in an antibody of the present invention comprising two HCs and two LCs, the Fc regions in the first and second HC are identical such that the Fc region, in dimerized form, is a homodimer.

However, in some embodiments, in an antibody comprising two HCs and two LCs, the Fc region in the first HC may differ in one or more amino acids from the Fc region in the second HC, such that the Fc region, in dimerized form, is a heterodimer. For example, the mutation in one or more amino acid residues selected from the group corresponding to E430, E345 and S440 in an IgG1 heavy chain, wherein the amino acid residues are numbered according to the EU index, may only be present in one of the Fc regions. Accordingly, in some embodiments, one Fc region may be SEQ ID NO:45 or a human wild-type IgG Fc region selected from SEQ ID NO:19, SEQ ID NO:20, SEQ ID NO:21, SEQ ID NO:22, SEQ ID NO:23, SEQ ID NO:34, SEQ ID NO:35 and SEQ ID NO:36 while the other Fc region may be identical except for a mutation in said one or more amino acid residues selected from the group corresponding to E430, E345 and S440 in an IgG1 heavy chain.

In one embodiment, the antibody according to any aspect or embodiment herein is, except for the recited mutations, a human antibody.

In one embodiment, the antibody according to any aspect or embodiment herein is, except for the recited mutations, a full-length antibody, such as a human full-length antibody.

In one embodiment, the antibody according to any aspect or embodiment herein is, except for the recited mutations, a bivalent antibody, such as a human bivalent antibody, such as a human bivalent full-length antibody.

In one embodiment, the antibody according to any aspect or embodiment herein is, except for the recited mutations, a monoclonal antibody, such as a human monoclonal antibody, such as a human bivalent monoclonal antibody, such as a human bivalent full-length monoclonal antibody.

In a preferred embodiment, the antibody according to any aspect or embodiment herein is, except for the recited mutations, an IgG1 antibody, such as a full length IgG1 antibody, such as a human full-length IgG1 antibody, optionally a human monoclonal full-length bivalent IgG1,κ antibody, e.g. a human monoclonal full-length bivalent IgG1m(f),κ antibody.

An antibody according to the present invention is advantageously in a bivalent monospecific format, comprising two antigen-binding regions binding to the same epitope. However, bispecific formats where one of the antigen-binding regions binds to a different epitope are also contemplated. So, the antibody according to any aspect or embodiment herein can, unless contradicted by context, be either a monospecific antibody or a bispecific antibody.

So, in one embodiment, the antibody according to any aspect or embodiment herein is, except for the recited mutations, a monospecific antibody, such as a human monospecific antibody, such as a human full-length monospecific antibody, such as a human full-length monospecific bivalent monoclonal antibody, such as a human full-length bivalent monospecific monoclonal antibody.

In another embodiment, the antibody according to any aspect or embodiment herein is, except for the recited mutations, a bispecific antibody, such as a full-length bispecific antibody, optionally a full-length bispecific and bivalent IgG1,κ antibody.

### Formulations

In one aspect, the antibody of the invention is comprised in a pharmaceutical composition as described in WO 2021/144457 A1.

Thus, in one aspect, the antibody of the invention is comprised in a pharmaceutical composition, said pharmaceutical composition comprising, consisting or consisting essentially of
(a) the antibody
(b) 5-40 mM histidine or acetate;
(c) 100-400 mM sorbitol or sucrose; and
(d) a surfactant.

In one aspect, the antibody of the invention is comprised in a pharmaceutical composition, said pharmaceutical composition consisting of
a) the antibody
b) 5-40 mM histidine or acetate;
c) 100-400 mM sorbitol or sucrose; and
d) a surfactant,
   in aqueous solution.

In some embodiments, in the pharmaceutical composition, a) may be from 1 to 80 mg/mL, such as 1 to 60 mg/ml, 1 to 40 mg/mL, 1 to 30 mg/ml or 1 to 25 mg/ml; 2 to 80 mg/mL, such as 2 to 40 mg/mL or 2 to 30 mg/ml; or 10 to 80 mg/mL, such as 10 to 40 mg/mL or 10 to 30 mg/ml; or 15 to 80 mg/ml, such as 15 to 40 mg/ml, such as 15 to 25 mg/ml, such as 2 mg/ml, 4 mg/mL, 6 mg/mL, 8 mg/mL, 10 mg/mL, 12 mg/mL, 14 mg/mL, 16 mg/mL, 18 mg/mL, 20 mg/mL, 22 mg/mL, 24 mg/mL, 26 mg/mL, 28 mg/mL, 30 mg/mL, 32 mg/mL, 34 mg/mL, 36 mg/mL, 38 mg/mL, 40 mg/mL, or 50 mg/mL of the antibody. In one embodiment, a) is about 20 mg/mL of the antibody. In a particularly contemplated embodiment, a) is 20 mg/mL of the antibody.

In some embodiments, in the pharmaceutical composition b) may be from 5 to 30 mM, such as 5 to 25 mM, such as 10 mM, 11, mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM or 30 mM of histidine or acetate. In one embodiment, b) is about 20 mM, such as 20 mM, of histidine or acetate. In one specific embodiment, b) is acetate. In a particularly contemplated embodiment, b) is histidine.

In some embodiments, in the pharmaceutical composition, c) may be from 100 to 350 mM, such as 100 to 300 mM, 100 to 260 mM, 100 to 200 mM, 150 to 350 mM, 200 to 300 mM, 200 to 260 mM, 200 to 350 mM, 200 to 300 mM, 200 to 260 mM, 230 to 350 mM, 230 to 300 mM, 230 to 260 mM or 240 to 260 mM; such as 245 mM, 246 mM, 247 mM, 248 mM, 249 mM, 250 mM, 251 mM, 252 mM, 253 mM, 254 mM, or 255 mM of sorbitol or sucrose. In one embodiment, c) is about 250 mM, such as 250 mM, of sorbitol or sucrose. In one embodiment, c) is sucrose. In a particularly contemplated embodiment, c) is sorbitol.

The pharmaceutical composition may, for example, have a pH from 5.0 to 6.5, such as 5.5 to 6.5, such as 5.6 to 6.5, 5.7 to 6.5, 5.8 to 6.5, 5.9 to 6.5, 6.0 to 6.5, 5.5 to 6.4, 5.5 to 6.3, 5.5 to 6.2, 5.5 to 6.1, 5.5 to 6.0, 5.7 to 6.3, 5.8 to 6.2, 5.9 to 6.1. In one embodiment, the pH is about 6. In one embodiment, the pH is 6, such as 6.0.

Surfactants suitable for the pharmaceutical composition are known in the art and may, for example, be selected from the group comprising glycerol monooleate, benzethonium chloride, sodium docusate, phospholipids, polyethylene alkyl ethers, sodium lauryl sulfate and tricaprylin, benzalkonium chloride, citrimide, cetylpyridinium chloride and phospholipids, alpha tocopherol, glycerol monooleate, myristyl alcohol, phospholipids, poloxamers, polyoxyethylene alkyl ethers, polyoxyethylene castor oil derivatives, polyoxyethylene sorbintan fatty acid esters, polyoxyethylene sterarates, polyoxyl hydroxystearate, polyoxylglycerides, polysorbates, propylene glycol dilaurate, propylene glycol monolaurate, sorbitan esters sucrose palmitate, sucrose stearate, tricaprylin and TPGS. In one particular embodiment, the surfactant is a polysorbate. Preferably, the surfactant is polysorbate 20 or 80. In one embodiment, the surfactant is polysorbate 20 (PS20). In a particularly contemplated embodiment, the surfactant is polysorbate 80 (PS80).

The concentration of the surfactant is typically from about 0.005% to 0.5% w/v, such as from about 0.01 to 0.1% w/v, such as from about 0.01 to 0.09% w/v such as from about 0.01 to 0.06% w/v such as from about 0.01 to 0.05% w/v such as 0.02% w/v or 0.03% w/v or 0.04% w/v or 0.05% w/v, or 0.06% w/v. In one embodiment, the concentration of the surfactant is about 0.04% w/v, such as 0.04% w/v.

In a one embodiment, the pharmaceutical composition has a pH of 5.9 to 6.1, such as about 6 or 6.0, and comprises or consists essentially of:
a) 1 to 80 mg/mL of the antibody
b) 15 to 40 mM histidine
c) 200 to 300 mM sorbitol
d) 0.01% to 0.1% w/v of a surfactant.

In one embodiment, the pharmaceutical composition has a pH of 5.9 to 6.1, such as about 6 or 6.0 and comprises or consists essentially of:
a) 10 to 40 mg/mL of the antibody
b) 15 to 40 mM histidine
c) 200 to 300 mM sorbitol
d) 0.02% to 0.06% w/v of a surfactant.

In one embodiment, the pharmaceutical composition has a pH of 5.9 to 6.1, such as about 6 or 6.0 and comprises or consists essentially of:
a) 10 to 40 mg/mL of the antibody
b) 15 to 25 mM histidine
c) 240 to 260 mM sorbitol
d) 0.02% to 0.06% w/v of a surfactant.

In a particular embodiment, the surfactant in d) is polysorbate, such as polysorbate 20 or polysorbate 80. In one embodiment, the surfactant is polysorbate 20. In one particularly contemplated embodiment, the surfactant is polysorbate 80.

One particular example, the pharmaceutical composition has a pH of about 6 and comprises or consists essentially of
a) about 20 mg/mL of the antibody,
b) about 20 mM histidine,
c) about 250 mM sorbitol, and
d) about 0.04% w/v of polysorbate 80.

Another particular example, the pharmaceutical composition has a pH of 6 and comprises, consists or consists essentially of
a) 20 mg/mL of the antibody,
b) 20 mM histidine,
c) 250 mM sorbitol, and
d) 0.04% w/v of polysorbate 80,
   in aqueous solution.

In some embodiments, the pharmaceutical composition according to the present invention is a concentrate to be diluted, typically prior to or in connection with administration to a subject or patient. Suitable diluents are known in the art. Preferred diluents include, without limitation, saline (0.9% NaCl) and dextrose (e.g., 5% w/v) in aqueous solution.

The pharmaceutical composition according to any aspect or embodiment herein may comprise a CD38 antibody further characterized by other or additional features, as described elsewhere herein.

Thus, in a specific embodiment, the pharmaceutical composition has a pH of about 6 and comprises or consists essentially of
a) about 20 mg/mL of an antibody binding to human CD38,
b) about 20 mM histidine,
c) about 250 mM sorbitol, and
d) about 0.04% w/v of polysorbate 80,
wherein the antibody is a full-length bivalent antibody comprising, consisting, or consisting essentially of two heavy chains and two light chains, wherein
   each heavy chain comprises a VH region and a CH region, wherein the VH region comprises SEQ ID NO:1 and the CH region comprises SEQ ID NO:24 or SEQ ID NO:46, and
   each light chain comprises a VL region and a CL region, wherein the VL region comprises SEQ ID NO:5 and the CL region comprises SEQ ID NO:37.

In one embodiment, the pharmaceutical composition has a pH of about 6 and comprises, consists or consists essentially of
a) 20 mg/mL of the antibody,
b) 20 mM histidine,
c) 250 mM sorbitol, and
d) 0.04% w/v of polysorbate 80,
   in aqueous solution.

### Uses

In one aspect, the invention relates to the anti-CD38 antibody or a (pharmaceutical) composition comprising said antibody according to any aspect or embodiment herein, for use in treating or preventing a hematological malignancy involving cells expressing CD38 as described herein, such as for use in the treatment or prevention of (relapsed or refractory) multiple myeloma, for example wherein said antibody is administered to a subject at a dose of at least (about) 4 mg/kg body weight, such as between (about) 4 mg/kg to (about) 24 mg/kg body weight or between (about) 8 mg/kg to (about) 16 mg/kg body weight, preferably at (about) 16 mg/kg body weight.

In one aspect, the invention relates to the anti-CD38 antibody or a (pharmaceutical) composition comprising said antibody according to any aspect or embodiment herein for use in treating or preventing a hematological malignancy in a subject comprising cells expressing human CD38 as described herein, such as for use in the treatment or prevention of (relapsed or refractory) multiple myeloma, for example wherein said antibody is administered to a subject at a dose of at least (about) 4 mg/kg body weight, such as between (about) 4 mg/kg to (about) 24 mg/kg body weight or between (about) 8 mg/kg to (about) 16 mg/kg body weight, preferably at (about) 16 mg/kg body weight.

In one aspect, the invention provides the anti-CD38 antibody or a (pharmaceutical) composition comprising said antibody according to any of the embodiments and aspects as described herein, for use in the prevention or treatment of a hematological malignancy as described herein, such as for use in the treatment of (relapsed or refractory) multiple myeloma, for example wherein said antibody is administered to a subject at a dose of at least (about) 4 mg/kg body weight, such as between (about) 4 mg/kg to (about) 24 mg/kg body weight or between (about) 8 mg/kg to (about) 16 mg/kg body weight, preferably at (about) 16 mg/kg body weight.

In one aspect, the invention relates to the anti-CD38 antibody or a (pharmaceutical) composition comprising said antibody according to any aspect or embodiment herein for use as a medicament for treating or preventing a hematological malignancy as described herein, such as for use as a medicament for the treatment or prevention of (relapsed or refractory) multiple myeloma, for example wherein said antibody is administered to a subject at a dose of at least (about) 4 mg/kg body weight, such as between (about) 4 mg/kg to (about) 24 mg/kg body weight or between (about) 8 mg/kg to (about) 16 mg/kg body weight, preferably at (about) 16 mg/kg body weight.

In one aspect, the invention provides the use of the anti-CD38 antibody or a (pharmaceutical) composition comprising said antibody according to any of the embodiments and aspects as described herein, for the manufacture of a medicament for the prevention or treatment of a hematological malignancy as described herein, such as for the manufacture of a medicament for the treatment or prevention of (relapsed or refractory) multiple myeloma, for example wherein said antibody is administered to a subject at a dose of at least (about) 4 mg/kg body weight, such as between (about) 4 mg/kg to (about) 24 mg/kg body weight or between (about) 8 mg/kg to (about) 16 mg/kg body weight, preferably at (about) 16 mg/kg body weight.

### Sequences

TABLE 4
Amino acid and nucleic acid sequences

| SEQ | | |
|---|---|---|
| ID | | |
| NO: | DESIGNATION | SEQUENCE |
| 1 | VH-3003-C | QVQLVQSGAEVKKPGSSVKVSCKAFGGTFSSYAISWVRQAPGQGLEWMGRII |
| | | RFLGIANYAQKFQGRVTLIADKSTNTAYMELSSLRSEDTAVYYCAGEPGERDP |
| | | DAVDIWGQGTMVTVSS |
| 2 | VH-3003-C_CDR1 | GGTFSSYA |
| 3 | VH-3003-C_CDR2 | IIRFLGIA |
| 4 | VH-3003-C_CDR3 | AGEPGERDPDAVDI |
| 5 | VL(Kappa)-3003-C | DIQMTQSPSSLSASVGDRVTITCRASQGIRSWLAWYQQKPEKAPKSLIYAASS |
| | | LQSGVPSRFSGSGSGTDFTLTISSLQPEDFATYYCQQYNSYPLTFGGGTKVEI |
| | | K |
| 6 | VL(Kappa)-3003-C_CDR1 | QGIRSW |
| | VL(Kappa)-3003-C_CDR2 | AAS |
| 7 | VL(Kappa)-3003-C_CDR3 | QQYNSYPLT |
| 8 | VH-3003-B | EVQLLESGGGLVQPGGSLRLSCAVSGFTFNSFAMSWVRQAPGKGLEWVSAI |
| | | SGSGGGTYYADSVKGRFTISRDNSKNTLYLQMNSLRAEDTAVYFCAKDKILW |
| | | FGEPVFDYWGQGTLVTVSS |
| 9 | VL(Kappa)-3003-B | EIVLTQSPATLSLSPGERATLSCRASQSVSSYLAWYQQKPGQAPRLLIYDASN |
| | | RATGIPARFSGSGSGTDFTLTISSLEPEDFAVYYCQQRSNWPPTFGQGTKVEI |
| | | K |
| 10 | VH-3003-A | QVQLVQSGAEVKKPGSSVKVSCKASGGTFSSYAFSWVRQAPGQGLEWMGR |
| | | VIPFLGIANSAQKFQGRVTITADKSTSTAYMDLSSLRSEDTAVYYCARDDIAAL |
| | | GPFDYWGQGTLVTVSS |
| 11 | VL(Kappa)-3003-A | DIQMTQSPSSLSASVGDRVTITCRASQGISSWLAWYQQKPEKAPKSLIYAASS |
| | | LQSGVPSRFSGSGSGTDFTLTISSLQPEDFATYYCQQYNSYPRTFGQGTKVEI |
| | | K |
| 12 | VH-gp120-b12 | QVQLVQSGAEVKKPGASVKVSCQASGYRFSNFVIHWVRQAPGQRFEWMGW |
| | | INPYNGNKEFSAKFQDRVTFTADTSANTAYMELRSLRSADTAVYYCARVGPY |
| | | SWDDSPQDNYYMDVWGKGTTVIVSS |
| 13 | VH-gp120-b12_CDR1 | GYRFSNFV |
| 14 | VH-gp120-b12_CDR2 | INPYNGNK |
| 15 | VH-gp120-b12_CDR3 | ARVGPYSWDDSPQDNYYMDV |
| 16 | VL-gp120-b12 | EIVLTQSPGTLSLSPGERATFSCRSSHSIRSRRVAWYQHKPGQAPRLVIHGVS |
| | | NRASGISDRFSGSGSGTDFTLTITRVEPEDFALYYCQVYGASSYTFGQGTKLE |
| | | RK |
| 17 | VL-gp120-b12_CDR1 | HSIRSRR |
| | VL-gp120-b12_CDR2 | GVS |
| 18 | VL-gp120-b12_CDR3 | QVYGASSYT |
| 19 | constant region human HC | ASTKGPSVFPLAPSSKSTSGGTAALGCLVKDYFPEPVTVSWNSGALTSGVHT |
| | IgG1m(za) (Uniprot entry | FPAVLQSSGLYSLSSVVTVPSSSLGTQTYICNVNHKPSNTKVDKKVEPKSCDK |
| | P01857) | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSDIAVE |
| | | WESNGQPENNYKTTPPVLDSDGSFFLYSKLTVDKSRWQQGNVFSCSVMHEA |
| | | LHNHYTQKSLSLSPGK |
| 20 | constant region human HC | ASTKGPSVFPLAPSSKSTSGGTAALGCLVKDYFPEPVTVSWNSGALTSGVHT |
| | IgG1m(f) | FPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEA |
| | | LHNHYTQKSLSLSPGK |
| 21 | constant region human HC | ASTKGPSVFPLAPSSKSTSGGTAALGCLVKDYFPEPVTVSWNSGALTSGVHT |
| | IgG1m(z) | FPAVLQSSGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEA |
| | | LHNHYTQKSLSLSPGK |
| 22 | constant region human HC | ASTKGPSVFPLAPSSKSTSGGTAALGCLVKDYFPEPVTVSWNSGALTSGVHT |
| | IgG1m(a) | FPAVLQSSGLYSLSSVVTVPSSSLGTQTYICNVNHKPSNTKVDKPVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSDIAVE |
| | | WESNGQPENNYKTTPPVLDSDGSFFLYSKLTVDKSRWQQGNVFSCSVMHEA |
| | | LHNHYTQKSLSLSPGK |
| 23 | constant region human HC | ASTKGPSVFPLAPSSKSTSGGTAALGCLVKDYFPEPVTVSWNSGALTSGVHT |
| | IgG1m(x) | FPAVLQSSGLYSLSSVVTVPSSSLGTQTYICNVNHKPSNTKVDKPVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEG |
| | | LHNHYTQKSLSLSPGK |
| 24 | constant region human HC | ASTKGPSVFPLAPSSKSTSGGTAALGCLVKDYFPEPVTVSWNSGALTSGVHT |
| | IgG1m(f)-E430G | FPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHGA |
| | | LHNHYTQKSLSLSPGK |
| 25 | constant region human HC | ASTKGPSVFPLAPSSKSTSGGTAALGCLVKDYFPEPVTVSWNSGALTSGVHT |
| | IgG1m(f)-E430S | FPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHSA |
| | | LHNHYTQKSLSLSPGK |
| 26 | constant region human HC | ASTKGPSVFPLAPSSKSTSGGTAALGCLVKDYFPEPVTVSWNSGALTSGVHT |
| | IgG1m(f)-E430F | FPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHFA |
| | | LHNHYTQKSLSLSPGK |
| 27 | constant region human HC | ASTKGPSVFPLAPSSKSTSGGTAALGCLVKDYFPEPVTVSWNSGALTSGVHT |
| | IgG1m(f)-E430T | FPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHTA |
| | | LHNHYTQKSLSLSPGK |
| 28 | constant region human HC | ASTKGPSVFPLAPSSKSTSGGTAALGCLVKDYFPEPVTVSWNSGALTSGVHT |
| | IgG1m(f)-E345K | FPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPRKPQVYTLPPSREEMTKNQVSLTCLVKGFYPSDIAVE |
| | | WESNGQPENNYKTTPPVLDSDGSFFLYSKLTVDKSRWQQGNVFSCSVMHEA |
| | | LHNHYTQKSLSLSPGK |
| 29 | constant region human HC | ASTKGPSVFPLAPSSKSTSGGTAALGCLVKDYFPEPVTVSWNSGALTSGVHT |
| | IgG1m(f)-E345Q | FPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPRQPQVYTLPPSREEMTKNQVSLTCLVKGFYPSDIAVE |
| | | WESNGQPENNYKTTPPVLDSDGSFFLYSKLTVDKSRWQQGNVFSCSVMHEA |
| | | LHNHYTQKSLSLSPGK |
| 30 | constant region human HC | ASTKGPSVFPLAPSSKSTSGGTAALGCLVKDYFPEPVTVSWNSGALTSGVHT |
| | IgG1m(f)-E345R | FPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPRRPQVYTLPPSREEMTKNQVSLTCLVKGFYPSDIAVE |
| | | WESNGQPENNYKTTPPVLDSDGSFFLYSKLTVDKSRWQQGNVFSCSVMHEA |
| | | LHNHYTQKSLSLSPGK |
| 31 | constant region human HC | ASTKGPSVFPLAPSSKSTSGGTAALGCLVKDYFPEPVTVSWNSGALTSGVHT |
| | IgG1m(f)-E345Y | FPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPRYPQVYTLPPSREEMTKNQVSLTCLVKGFYPSDIAVE |
| | | WESNGQPENNYKTTPPVLDSDGSFFLYSKLTVDKSRWQQGNVFSCSVMHEA |
| | | LHNHYTQKSLSLSPGK |
| 32 | constant region human HC | ASTKGPSVFPLAPSSKSTSGGTAALGCLVKDYFPEPVTVSWNSGALTSGVHT |
| | IgG1m(f)-S440W | FPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEA |
| | | LHNHYTQKWLSLSPGK |
| 33 | constant region human HC | ASTKGPSVFPLAPSSKSTSGGTAALGCLVKDYFPEPVTVSWNSGALTSGVHT |
| | IgG1m(f)-S440Y | FPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEA |
| | | LHNHYTQKYLSLSPGK |
| 34 | constant region human HC | ASTKGPSVFPLAPCSRSTSESTAALGCLVKDYFPEPVTVSWNSGALTSGVHT |
| | IgG2 (Uniprot entry | FPAVLQSSGLYSLSSVVTVPSSNFGTQTYTCNVDHKPSNTKVDKTVERKCCV |
| | P01859) | ECPPCPAPPVAGPSVFLFPPKPKDTLMISRTPEVTCVVVDVSHEDPEVQFNW |
| | | YVDGVEVHNAKTKPREEQFNSTFRVVSVLTVVHQDWLNGKEYKCKVSNKGL |
| | | PAPIEKTISKTKGQPREPQVYTLPPSREEMTKNQVSLTCLVKGFYPSDIAVEW |
| | | ESNGQPENNYKTTPPMLDSDGSFFLYSKLTVDKSRWQQGNVFSCSVMHEAL |
| | | HNHYTQKSLSLSPGK |
| 35 | constant region human HC | ASTKGPSVFPLAPCSRSTSGGTAALGCLVKDYFPEPVTVSWNSGALTSGVHT |
| | IgG3 (Uniprot entry | FPAVLQSSGLYSLSSVVTVPSSSLGTQTYTCNVNHKPSNTKVDKRVELKTPLG |
| | P01860) | DTTHTCPRCPEPKSCDTPPPCPRCPEPKSCDTPPPCPRCPEPKSCDTPPPCP |
| | | RCPAPELLGGPSVFLFPPKPKDTLMISRTPEVTCVVVDVSHEDPEVQFKWYV |
| | | DGVEVHNAKTKPREEQYNSTFRVVSVLTVLHQDWLNGKEYKCKVSNKALPAP |
| | | IEKTISKTKGQPREPQVYTLPPSREEMTKNQVSLTCLVKGFYPSDIAVEWESS |
| | | GQPENNYNTTPPMLDSDGSFFLYSKLTVDKSRWQQGNIFSCSVMHEALHNR |
| | | FTQKSLSLSPGK |
| 36 | constant region human HC | ASTKGPSVFPLAPCSRSTSESTAALGCLVKDYFPEPVTVSWNSGALTSGVHT |
| | IgG4 (Uniprot entry | FPAVLQSSGLYSLSSVVTVPSSSLGTKTYTCNVDHKPSNTKVDKRVESKYGPP |
| | P01861) | CPSCPAPEFLGGPSVFLFPPKPKDTLMISRTPEVTCVVVDVSQEDPEVQFNW |
| | | YVDGVEVHNAKTKPREEQFNSTYRVVSVLTVLHQDWLNGKEYKCKVSNKGL |
| | | PSSIEKTISKAKGQPREPQVYTLPPSQEEMTKNQVSLTCLVKGFYPSDIAVEW |
| | | ESNGQPENNYKTTPPVLDSDGSFFLYSRLTVDKSRWQEGNVFSCSVMHEAL |
| | | HNHYTQKSLSLSLGK |
| 37 | constant region human | RTVAAPSVFIFPPSDEQLKSGTASVVCLLNNFYPREAKVQWKVDNALQSGNS |
| | Kappa LC | QESVTEQDSKDSTYSLSSTLTLSKADYEKHKVYACEVTHQGLSSPVTKSFNR |
| | | GEC |
| 38 | Human CD38 (Uniprot entry | MANCEFSPVSGDKPCCRLSRRAQLCLGVSILVLILVVVLAVVVPRWRQQWSG |
| | P28907) | PGTTKRFPETVLARCVKYTEIHPEMRHVDCQSVWDAFKGAFISKHPCNITEED |
| | | YQPLMKLGTQTVPCNKILLWSRIKDLAHQFTQVQRDMFTLEDTLLGYLADDLT |
| | | WCGEFNTSKINYQSCPDWRKDCSNNPVSVFWKTVSRRFAEAACDVVHVMLN |
| | | GSRSKIFDKNSTFGSVEVHNLQPEKVQTLEAWVIHGGREDSRDLCQDPTIKEL |
| | | ESIISKRNIQFSCKNIYRPDKFLQCVKNPEDSSCTSEI |
| 39 | hisCD38 | HHHHHHRWRQTWSGPGTTKRFPETVLARCVKYTEIHPEMRHVDCQSVWDA |
| | | FKGAFISKHPCNITEEDYQPLMKLGTQTVPCNKILLWSRIKDLAHQFTQVQRD |
| | | MFTLEDTLLGYLADDLTWCGEFNTSKINYQSCPDWRKDCSNNPVSVFWKTVS |
| | | RRFAEAACDVVHVMLNGSRSKIFDKNSTFGSVEVHNLQPEKVQTLEAWVIHG |
| | | GREDSRDLCQDPTIKELESIISKRNIQFSCKNIYRPDKFLQCVKNPEDSSCTSEI |
| 40 | VH CDR1 variants | GGTFX₁SYA, wherein X₁ is S or R |
| 41 | VH CDR2 variants | IIX₁FLGX₂X₃, wherein X₁ is R or V; X₂ is I or K; and X₃ |
| | | is A, T or V, such as A or T |
| 42 | VH CDR3 variants | X₁GEPGX₂RDPDAX₃DI, wherein X₁ is A or T; X₂ is E, D or |
| | | A, such as E or D; and X₃ is V or F |
| 43 | VL CDR1 | QGIRSW |
| | VL CDR2 | AAS |
| 44 | VL CDR3 variants | QQYNX₁YPLT, wherein X₁ is S or N |
| 45 | constant region human HC | ASTKGPSVFPLAPSSKSTSGGTAALGCLVKDYFPEPVTVSWNSGALTSGVHT |
| | IgG1m(f) without Lys (K) | FPAVLQSSGLYSLSSVVTVPSSSLGTQTYICNVNHKPSNTKVDKRVEPKSCDK |
| | at position 447 according | THTCPPCPAPELLGGPSVFLFPPKPKDTLMISRTPEVTCVVVDVSHEDPEVKF |
| | to Eu numbering | NWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEA |
| | | LHNHYTQKSLSLSPG |
| 46 | constant region human HC | ASTKGPSVFPLAPSSKSTSGGTAALGCLVKDYFPEPVTVSWNSGALTSGVHT |
| | IgG1m(f)-E430G, without | FPAVLQSSGLYSLSSVVTVPSSSLGTQTYICNVNHKPSNTKVDKRVEPKSCDK |
| | Lys (K) at position 447 | THTCPPCPAPELLGGPSVFLFPPKPKDTLMISRTPEVTCVVVDVSHEDPEVKF |
| | according to Eu numbering | NWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHGA |
| | | LHNHYTQKSLSLSPG |

### LIST OF REFERENCES

Each reference in this list, or cited elsewhere herein, is hereby specifically incorporated by reference in its entirety.
Antonelli, A., P. Fallahi, et al. (2001). “Anti-CD38 autoimmunity in patients with chronic autoimmune thyroiditis or Graves' disease.” Clin Exp Immunol 126(3): 426-431.
Ausiello, C. M., F. Urbani, et al. (2000). “Functional topography of discrete domains of human CD38.” Tissue Antigens 56(6): 539-547.
Brezski, R. J. and G. Georgiou (2016). “Immunoglobulin isotype knowledge and application to Fc engineering.” Curr Opin Immunol 40: 62-69.
Chatterjee, S., A. Daenthanasanmak, et al. (2018). “CD38-NAD(+)Axis Regulates Immunotherapeutic Anti-Tumor T Cell Response.” Cell Metab 27(1): 85-100 e108.
Cheson, B. D., Fisher, R. I., Barrington, S. F., Cavalli, F., Schwartz, L. H., Zucca, E., and Lister, T. A. (2014). “Recommendations for initial evaluation, staging, and response assessment of Hodgkin and non-Hodgkin lymphoma: the Lugano classification.” J Clin Oncol 32, 3059-3068
Clemens P L, Yan X, Lokhorst H M, Lonial S, Losic N, Khan I, Jansson R, Ahmadi T, Lantz K, Zhou H, Puchalski T, Xu X S. Pharmacokinetics of Daratumumab Following Intravenous Infusion in Relapsed or Refractory Multiple Myeloma After Prior Proteasome Inhibitor and Immunomodulatory Drug Treatment. Clin Pharmacokinet. 2017 August; 56(8):915-924.
Cotner, T., M. Hemler, et al. (1981). “Human T cell proteins recognized by rabbit heteroantisera and monoclonal antibodies.” Int J Immunopharmacol 3(3): 255-268.
Dall'Acqua, W. F., K. E. Cook, et al. (2006). “Modulation of the effector functions of a human IgG1 through engineering of its hinge region.” J Immunol 177(2): 1129-1138.
Damle, R. e. a. (1999). “Ig V gene mutation status and CD38 expression as novel prognostic indicators in chronic lymphocytic leukemia.” Blood 94(6): 1840-1847.
de Jong R N, Beurskens F J, Verploegen S, Strumane K, van Kampen M D, Voorhorst M, Horstman W, Engelberts P J, Oostindie S C, Wang G, Heck A J, Schuurman J, Parren P W. A Novel Platform for the Potentiation of Therapeutic Antibodies Based on Antigen-Dependent Formation of IgG Hexamers at the Cell Surface. PLoS Biol. 2016 Jan. 6; 14(1):e1002344.
de Weers, M., Y. T. Tai, et al. (2011). “Daratumumab, a novel therapeutic human CD38 monoclonal antibody, induces killing of multiple myeloma and other hematological tumors.” J Immunol 186(3): 1840-1848.
Deckert, J., M. C. Wetzel, et al. (2014). “SAR650984, a novel humanized CD38-targeting antibody, demonstrates potent antitumor activity in models of multiple myeloma and other CD38+ hematologic malignancies.” Clin Cancer Res 20(17): 4574-4583.
Deshpande, D. A., T. A. White, et al. (2005). “Altered airway responsiveness in CD38-deficient mice.” Am J Respir Cell Mol Biol 32(2): 149-156.
Desjarlais, J. R. and G. A. Lazar (2011). “Modulation of antibody effector function.” Exp Cell Res 317(9): 1278-1285.
Eissler, N., S. Filosto, et al (2018). “A best in class anti-CD38 antibody with antitumor and immune-modulatory properties.” AACR annual meeting 2018: Abstract #3812.
Feng X., Zhang L., et al. (2017). “Targeting CD38 Suppresses Induction and Function of T Regulatory Cells to Mitigate Immunosuppression in Multiple Myeloma.” Clin Cancer Res 23:4290-4300.
Ho, H. N., L. E. Hultin, et al. (1993). “Circulating HIV-specific CD8+ cytotoxic T cells express CD38 and HLA-DR antigens.” J Immunol 150(7): 3070-3079.
Kaneko, E. and R. Niwa (2011). “Optimizing therapeutic antibody function: progress with Fc domain engineering.” BioDrugs 25(1): 1-11.
Karakasheva T. A., Waldron T. J., et al., (2015). “CD38-Expressing Myeloid-Derived Suppressor Cells Promote Tumor Growth in a Murine Model of Esophageal Cancer.” Cancer Res 75(19):4074-85
Kestens, L., G. Vanham, et al. (1992). “Expression of activation antigens, HLA-DR and CD38, on CD8 lymphocytes during HIV-1 infection.” AIDS 6(8): 793-797.
Keyhani, A., Y. O. Huh, et al. (2000). “Increased CD38 expression is associated with favorable prognosis in adult acute leukemia.” Leuk Res 24(2): 153-159.
Konoplev, S., L. J. Medeiros, et al. (2005). “Immunophenotypic profile of lymphoplasmacytic lymphoma/Waldenstrom macroglobulinemia.” Am J Clin Pathol 124(3): 414-420.
Krejcik, J., T. Casneuf, et al. (2016). “Daratumumab depletes CD38+ immune-regulatory cells, promotes T-cell expansion, and skews T-cell repertoire in multiple myeloma.” Blood 128: 384-394.
Krejcik, J., K. A. Frerichs, et al. (2017). “Monocytes and Granulocytes Reduce CD38 Expression Levels on Myeloma Cells in Patients Treated with Daratumumab.” Clin Cancer Res 23(24): 7498-7511.
Kumar, S., Paiva, B., Anderson, K. C., Durie, B., Landgren, O., Moreau, P., Munshi, N., Lonial, S., Blade, J., Mateos, M. V., et al. (2016). “International Myeloma Working Group consensus criteria for response and minimal residual disease assessment in multiple myeloma.” Lancet Oncol 17, e328-346.
Lammerts van Bueren, J., D. Jakobs, et al. (2014). “Direct in Vitro Comparison of Daratumumab with Surrogate Analogs of CD38 Antibodies MOR03087, SAR650984 and Ab79.” Blood 124(21): 3474.
Lande, R., F. Urbani, et al. (2002). “CD38 ligation plays a direct role in the induction of IL-1beta, IL-6, and IL-10 secretion in resting human monocytes.” Cell Immunol 220(1): 30-38.
Lee, H. C. and R. Aarhus (1993). “Wide distribution of an enzyme that catalyzes the hydrolysis of cyclic ADP-ribose.” Biochim Biophys Acta 1164(1): 68-74.
Lin, P., R. Owens, et al. (2004). “Flow cytometric immunophenotypic analysis of 306 cases of multiple myeloma.” Am J Clin Pathol 121(4): 482-488.
Malavasi, F., A. Funaro, et al. (1994). “Human CD38: a glycoprotein in search of a function.” Immunol Today 15(3): 95-97.
Mallone, R. and P. C. Perin (2006). “Anti-CD38 autoantibodies in type?diabetes.” Diabetes Metab Res Rev 22(4): 284-294.
Marinov, J., K. Koubek, et al. (1993). “Immunophenotypic Significance of the Lymphoid Cd38 Antigen in Myeloid Blood Malignancies.” Neoplasma 40(6): 355-358.
Martin et al., Phase I trial of isatuximab monotherapy in the treatment of refractory multiple myeloma. Blood Cancer J. 2019 Mar. 29; 9(4):41
Morandi F., Horenstein A. L., et al. (2015). “CD56^{bright}CD16⁻ NK Cells Produce Adenosine through a CD38-Mediated Pathway and Act as Regulatory Cells Inhibiting Autologous CD4⁺ T Cell Proliferation.” J Immunol 195:965-972.
Moore, G. L., H. Chen, et al. (2010). “Engineered Fc variant antibodies with enhanced ability to recruit complement and mediate effector functions.” MAbs 2(2): 181-189.
Nijhof I S, Casneuf T, van Velzen J, van Kessel B, Axel A E, Syed K, Groen R W, van Duin M, Sonneveld P, Minnema M C, Zweegman S, Chiu C, Bloem A C, Mutis T, Lokhorst H M, Sasser A K, van de Donk N W (2016) Blood 2016 Aug. 18; 128(7):959-70).
Patton, D. T., Wilson M. D., et al. (2011). “The PI3K p110δ Regulates Expression of CD38 on Regulatory T cells.” PLoS ONE 6(3): 1-8
Parry-Jones, N., E. Matutes, et al. (2007). “Cytogenetic abnormalities additional to t(11; 14) correlate with clinical features in leukaemic presentation of mantle cell lymphoma, and may influence prognosis: a study of 60 cases by FISH.” Br J Haematol 137(2): 117-124.
Perfetti, V., V. Bellotti, et al. (1994). “AL amyloidosis. Characterization of amyloidogenic cells by anti-idiotypic monoclonal antibodies.” Lab Invest 71(6): 853-861.
Raab, M. S., H. Goldschmidt, et al. (2015). “A phase II/IIa study of the human anti-CD38 antibody MOR202 (MOR03087) in relapsed or refractory multiple myeloma (rrMM).” J Clin Oncol 33: A8574.
Rajkumar, Harousseau et al., Consensus recommendations for the uniform reporting of clinical trials: report of the International Myeloma Workshop Consensus Panel, Blood 2011; 117:4691-4695:
Ramaschi, G., M. Torti, et al. (1996). “Expression of cyclic ADP-ribose-synthetizing CD38 molecule on human platelet membrane.” Blood 87(6): 2308-2313.
Roepcke, S., N. Plock, et al. (2018). “Pharmacokinetics and pharmacodynamics of the cytolytic anti-CD38 human monoclonal antibody TAK-079 in monkey—model assisted preparation for the first in human trial.” Pharmacol Res Perspect 6(3): e00402.
Schooten, W. v. (2018). “Multispecific antibodies targeting CD38 and PD-L1 show potent tumor cytotoxicity.” AACR annual meeting 2018: Abstract #5620.
Sondermann, P. and D. E. Szymkowski (2016). “Harnessing Fc receptor biology in the design of therapeutic antibodies.” Curr Opin Immunol 40: 78-87.
Song, A., K. Myojo, et al. (2014). “Evaluation of a fully human monoclonal antibody against multiple influenza A viral strains in mice and a pandemic H1N1 strain in nonhuman primates.” Antiviral Res 111: 60-68.
Suzuki, R., J. Suzumiya, et al. (2004). “Aggressive natural killer-cell leukemia revisited: large granular lymphocyte leukemia of cytotoxic NK cells.” Leukemia 18(4): 763-770.
van de Donk (2018). “Immunomodulatory effects of CD38 targeting antibodies.” Immunology Letters 199:16-22
van de Donk, N. W., M. L. Janmaat, et al. (2016). “Monoclonal antibodies targeting CD38 in hematological malignancies and beyond.” Immunol Rev 270(1): 95-112.
van de Donk, N. W., H. M. Lokhorst, et al. (2012). “How I treat plasma cell leukemia.” Blood 120(12): 2376-2389.
Wang, L., H. Wang, et al. (2015). “CD38 expression predicts poor prognosis and might be a potential therapy target in extranodal NK/T cell lymphoma, nasal type.” Ann Hematol 94(8): 1381-1388.
Wang, X., M. Mathieu, et al. (2018). “IgG Fc engineering to modulate antibody effector functions.” Protein & Cell 9(1): 63-73.
Zhang, D., A. A. Armstrong, et al. (2017). “Functional optimization of agonistic antibodies to OX40 receptor with novel Fc mutations to promote antibody multimerization.” MAbs 9(7): 1129-1142.
Zocchi, E., L. Franco, et al. (1993). “A single protein immunologically identified as CD38 displays NAD+ glycohydrolase, ADP-ribosyl cyclase and cyclic ADP-ribose hydrolase activities at the outer surface of human erythrocytes.” Biochem Biophys Res Commun 196(3): 1459-1465.
Nijhof et al., Blood 2016; 128(7):959-970
“Supplemental Methods” to Nijhof et al., 2016
WO 2006/099875 A1 (Genmab A/S)
WO 2007/042309 A1 (Morphosys AG)
WO 2008/047242 A1 (Sanofi Aventis)
WO 2011/154453 A1 (Genmab A/S)
WO 2012/092612 A1 (Takeda Pharmaceutical)
WO 2013/004842 A2 (Genmab A/S)
WO 2014/108198 A1 (Genmab B.V.)
WO 2016/210223 A1 (Janssen Biotech, Inc.)
WO 2018/031258 A1 (Janssen Biotech, Inc.)
WO 2020/012036 A1 (Genmab A/S)
WO 2020/012038 A1 (Genmab A/S)
WO 2021/144457 A1 (Genmab A/S)

### EXAMPLES

The present invention is further illustrated by the following examples which should not be construed as limiting.

### Example 1—Antibodies Production and Formulation

### Antibody Expression Constructs

Antibodies were essentially produced as described in WO 2020/012036 A1, WO 2020/012038 A1, WO 2021/144457 A1 (all incorporated herein by reference).

In short, for the expression of human and humanized antibodies used herein, variable heavy (VH) chain and variable light (VL) chain sequences were prepared by gene synthesis (GeneArt Gene Synthesis; ThermoFisher Scientific) and cloned in pcDNA3.3 expression vectors (ThermoFisher Scientific) containing a constant region of a human IgG heavy chain (HC) (constant region human IgG1 m(f) HC: SEQ ID NO:20) and/or the constant region of the human kappa light chain (LC): SEQ ID NO:37. Desired mutations were introduced by gene synthesis. CD38 antibody variants described herein have VH and VL sequences derived from previously described CD38 antibodies IgG1-A (WO 2006/099875 A1, WO 2008/037257 A2, WO 2011/154453 A1; VH: SEQ ID NO:10; VL: SEQ ID NO:11), IgG1-B (WO 2006/099875 A1, WO 2008/037257 A2, WO 2011/154453 A1; VH: SEQ ID NO:8; VL: SEQ ID NO:9), and IgG1-C(WO 2011/154453 A1; VH: SEQ ID NO:1; VL: SEQ ID NO:5). The human IgG1 antibody b12, an HIV gp120-specific antibody was used as a negative control in some experiments (Barbas et al., J Mol Biol. 1993 Apr. 5; 230(3):812-23; VH: SEQ ID NO:12; VL: SEQ ID NO:16).

### Transient Expression Antibody Constructs

Plasmid DNA mixtures encoding both heavy and light chains of antibodies were transiently transfected in Expi293F cells (Gibco, Cat No A14635) using 293fectin (Life Technologies) essentially as described by Vink et al. (Vink et al., 2014 Methods 65(1):5-10). Antibody concentrations in the supernatants were measured by absorbance at 280 nm. Antibody-containing supernatants were either directly used in in vitro assays, or antibodies were purified as described below.

### Antibody Purification and Quality Assessment

Antibodies were purified by Protein A affinity chromatography. Culture supernatants were filtered over a 0.20 μM dead-end filter and loaded on 5 mL MabSelect SuRe columns (GE Healthcare), washed and eluted with 0.02 M sodium citrate-NaOH, pH 3. The eluates were loaded on a HiPrep Desalting column (GE Healthcare) immediately after purification and the antibodies were buffer exchanged into 12.6 mM NaH2PO4, 140 mM NaCl, pH 7.4 buffer (B. Braun or Thermo Fisher). After buffer exchange, samples were sterile filtered over 0.2 μm dead-end filters. Purified proteins were analyzed by a number of bioanalytical assays including capillary electrophoresis on sodium dodecyl sulfate-polyacrylamide gels (CE-SDS) and high-performance size exclusion chromatography (HP-SEC). Concentration was measured by absorbance at 280 nm. Purified antibodies were stored at 2-8° C.

IgG1-C-E430G comprises a VH comprising SEQ ID NO:1, a VL comprising SEQ ID NO:5, a CH comprising SEQ ID NO:46 and a CL comprising SEQ ID NO:37. IgG1-C-E430G can be expressed in CHO cells.

### Antibody Formulation

Antibody IgG1-C-E430G as used in the trial as described in the Examples hereunder was essentially produced and formulated as described in WO 2021/144457 A1 (incorporated herein by reference). The optimal formulation for IgG1-C-E430G was found to be 20 mM histidine, 250 mM sorbitol, 0.04% (w/v) PS80, pH 6.0. The formulation comprised 20 mg/ml of the antibody.

### Example 2—Clinical Trial

### Trial Design:

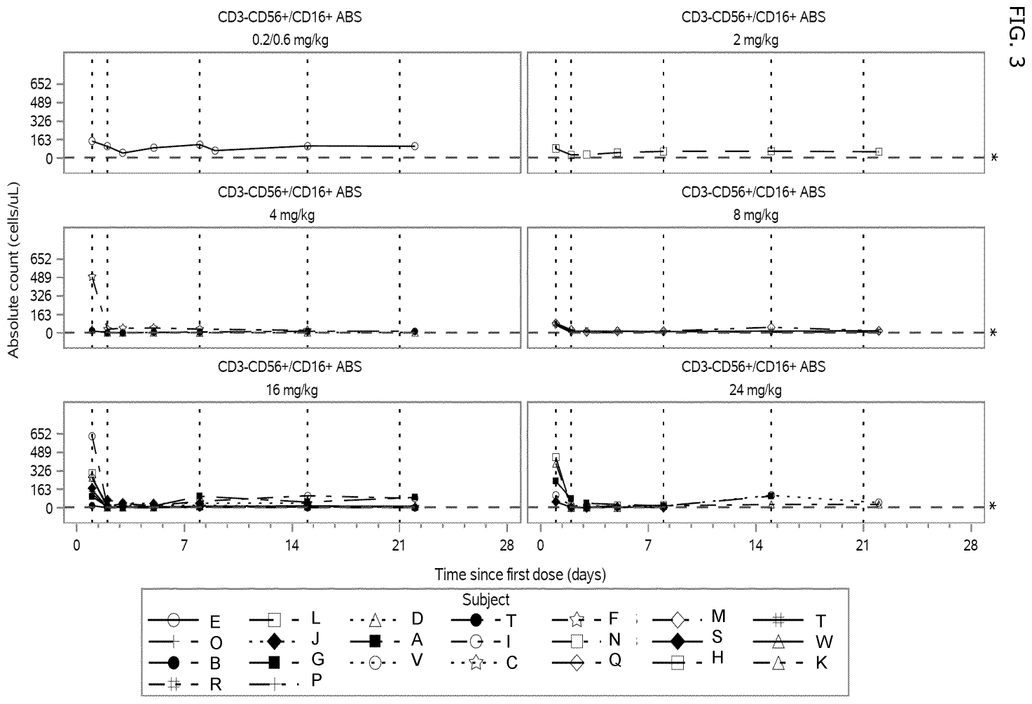

An open-label, multicenter, phase 1/2 trial was conducted of IgG1-C-E430G to evaluate the safety, tolerability, PK, pharmacodynamics, immunogenicity, and preliminary efficacy of IgG1-C-E430G in subjects with RRMM and other hematologic malignancies including R/R DLBCL. The trial consists of 3 parts: dose escalation (phase 1), expansion part A (IgG1-C-E430G single cohorts) (phase 2), and expansion part B (randomized head-to-head) (phase 2). FIG. 2 shows a schematic representation of the clinical trial design.

### Dose Escalation

The dose escalation part was designed to evaluate IgG1-C-E430G in subjects with RRMM to determine the recommended phase 2 dose (RP2D).

IgG1-C-E430G was evaluated at 6 dose levels in subjects with RRMM. The Cycle 1 first dose was split into 2 doses administered on consecutive days. Table 5 shows the IgG1-C-E430G dose administration in subjects with RRMM.

TABLE 5
IgG1-C-E430G Dose Administration - Subjects With RRMM (Dose Escalation)

| | Minimum | | | Dose Administered Each |
|---|---|---|---|---|
| Subjects | Number of | | | Dosing Day, Cycle 2 and |
| With MM | Subjects | Dose Level | Dose Administered in Cycle 1 | Beyond |
| MM-DL1 | 1 | 0.2 to 0.6 mg/kg | Days 1 and 2: 0.1 mg/kg/day | 0.6 mg/kg |
| | | (intrasubject dose | Days 8 and 9: 0.3 mg/kg/day | |
| | | escalation) | Day 15: 0.6 mg/kg | |
| | | | Day 22: 0.6 mg/kg | |
| MM-DL2 | 1 | 2 mg/kg | Days 1 and 2: 1 mg/kg/day | 2 mg/kg |
| | | | Day 8: 2 mg/kg | |
| | | | Day 15: 2 mg/kg | |
| | | | Day 22: 2 mg/kg | |
| MM-DL3 | 3 | 4 mg/kg | Days 1 and 2: 2 mg/kg/day | 4 mg/kg |
| | | | Day 8: 4 mg/kg | |
| | | | Day 15: 4 mg/kg | |
| | | | Day 22: 4 mg/kg | |
| MM-DL4 | 3 | 8 mg/kg | Days 1 and 2: 4 mg/kg/day | 8 mg/kg |
| | | | Day 8: 8 mg/kg | |
| | | | Day 15: 8 mg/kg | |
| | | | Day 22: 8 mg/kg | |
| MM-DL5 | 3 | 16 mg/kg | Days 1 and 2: 8 mg/kg/day | 16 mg/kg |
| | | | Day 8: 16 mg/kg | |
| | | | Day 15: 16 mg/kg | |
| | | | Day 22: 16 mg/kg | |
| MM-DL 6 | 3 | 24 mg/kg | Days 1 and 2: 12 mg/kg/day | 24 mg/kg |
| | | | Day 8: 24 mg/kg | |
| | | | Day 15: 24 mg/kg | |
| | | | Day 22: 24 mg/kg | |

Note:
For MM-DL1, split doses are administered on Days 1-2 and Days 8-9 of Cycle 1; full doses are administered on subsequent dosing days. For all other MM-DL cohorts, split doses are administered only on Days 1-2 of Cycle 1; full doses are administered on subsequent dosing days.
Additional DL such as 20 mg/kg or modified dosing schedule(s) may also be explored based upon emerging data.
DL = dose level; RRMM = relapsed or refractory multiple myeloma; MM = multiple myeloma.

IgG1-C-E430G was administered in the RRMM cohort as an IV infusion in cycles of 4 weeks, ie, 28 days, as follows:
Cycle 1: Days 1, 2, 8, 15, and 22 (Q1W). Note that the first dose of IgG1-C-E430G was split into 2 consecutive days (i.e., C1D1 and C1 D2) and that for RRMM, MM-DL1 was dosed on Day 9.
Cycle 2: Days 1, 8, 15, and 22 (Q1W)
Cycles 3-6: Days 1 and 15 (Q2W)
Cycle 7 and beyond: Day 1 (Q4W)

Each infusion was estimated to last between 1 and 8 hours.

FIG. 2 shows a schematic overview of the IgG1-C-E430G trial design.

### Expansion Part A

The aim of expansion part A is to provide further data on the efficacy, safety, tolerability, pharmacokinetics, pharmacodynamics, and biomarkers.

Expansion part A is designed to include subjects with RRMM and R/R DLBCL in cohorts as follows:
Data inclusive of the first 10 response-evaluable subjects, those anti-CD38 mAb-naive subjects with RRMM who received IgG1-C-E430G at 16 mg/kg or 24 mg/kg from the dose escalation and/or from the expansion part A
Twenty subjects with anti-CD38 mAb-refractory RRMM
Up to 40 subjects with R/R DLBCL

### Expansion Part B

In Expansion Part B (randomized H2H), IgG1-C-E430G IV is compared to a reference anti CD38 antibody (without E340G, i.e. subcutaneous (SC) daratumumab—DARZALEX FASPRO®) in anti-CD38 mAb-naive RRMM subjects.

### Demographics

Subjects are eligible to be included in the trial only if all of the following criteria apply:

Subject must be at least 18 years of age.

Subjects with RRMM

Subjects must have documented multiple myeloma as defined by the criteria below and have evidence of disease progression on the most recent prior treatment regimen based on IMWG criteria:
Prior documentation of monoclonal plasma cells in the bone marrow ≥10% or presence of a biopsy-proven plasmacytoma.
   and
Measurable disease at baseline as defined by any of the following:
   IgG, IgA, IgD, or IgM myeloma: serum M-protein level ≥0.5 g/dL (≥5 g/L) or urine M-protein level ≥200 mg/24 hours;
      or
Light chain myeloma: serum Ig free light chain (FLC) ≥10 mg/dL and abnormal serum Ig kappa lambda FLC ratio.

Subjects must have exhausted standard therapies, at the investigator's discretion.

Subjects with R/R DLBCL

Subjects may have either de novo or histologically transformed DLBCL. Subjects with R/R DLBCL must have exhausted standard therapies, at the investigator's discretion.

### Exposure

The median duration of IgG1-C-E430G full dose infusion was 3.80 hours (range: 1.15-4.57 hours).

### Example 3—Clinical Efficacy

### Results

Table 6 shows best overall response by dose level among 21 response-evaluable subjects with RRMM (data cut-off date: 3 Oct. 2022). Preliminary anti-tumor activity was observed during the dose escalation part of the IgG-C-E430G trial at 4 mg/kg and higher DLs. At 16 mg/kg DL, among 9 response-evaluable subjects, 3 subjects were anti-CD38 naive and 1 subject had minimal response; 6 subjects were previously exposed to anti-CD38 antibodies and 1 subject each had partial response and minimal response (Table 7 and Table 8).

Table 7 shows best overall response by dose level among 5 anti-CD38 mAb-naive subjects who were response-evaluable. Two subjects achieved complete response (CR; 1 subject at 4 mg/kg and 1 subject at 24 mg/kg), and 1 subject had minimal response (MR) at 16 mg/kg.

Table 8 shows best overall response by dose level among 16 ani-CD38 mAb-treated subjects. The best response observed was partial response (PR) in 1 subject at 16 mg/kg; additionally, 2 subjects achieved MR (1 subject at 8 mg/kg and 1 subject at 16 mg/kg).

In summary, data from the dose escalation part of the IgG-C-E430G trial indicate that IgG1-C-E430G has promising clinical activity in patients with anti-CD38 mAb-naive RRMM. Anti-CD38 mAb-pretreated RRMM patients, such as heavily pretreated patients with RRMM who have received proteasome inhibitors, immunomodulatory drugs, and anti-CD38 mAb treatment may potentially benefit from the IgG1-C-E430G treatment as well.

TABLE 6
Best Overall Response - All RRMM

| | 0.2/0.6 mg/kg | 2 mg/kg | 4 mg/kg | 8 mg/kg | 16 mg/kg | 24 mg/kg | Total |
|---|---|---|---|---|---|---|---|
| | (N = 1) | (N = 1) | (N = 3) | (N = 3) | (N = 9) | (N = 4) | (N = 21) |
| Best overall response, | | | | | | | |
| n(%) | | | | | | | |
| Stringent complete | 0 | 0 | 0 | 0 | 0 | 0 | 0 |
| response | | | | | | | |
| Complete response | 0 | 0 | 1 | 0 | 0 | 1 | 2 |
| | | | (33.3%) | | | (25.0%) | (9.5%) |
| Very good partial | 0 | 0 | 0 | 0 | 0 | 0 | 0 |
| response | | | | | | | |
| Partial response | 0 | 0 | 0 | 0 | 1 | 0 | 1 |
| | | | | | (11.1%) | | (4.8%) |
| Minimal response | 0 | 0 | 0 | 1 | 2 | 0 | 3 |
| | | | | (33.3%) | (22.2%) | | (14.3%) |
| Stable disease | 1 | 1 | 2 | 2 | 4 | 1 | 11 |
| | (100%) | (100%) | (66.7%) | (66.7%) | (44.4%) | (25.0%) | (52.4%) |
| Progressive disease | 0 | 0 | 0 | 0 | 2 | 2 | 4 |
| | | | | | (22.2%) | (50.0%) | (19.0%) |
| Not evaluable | 0 | 0 | 0 | 0 | 0 | 0 | 0 |
| CR or better | 0 | 0 | 1 | 0 | 0 | 1 | 2 |
| | | | (33.3%) | | | (25.0%) | (9.5%) |
| (95% CI) [a] | (0%, | (0%, | (0.6%, | (0% | (0%, | (0.6%, | (1.2%, |
| | 97.5%) | 97.5%) | 90.6%) | 70.8%) | 33.6%) | (80.6%) | 30.4%) |
| VGPR or better | 0 | 0 | 1 | 0 | 0 | 1 | 2 |
| | | | (33.3%) | | | (25.0%) | (9.5%) |
| (95% CI) [a] | (0%, | (0%, | (0.8%, | (0%, | (0%, | (0.6%, | (1.2%, |
| | 97.5%) | 97.5%) | 90.6%) | 70.8%) | 33.6%) | 80.6%) | 30.4%) |
| Overall response rate | 0 | 0 | 1 | 0 | 1 | 1 | 3 |
| (ORR) [b] | | | (33.3%) | | (11.1%) | (25.0%) | (14.3%) |
| (95% CI) [a] | (0%, | (0%, | (0.8%, | (0% | (0.3%, | (0.6%, | (3.0%, |
| | 97.5%) | 97.5%) | 90.6%) | 70.8%) | 48.2%) | 80.6%) | 36.3%) |
| Clinical benefit [b] | 0 | 0 | 1 | 1 | 3 | 1 | 6 |
| | | | (33.3%) | (33.3%) | (33.3%) | (25.0%) | (28.6%) |
| (95% CI) [a] | (0%, | (0%, | (0.8%, | (0.8%, | (7.5%, | (0.6%, | (11.3%, |
| | 97.5%) | 97.5%) | 90.6%) | 90.6%) | 70.1%) | 80.6%) | 52.2%) |

[a] Based on the Clopper and Pearson method.
[b] ORR includes subjects with best response of CR and PR; Clinical benefit includes ORR and MR.

TABLE 7
Best Overall Response - Anti-CD38 mAb-naive RRMM

| | 0.2/0.6 mg/kg | 2 mg/kg | 4 mg/kg | 8 mg/kg | 16 mg/kg | 24 mg/kg | Total |
|---|---|---|---|---|---|---|---|
| Best overall response, n(%) | (N = 0) | (N = 0) | (N = 1) | (N = 0) | (N = 3) | (N = 1) | (N = 5) |
| Stringent complete response | z | z | 0 | z | 0 | 0 | 0 |
| Complete response | | | 1 (100%) | | 0 | 1 (100%) | 2 (40.0%) |
| Very good partial response | | | 0 | | 0 | 0 | 0 |
| Partial response | | | 0 | | 0 | 0 | 0 |
| Minimal response | | | 0 | | 1 (33.3%) | 0 | 1 (20.0%) |
| Stable disease | | | 0 | | 2 (66.7%) | 0 | 2 (40.0%) |
| Progressive disease | | | 0 | | 0 | 0 | 0 |
| Not evaluable | | | 0 | | 0 | 0 | 0 |
| CR or better | | | 1 (100%) | | 0 | 1 (100%) | 2 (40.0%) |
| (95% CI) [a] | | | (2.5%, 100%) | | (0%, 70.8%) | (2.5%, 100%) | (5.3%, 85.3%) |
| VGPR or better | | | 1 (100%) | | 0 | 1 (100%) | 2 (40.0%) |
| (95% CI) [a] | | | (2.5%, 100%) | | (0%, 70.8%) | (2.5%, 100%) | (5.3%, 85.3%) |
| Overall response rate (ORR) [b] | | | 1 (100%) | | 0 | 1 (100%) | 2 (40.0%) |
| (95% CI) [a] | | | (2.5%, 100%) | | (0%, 70.8%) | (2.5%, 100%) | (5.3%, 85.3%) |
| Clinical benefit [b] | | | 1 (100%) | | 1 (33.3%) | 1 (100%) | 3 (60.0%) |
| (95% CI) [a] | | | (2.5%, 100%) | | (0.8%, 90.6%) | (2.5%, 100%) | (14.7%, 94.7%) |

[a] Based on the Clopper and Pearson method.
[b] ORR includes subjects with best response of CR and PR; Clinical benefit includes ORR and MR.

TABLE 8
Best Overall Response - Anti-CD38 mAb-treated RRMM

| | 0.2/0.6 mg/kg | 2 mg/kg | 4 mg/kg | 8 mg/kg | 16 mg/kg | 24 mg/kg | Total |
|---|---|---|---|---|---|---|---|
| Best overall response, n(%) | (N = 1) | (N = 1) | (N = 2) | (N = 3) | (N = 6) | (N = 3) | (N = 16) |
| Stringent complete response | 0 | 0 | 0 | 0 | 0 | 0 | 0 |
| Complete response | 0 | 0 | 0 | 0 | 0 | 0 | 0 |
| Very good partial response | 0 | 0 | 0 | 0 | 0 | 0 | 0 |
| Partial response | 0 | 0 | 0 | 0 | 1 (16.7%) | 0 | 1 (6.3%) |
| Minimal response | 0 | 0 | 0 | 1 (33.3%) | 1 (16.7%) | 0 | 2 (12.5%) |
| Stable disease | 1 (100%) | 1 (100%) | 2 (100%) | 2 (66.7%) | 2 (33.3%) | 1 (33.3%) | 9 (56.3%) |
| Progressive disease | 0 | 0 | 0 | 0 | 2 (33.3%) | 2 (66.7%) | 4 (25.0%) |
| Not evaluable | 0 | 0 | 0 | 0 | 0 | 0 | 0 |
| CR or better | 0 | 0 | 0 | 0 | 0 | 0 | 0 |
| (95% CI) [a] | (0%, 97.5%) | (0%, 97.5%) | (0%, 84.2%) | (0%, 70.8%) | (0%, 45.9%) | (0%, 70.8%) | (0%, 20.6%) |
| VGPR or better | 0 | 0 | 0 | 0 | 0 | 0 | 0 |
| (95% CI) [a] | (0%, 97.5%) | (0%, 97.5%) | (0%, 84.2%) | (0%, 70.8%) | (0%, 45.9%) | (0%, 70.8%) | (0%, 20.6%) |
| Overall response rate (ORR) [b] | 0 | 0 | 0 | 0 | 1 (16.7%) | 0 | 1 (6.3%) |
| (95% CI) [a] | (0%, 97.5%) | (0%, 97.5%) | (0%, 84.2%) | (0%, 70.8%) | (0.4%, 64.1%) | (0%, 70.8%) | (0.2%, 30.2%) |
| Clinical benefit [b] | 0 | 0 | 0 | 1 (33.3%) | 2 (33.3%) | 0 | 3 (18.8%) |
| (95% CI) [a] | (0%, 97.5%) | (0%, 97.5%) | (0%, 84.2%) | (0.8%, 90.6%) | (4.3%, 77.7%) | (0%, 70.8%) | (4.0%, 45.6%) |

[a] Based on the Clopper and Pearson method.
[b] ORR includes subjects with best response of CR and PR; Clinical benefit includes ORR and MR.

### Example 4—Clinical Safety

### Results

As of 3 Oct. 2022, all 24 subjects (100%) in the dose escalation part experienced at least 1 treatment-emergent adverse event (TEAE); 21 subjects (87.5%) experienced a TEAE considered as related to IgG1-C-E430G (Table 9 and Table 10). The most common TEAEs (in ≥20% of subjects) include IRR (18 subjects; 75.0%), neutropenia (15 subjects; 62.5%), diarrhea (10 subjects; 41.7%), anemia (10 subjects; 41.7%), COVID-19 (6 subjects; 25.0%), pyrexia (5 subjects; 20.8%), thrombocytopenia (5 subjects; 20.8%), and vision blurred (5 subjects; 20.8%); refer to Table 9.

Serious TEAEs were reported in 15 (62.5%) subjects (Table 11). The most common serious TEAEs (in 5% of subjects) include COVID-19 (3 subjects; 12.5%), pyrexia (3 subjects; 12.5%), IRR (2 subjects; 8.3%), and pneumonia (2 subjects; 8.3%). Four subjects (16.7%) experienced a serious TEAE that was considered related to IgG1-C-E430G: IRR in 2 subjects (8.3%), appendicitis, neutropenic sepsis, and fungal pneumonia in 1 subject (4.2%), and increased fibrin D dimer in 1 subject (4.2%); refer to Table 12.

Eighteen subjects (75.0%) experienced IRR at DLs of 4, 8, 16, and 24 mg/kg (Table 6).

No cytokine release syndrome events were reported. There were no fatal TEAEs. All subjects in the immunogenicity analysis set were negative for anti-drug antibodies.

Twenty-one subjects (87.5%) discontinued trial treatment and 3 subjects (12.5%) continue to receive IgG1-C-E430G. Reasons for treatment discontinuation include progressive disease (13 subjects; 54.2%), AE (4 subjects; 16.7%), clinical progression (3 subjects; 12.5%), and subject request to discontinue trial treatment (1 subject; 4.2%). The AEs leading to treatment discontinuation included IRR (2 subjects; 8.3%), neutropenia (1 subject; 4.2%), neutropenic sepsis (1 subject; 4.2%), respiratory syncytial virus infection (1 subject; 4.2%), and thrombocytopenia (1 subject; 4.2%).

As of 3 Oct. 2022, the totality of the data indicates that the 16 mg/kg DL would result in clinical activity while ensuring a tolerable safety profile; therefore, 16 mg/kg was selected as the RP2D for the expansion cohorts in RRMM and R/R DLBCL in the IgG-C-E430G trial.

Clinical safety data from subjects with RRMM from the dose escalation part of the IgG-C-E430G trial demonstrated that IgG1-C-E430G was well tolerated in the 11 subjects treated at the 16 mg/kg DL.
Among 11 subjects treated at 16 mg/kg, the most common TEAEs (20%) included IRR (9 subjects, 81.8%; all related), neutropenia (7 subjects, 63.6%; 6 related), diarrhea (5 subjects, 45.5%; 2 related), fatigue (3 subjects, 27.3%; 2 related), vision blurred (3 subjects, 27.3%; 1 related), and COVID-19 (3 subjects, 27.3%; none related); refer to Table 9 and Table 10.
At the 16 mg/kg DL, serious TEAEs were reported in 6 subjects (54.5%; Table 11). One subject each (9.1%) experienced the following serious TEAEs that were considered unrelated to IgG1-C-E430G: COVID-19, pneumonia, dyspnea, rhinovirus infection, acute myocardial infarction, hypervolemia, and pneumococcal sepsis. One subject (9.1%) experienced a serious TEAE that was considered related to IgG1-C-E430G, IRR; refer to Table 12.
At the 16 mg/kg DL, 10 subjects (90.9%) have discontinued trial treatment and 1 subject (9.1%) continue to receive IgG1-C-E430G. Reasons for treatment discontinuation include progressive disease (5 subjects; 45.5%), clinical progression (3 subjects, 27.3%) and AE (2 subjects, 18.2%; both due to IRR). The median duration on treatment was 2.5 months (range: 0.2-5.1 months). IgG1-C-E430G.

In summary, data from the dose escalation part of the IgG-C-E430G trial suggest that (IgG1-C-E430G has an acceptable safety profile, with no tumor lysis syndrome or cytokine release syndrome events and no on-treatment deaths.

TABLE 9
Treatment Emergent Adverse Events

| | 0.2/0.6 mg/kg | 2 mg/kg | 4 mg/kg | 8 mg/kg | 16 mg/kg | 24 mg/kg | Total |
|---|---|---|---|---|---|---|---|
| | (N = 1) | (N = 1) | (N = 3) | (N = 3) | (N = 11) | (N = 5) | (N = 24) |
| Number of subjects with | 1 (100%) | 1 (100%) | 3 (100%) | 3 (100%) | 11 (100%) | 5 (100%) | 24 (100%) |
| at least one TEAE, n(%) | | | | | | | |
| Infusion related reaction | 0 | 0 | 2 (66.7%) | 3 (100%) | 9 (81.8%) | 4 (80.0%) | 18 (75.0%) |
| Neutropenia | 0 | 0 | 3 (100%) | 2 (66.7%) | 7 (63.6%) | 3 (60.0%) | 15 (62.5%) |
| Diarrhoea | 0 | 0 | 1 (33.3%) | 1 (33.3%) | 5 (45.5%) | 3 (60.0%) | 10 (41.7%) |
| Anaemia | 1 (100%) | 1 (33.3%) | 1 (33.3%) | 0 | 3 (27.3%) | 4 (80.0%) | 10 (41.7%) |
| COVID-19 | 0 | 0 | 0 | 2 (66.7%) | 3 (27.3%) | 1 (20.0%) | 6 (25.0%) |
| Pyrexia | 1 (100%) | 0 | 2 (66.7%) | 1 (33.3%) | 1 (9.1%) | 1 (20.0%) | 6 (25.0%) |
| Thrombocytopenia | 0 | 0 | 1 (33.3%) | 0 | 2 (18.2%) | 2 (40.0%) | 5 (20.8%) |
| Vision blurred | 0 | 1 (100%) | 0 | 1 (33.3%) | 3 (27.3%) | 0 | 5 (20.8%) |
| Fatigue | 0 | 0 | 0 | 0 | 3 (27.3%) | 1 (20.0%) | 4 (16.7%) |
| Hypokalaemia | 0 | 0 | 1 (33.3%) | 0 | 1 (9.1%) | 2 (40.0%) | 4 (16.7%) |
| Hypophosphataemia | 0 | 0 | 0 | 0 | 2 (18.2%) | 2 (40.0%) | 4 (16.7%) |
| Nausea | 1 (100%) | 0 | 0 | 0 | 2 (18.2%) | 1 (20.0%) | 4 (16.7%) |
| Arthralgia | 0 | 0 | 1 (33.3%) | 2 (66.7%) | 0 | 0 | 3 (12.5%) |
| Conjunctival oedema | 0 | 0 | 0 | 1 (33.3%) | 1 (9.1%) | 1 (20.0%) | 3 (12.5%) |
| Fibrin D dimer increased | 0 | 0 | 0 | 3 (100%) | 0 | 0 | 3 (12.5%) |
| Headache | 0 | 0 | 0 | 1 (33.3%) | 0 | 2 (40.0%) | 3 (12.5%) |
| Insomnia | 1 (100%) | 0 | 0 | 0 | 2 (18.2%) | 0 | 3 (12.5%) |
| Vomiting | 1 (100%) | 0 | 0 | 0 | 2 (18.2%) | 0 | 3 (12.5%) |
| Back pain | 0 | 0 | 0 | 0 | 1 (9.1%) | 1 (20.0%) | 2 (8.3%) |
| Bone pain | 0 | 0 | 0 | 0 | 0 | 2 (40.0%) | 2 (8.3%) |
| Dizziness | 0 | 0 | 0 | 0 | 2 (18.2%) | 0 | 2 (8.3%) |
| Febrile neutropenia | 0 | 0 | 0 | 0 | 0 | 2 (40.0%) | 2 (8.3%) |
| Flank pain | 0 | 0 | 0 | 0 | 1 (9.1%) | 1 (20.0%) | 2 (8.3%) |
| Leukopenia | 0 | 0 | 0 | 0 | 2 (18.2%) | 0 | 2 (8.3%) |
| Oedema peripheral | 0 | 0 | 1 (33.3%) | 0 | 0 | 1 (20.0%) | 2 (8.3%) |
| Oral candidiasis | 0 | 0 | 1 (33.3%) | 0 | 0 | 1 (20.0%) | 2 (8.3%) |
| Oropharyngeal pain | 0 | 0 | 1 (33.3%) | 1 (33.3%) | 0 | 0 | 2 (8.3%) |
| Pneumonia | 0 | 1 (100%) | 0 | 0 | 1 (9.1%) | 0 | 2 (8.3%) |
| Syncope | 0 | 0 | 0 | 0 | 2 (18.2%) | 0 | 2 (8.3%) |
| Urinary tract infection | 0 | 0 | 0 | 0 | 1 (9.1%) | 1 (20.0%) | 2 (8.3%) |
| Vaccination complication | 0 | 0 | 0 | 1 (33.3%) | 1 (9.1%) | 0 | 2 (8.3%) |
| Abdominal distension | 0 | 0 | 0 | 0 | 0 | 1 (20.0%) | 1 (4.2%) |
| Abdominal pain | 0 | 0 | 0 | 0 | 0 | 1 (20.0%) | 1 (4.2%) |
| Acute myocardial infarction | 0 | 0 | 0 | 0 | 1 (9.1%) | 0 | 1 (4.2%) |
| Adenoviral upper respiratory | 0 | 0 | 0 | 0 | 0 | 1 (20.0%) | 1 (4.2%) |
| infection | | | | | | | |
| Alanine aminotransferase | 0 | 0 | 1 (33.3%) | 0 | 0 | 0 | 1 (4.2%) |
| increased | | | | | | | |
| Anxiety | 0 | 0 | 1 (33.3%) | 0 | 0 | 0 | 1 (4.2%) |
| Appendicitis | 0 | 0 | 0 | 0 | 0 | 1 (20.0%) | 1 (4.2%) |
| Aspartate aminotransferase | 0 | 0 | 0 | 0 | 1 (9.1%) | 0 | 1 (4.2%) |
| increased | | | | | | | |
| Atrial fibrillation | 0 | 0 | 0 | 0 | 0 | 1 (20.0%) | 1 (4.2%) |
| Bacteraemia | 0 | 0 | 0 | 0 | 1 (9.1%) | 0 | 1 (4.2%) |
| Blood creatinine increased | 1 (100%) | 0 | 0 | 0 | 0 | 0 | 1 (4.2%) |
| Blood folate decreased | 0 | 0 | 0 | 0 | 0 | 1 (20.0%) | 1 (4.2%) |
| Blood phosphorus decreased | 0 | 0 | 0 | 0 | 1 (9.1%) | 0 | 1 (4.2%) |
| Catheter site haemorrhage | 0 | 0 | 0 | 0 | 0 | 1 (20.0%) | 1 (4.2%) |
| Chest pain | 0 | 0 | 0 | 0 | 1 (9.1%) | 0 | 1 (4.2%) |
| Clavicle fracture | 0 | 1 (100%) | 0 | 0 | 0 | 0 | 1 (4.2%) |
| Coccydynia | 0 | 0 | 0 | 1 (33.3%) | 0 | 0 | 1 (4.2%) |
| Conjunctival haemorrhage | 0 | 0 | 0 | 1 (33.3%) | 0 | 0 | 1 (4.2%) |
| Conjunctivitis | 0 | 0 | 0 | 1 (33.3%) | 0 | 0 | 1 (4.2%) |
| Contusion | 0 | 1 (100%) | | 0 | 0 | 0 | 1 (4.2%) |
| Cough | 0 | 0 | 0 | 0 | 1 (9.1%) | 0 | 1 (4.2%) |
| Cutaneous vasculitis | 0 | 0 | 1 (33.3%) | 0 | 0 | 0 | 1 (4.2%) |
| Cystitis | 0 | 0 | 0 | 0 | 1 (9.1%) | 0 | 1 (4.2%) |
| Decreased appetite | 0 | 0 | 1 (33.3%) | 0 | 0 | 0 | 1 (4.2%) |
| Delirium | 0 | 0 | 0 | 0 | 0 | 1 (20.0%) | 1 (4.2%) |
| Device leakage | 0 | 0 | 0 | 0 | 0 | 1 (20.0%) | 1 (4.2%) |
| Dry skin | 0 | 0 | 0 | 0 | 0 | 1 (20.0%) | 1 (4.2%) |
| Dyspepsia | 0 | 0 | 0 | 0 | 0 | 1 (20.0%) | 1 (4.2%) |
| Dyspnoea | 0 | 0 | 0 | 0 | 1 (9.1%) | 0 | 1 (4.2%) |
| Epistaxis | 0 | 0 | 1 (33.3%) | 0 | 0 | 0 | 1 (4.2%) |
| Fall | 0 | 0 | 1 (33.3%) | 0 | 0 | 0 | 1 (4.2%) |
| Gallbladder obstruction | 0 | 0 | 0 | 0 | 1 (9.1%) | 0 | 1 (4.2%) |
| Gingival pain | 0 | 0 | 0 | 0 | 1 (9.1%) | 0 | 1 (4.2%) |
| Haematoma | 0 | 0 | 0 | 0 | 1 (9.1%) | 0 | 1 (4.2%) |
| Hypercalcaemia | 0 | 0 | 0 | 0 | 0 | 1 (20.0%) | 1 (4.2%) |
| Hypervolaemia | 0 | 0 | 0 | 0 | 1 (9.1%) | 0 | 1 (4.2%) |
| Hyponatraemia | 0 | 0 | 1 (33.3%) | 0 | 0 | 0 | 1 (4.2%) |
| Hypotension | 0 | 0 | 0 | 0 | 0 | 1 (20.0%) | 1 (4.2%) |
| Influenza | 0 | 0 | 0 | 1 (33.3%) | 0 | 0 | 1 (4.2%) |
| Lymphopenia | 0 | 0 | 1 (33.3%) | 0 | 0 | 0 | 1 (4.2%) |
| Metapneumovirus infection | 0 | 0 | 0 | 0 | 1 (9.1%) | 0 | 1 (4.2%) |
| Mouth haemorrhage | 0 | 0 | 0 | 0 | 0 | 1 (20.0%) | 1 (4.2%) |
| Muscle spasms | 0 | 0 | 1 (33.3%) | 0 | 0 | 0 | 1 (4.2%) |
| Muscular weakness | 1 (100%) | 0 | 0 | 0 | 0 | 0 | 1 (4.2%) |
| Musculoskeletal pain | 0 | 0 | 0 | 0 | 1 (9.1%) | 0 | 1 (4.2%) |
| Myalgia | 0 | 0 | 1 (33.3%) | 0 | 0 | 0 | 1 (4.2%) |
| Nasopharyngitis | 0 | 0 | 1 (33.3%) | 0 | 0 | 0 | 1 (4.2%) |
| Neck pain | 0 | 0 | 1 (33.3%) | 0 | 0 | 0 | 1 (4.2%) |
| Neutropenic sepsis | 0 | 0 | 0 | 0 | 0 | 1 (20.0%) | 1 (4.2%) |
| Oesophageal candidiasis | 0 | 0 | 0 | 1 (33.3%) | 0 | 0 | 1 (4.2%) |
| Oral pain | 0 | 0 | 0 | 0 | 1 (9.1%) | 0 | 1 (4.2%) |
| Orchitis noninfective | 0 | 0 | 0 | 0 | 1 (9.1%) | 0 | 1 (4.2%) |
| Osteorrhagia | 0 | 0 | 0 | 0 | 0 | 1 (20.0%) | 1 (4.2%) |
| Pain | 0 | 0 | 0 | 0 | 0 | 1 (20.0%) | 1 (4.2%) |
| Paraesthesia | 0 | 0 | 0 | 0 | 1 (9.1%) | 0 | 1 (4.2%) |
| Periorbital oedema | 0 | 0 | 0 | 0 | 1 (9.1%) | 0 | 1 (4.2%) |
| Photophobia | 0 | 0 | 0 | 0 | 1 (9.1%) | 0 | 1 (4.2%) |
| Pneumococcal sepsis | 0 | 0 | 0 | 0 | 1 (9.1%) | 0 | 1 (4.2%) |
| Pneumonia fungal | 0 | 0 | 0 | 0 | 0 | 1 (20.0%) | 1 (4.2%) |
| Pruritus | 0 | 0 | 1 (33.3%) | 0 | 0 | 0 | 1 (4.2%) |
| Rash | 0 | 0 | 0 | 0 | 0 | 1 (20.0%) | 1 (4.2%) |
| Rash maculo-papular | 0 | 0 | 0 | 0 | 1 (9.1%) | 0 | 1 (4.2%) |
| Respiratory syncytial virus | 0 | 0 | 0 | 0 | 0 | 1 (20.0%) | 1 (4.2%) |
| infection | | | | | | | |
| Restlessness | 0 | 1 (100%) | 0 | 0 | 0 | 0 | 1 (4.2%) |
| Rhinitis | 0 | 0 | 0 | 1 (33.3%) | 0 | 0 | 1 (4.2%) |
| Rhinovirus infection | 0 | 0 | 0 | 0 | 1 (9.1%) | 0 | 1 (4.2%) |
| Rotavirus infection | 0 | 0 | 0 | 0 | 1 (9.1%) | 0 | 1 (4.2%) |
| Scapula fracture | 0 | 1 (100%) | 0 | 0 | 0 | 0 | 1 (4.2%) |
| Sinus bradycardia | 0 | 0 | 1 (33.3%) | 0 | 0 | 0 | 1 (4.2%) |
| Squamous cell carcinoma of | 0 | 0 | 0 | 0 | 1 (9.1%) | 0 | 1 (4.2%) |
| skin | | | | | | | |
| Subcutaneous abscess | 0 | 0 | 0 | 0 | 0 | 1 (20.0%) | 1 (4.2%) |
| Testicular pain | 0 | 0 | 0 | 0 | 1 (9.1%) | 0 | 1 (4.2%) |
| Tinnitus | 0 | 0 | 0 | 0 | 1 (9.1%) | 0 | 1 (4.2%) |
| Upper respiratory tract infection | 0 | 0 | 0 | 0 | 0 | 1 (20.0%) | 1 (4.2%) |
| Vascular device infection | 0 | 0 | 0 | 0 | 0 | 1 (20.0%) | 1 (4.2%) |
| Vitreous floaters | 0 | 0 | 0 | 0 | 1 (9.1%) | 0 | 1 (4.2%) |
| Weight decreased | 1 (100%) | 0 | 0 | 0 | 0 | 0 | 1 (4.2%) |

Percentages are based on N, Multiple adverse events with a same preferred term are only counted once for each subject.
Coded with MedDRA version 25.0.
Treatment Emergent Adverse Events are AEs that started or pre-existing AEs that worsened on/after the first IMP and within 30 days of last IMP.

TABLE 10
Related Treatment-Emergent Adverse Events

| | 0.2/0.6 mg/kg | 2 mg/kg | 4 mg/kg | 8 mg/kg | 16 mg/kg | 24 mg/kg | Total |
|---|---|---|---|---|---|---|---|
| | (N = 1) | (N = 1) | (N = 3) | (N = 3) | (N = 11) | (N = 5) | (N = 24) |
| Number of subjects with at | 0 | 0 | 3 (100%) | 3 (100%) | 11 (100%) | 4 (80.0%) | 21 (87.5%) |
| least one related TEAE, n(%) | | | | | | | |
| Infusion related reaction | 0 | 0 | 2 (66.7%) | 3 (100%) | 9 (81.8%) | 4 (80.0%) | 18 (75.0%) |
| Neutropenia | 0 | 0 | 1 (33.3%) | 2 (66.7%) | 6 (54.5%) | 3 (60.0%) | 12 (50.0%) |
| Thrombocytopenia | 0 | 0 | 0 | 0 | 2 (18.2%) | 2 (40.0%) | 4 (16.7%) |
| Anaemia | 0 | 0 | 0 | 0 | 1 (9.1%) | 2 (40.0%) | 3 (12.5%) |
| Conjunctival oedema | 0 | 0 | 0 | 1 (33.3%) | 1 (9.1%) | 1 (20.0%) | 3 (12.5%) |
| Diarrhoea | 0 | 0 | 1 (33.3%) | 0 | 2 (18.2%) | 0 | 3 (12.5%) |
| Fatigue | 0 | 0 | 0 | 0 | 2 (18.2%) | 1 (20.0%) | 3 (12.5%) |
| Fibrin D dimer increased | 0 | 0 | 0 | 3 (100%) | 0 | 0 | 3 (12.5%) |
| Hypophosphataemia | 0 | 0 | 0 | 0 | 1 (9.1%) | 2 (40.0%) | 3 (12.5%) |
| Leukopenia | 0 | 0 | 0 | 0 | 2 (18.2%) | 0 | 2 (8.3%) |
| Alanine aminotransferase increased | 0 | 0 | 1 (33.3%) | 10 | 0 | 0 | 1 (4.2%) |
| Appendicitis | 0 | 0 | 0 | 0 | 0 | 1 (20.0%) | 1 (4.2%) |
| Bacteraemia | 0 | 0 | 0 | 0 | 1 (9.1%) | 0 | 1 (4.2%) |
| Blood phosphorus decreased | 0 | 0 | 0 | 0 | 1 (9.1%) | 0 | 1 (4.2%) |
| Cough | 0 | 0 | 0 | 0 | 1 (9.1%) | 0 | 1 (4.2%) |
| Febrile neutropenia | 0 | 0 | 0 | 0 | 0 | 1 (20.0%) | 1 (4.2%) |
| Gingival pain | 0 | 0 | 0 | 0 | 1 (9.1%) | 0 | 1 (4.2%) |
| Headache | 0 | 0 | 0 | 0 | 0 | 1 (20.0%) | 1 (4.2%) |
| Metapneumovirus infection | 0 | 0 | 0 | 0 | 1 (9.1%) | 0 | 1 (4.2%) |
| Neutropenic sepsis | 0 | 0 | 0 | 0 | 0 | 1 (20.0%) | 1 (4.2%) |
| Pneumonia fungal | 0 | 0 | 0 | 0 | 0 | 1 (20.0%) | 1 (4.2%) |
| Rotavirus infection | 0 | 0 | 0 | 0 | 1 (9.1%) | 0 | 1 (4.2%) |
| Syncope | 0 | 0 | 0 | 0 | 1 (9.1%) | 0 | 1 (4.2%) |
| Vision blurred | 0 | 0 | 0 | 0 | 1 (9.1%) | 0 | 1 (4.2%) |
| Vitreous floaters | 0 | 0 | 0 | 0 | 1 (9.1%) | 0 | 1 (4.2%) |
| Vomiting | 0 | 0 | 0 | 0 | 1 (9.1%) | 0 | 1 (4.2%) |

Percentages are based on N, Multiple adverse events with a same preferred term are only counted once for each subject.
Related as assessed by the investigator.
Coded with MedDRA version 25.0.
Treatment Emergent Adverse Events are AEs that started or pre-existing AEs that worsened on/after the first IMP and within 30 days of last IMP.

TABLE 11
Serious Treatment-Emergent Adverse Events

| | 0.2/0.6 mg/kg | 2 mg/kg | 4 mg/kg | 8 mg/kg | 16 mg/kg | 24 mg/kg | Total |
|---|---|---|---|---|---|---|---|
| | (N = 1) | (N = 1) | (N = 3) | (N = 3) | (N = 11) | (N = 5) | (N = 24) |
| Number of subjects with | 1 (100%) | 1 (100%) | 1 (33.3%) | 2 (66.7%) | 6 (54.5%) | 4 (80.0%) | 15 (62.5%) |
| at least one STEAE, n(%) | | | | | | | |
| COVID-19 | 0 | 0 | 0 | 1 (33.3%) | 1 (9.1%) | 1 (20.0%) | 3 (12.5%) |
| Pyrexia | 1 (100%) | 0 | 1 (33.3%) | 1 (33.3%) | 0 | 0 | 3 (12.5%) |
| Infusion related reaction | 0 | 0 | 1 (33.3%) | 0 | 1 (9.1%) | 0 | 2 (8.3%) |
| Pneumonia | 0 | 1 (100%) | 0 | 0 | 1 (9.1%) | 0 | 2 (8.3%) |
| Acute myocardial infarction | 0 | 0 | 0 | 0 | 1 (9.1%) | 0 | 1 (4.2%) |
| Adenoviral upper respiratory | 0 | 0 | 0 | 0 | 0 | 1 (20.0%) | 1 (4.2%) |
| infection | | | | | | | |
| Appendicitis | 0 | 0 | 0 | 0 | 0 | 1 (20.0%) | 1 (4.2%) |
| Dyspnoea | 0 | 0 | 0 | 0 | 1 (9.1%) | 0 | 1 (4.2%) |
| Febrile neutropenia | 0 | 0 | 0 | 0 | 0 | 1 (20.0%) | 1 (4.2%) |
| Fibrin D dimer increased | 0 | 0 | 0 | 1 (33.3%) | 0 | 0 | 1 (4.2%) |
| Hypervolaemia | 0 | 0 | 0 | 0 | 1 (9.1%) | 0 | 1 (4.2%) |
| Neutropenic sepsis | 0 | 0 | 0 | 0 | 0 | 1 (20.0%) | 1 (4.2%) |
| Pneumococcal sepsis | 0 | 0 | 0 | 0 | 1 (9.1%) | 0 | 1 (4.2%) |
| Pneumonia fungal | 0 | 0 | 0 | 0 | 0 | 1 (20.0%) | 1 (4.2%) |
| Respiratory syncytial virus infection | 0 | 0 | 0 | 0 | 0 | 1 (20.0%) | 1 (4.2%) |
| Rhinovirus infection | 0 | 0 | 0 | 0 | 1 (9.1%) | 0 | 1 (4.2%) |
| Sinus bradycardia | 0 | 0 | 1 (33.3%) | 0 | 0 | 0 | 1 (4.2%) |
| Urinary tract infection | 0 | 0 | 0 | 0 | 0 | 1 (20.0%) | 1 (4.2%) |
| Vascular device infection | 0 | 0 | 0 | 0 | 0 | 1 (20.0%) | 1 (4.2%) |

Percentages are based on N, Multiple adverse events with a same preferred term are only counted once for each subject.
Coded with MedDRA version 25.0.
Serious Treatment Emergent Adverse Events are serious AEs that started or pre-existing AEs that became serious on/after the first IMP and within 30 days of last IMP.

TABLE 12
Related Serious Treatment-Emergent Adverse Events

| | 0.2/0.6 mg/kg | 2 mg/kg | 4 mg/kg | 8 mg/kg | 16 mg/kg | 24 mg/kg | Total |
|---|---|---|---|---|---|---|---|
| | (N = 1) | (N = 1) | (N = 3) | (N = 3) | (N = 11) | (N = 5) | (N = 24) |
| Number of subjects with at least | 0 | 0 | 1 (33.3%) | 1 (33.3%) | 1 (9.1%) | 1 (20.0%) | 4 (16.7%) |
| one related STEAE, n(%) | | | | | | | |
| Infusion related reaction | 0 | 0 | 1 (33.3%) | 0 | 1 (9.1%) | 0 | 2 (8.3%) |
| Appendicitis | 0 | 0 | 0 | 0 | 0 | 1 (20.0%) | 1 (4.2%) |
| Fibrin D dimer increased | 0 | 0 | 0 | 1 (33.3%) | 0 | 0 | 1 (4.2%) |
| Neutropenic sepsis | 0 | 0 | 0 | 0 | 0 | 1 (20.0%) | 1 (4.2%) |
| Pneumonia fungal | 0 | 0 | 0 | 0 | 0 | 1 (20.0%) | 1 (4.2%) |

Percentages are based on N, Multiple adverse events with a same preferred term are only counted once for each subject.
Related as assessed by the investigator.
Coded with MedDRA version 25.0.
Serious Treatment Emergent Adverse Events are serious AEs that started or pre-existing AEs that became serious on/after the first IMP and within 30 days of last IMP.

TABLE 13
Summary of Infusion-Related Reaction by Dose Level

| | 0.2/0.6 mg/kg | 2 mg/kg | 4 mg/kg | 8 mg/kg | 16 mg/kg | 24 mg/kg | Total |
|---|---|---|---|---|---|---|---|
| | (N = 1) | (N = 1) | (N = 3) | (N = 3) | (N = 11) | (N = 5) | (N = 24) |
| Number of subjects with | | | | | | | |
| at least one, n(%) | | | | | | | |
| AESI: IRR | 0 | 0 | 2 (66.7%) | 3 (100%) | 9 (81.8%) | 4 (80.0%) | 18 (75.0%) |
| Grade 1 | 0 | 0 | 0 | 0 | 2 (18.2%) | 0 | 2 (8.3%) |
| Grade 2 | 0 | 0 | 0 | 3 (100%) | 7 (63.6%) | 2 (40.0%) | 12 (50.0%) |
| Grade 3 | 0 | 0 | 2 (66.7%) | 0 | 0 | 2 (40.0%) | 4 (16.7%) |
| Number of subjects with | | | | | | | |
| at least one, n(%) | | | | | | | |
| IRR leading to permanent | 0 | 0 | 0 | 0 | 2 (18.2%) | 0 | 2 (8.3%) |
| treatment discontinuation | | | | | | | |
| IRR leading to dose interruption | 0 | 0 | 2 (66.7%) | 2 (66.7%) | 7 (63.6%) | 4 (80.0%) | 15 (62.5%) |

Infusion-related reactions are limited to events flagged as AESI and with a preferred term of Infusion related reaction.

### Example 5—Evaluation of Lymphocyte Populations in Whole Blood of Patients Dosed with IgG1-C-E430G

### Methods

NK and T cell populations were evaluated in whole blood from RRMM patients dosed with 0.2/0.6 to 24 mg/kg IgG1-C-E430G in the dose escalation part of the first-in-human trial of IgG1-C-E430G.

As of the data cutoff date of 3 Oct. 2022, preliminary pharmacodynamic data were available from 24 subjects with RRMM dosed with IgG1-C-E430G in the Dose Escalation part (0.2/0.6 to 24 mg/kg) of the first-in-human trial.

As of the data cutoff date of 14 Aug. 2023, preliminary pharmacodynamic data were available from 24 subjects with RRMM dosed with IgG1-C-E430G in the Dose Escalation part (0.2/0.6 to 24 mg/kg) of the first-in-human trial.

Blood samples for immunophenotyping (IPT) were drawn from patients according to the scheme shown in Table 14. Blood was collected into 5 mL EDTA blood collection tubes and stored ambient.

TABLE 14
Schedule for blood sampling.

| Cycle 1 | Timepoint |
|---|---|
| Day 1 | Predose (−30 min) |
| Day 2 | Predose (−30 min) Day 2 |
| Day 3 | End of Day 2 infusion +24 h (±2 h) |
| Day 5 | End of Day 2 infusion +72 h (±24 h) |
| Day 8 | Predose (−30 min) |
| Day 9 | Predose (−30 min)* |
| Day 10 | End of Day 9 infusion +24 h (±2 h)* |
| Day 15 | Predose (−30 min) |
| Day 22 | Predose (−30 min) |

*Dose level 1 (0.2/0.6 mg/kg only)

For each blood sample 50 μL of whole blood was added in a polystyrene tube, and 78.75 μL of antibody cocktail (Table 15) was added to each tube, after which tubes were mixed by vortexing gently. After incubation in the dark at room temperature for 15 minutes, 450 μL 1×FACS Lyse buffer (BD Bioscience, USA; Cat #349202) was added and incubated in the dark at room temperature for 15 minutes and subsequently analyzed on a FACSCanto flow cytometer (BD Biosciences, USA). The various lymphocyte subpopulations were identified as follows:
NK cells: CD3−/CD56+/CD16+
T cells: CD3+
Monocytes: CD14+
B cells: CD3−/CD19+
NKT cells: CD3+/CD56+/CD16+

TABLE 15
Components of the used antibody cocktail.

| Material | Company | Catalog number | Volume per sample | |
|---|---|---|---|---|
| Brillant Stain Buffer | BD Bioscience | 563794 | 50 | μL |
| CD19 BV421 (HIB19) | BD Bioscience | 562440 | 2.5 | μL |
| CD4 BV510 (SK3) | BD Bioscience | 562971 | 2.5 | μL |
| CD3 FITC (SK7) | BD Bioscience | 345764 | 1.25 | μL |
| CD16 PE (B73.1) | BD Bioscience | 332779 | 2.5 | μL |
| CD56 PE (NCAM16.2) | BD Bioscience | 345812 | 2.5 | μL |
| CD20 PE-Cy7 (L27) | BD Bioscience | 335828 | 2.5 | μL |
| CD45 PerCP-Cy5.5 (2D1) | BD Bioscience | 332784 | 10 | μL |
| CD8 APC (SK1) | BD Bioscience | 345775 | 2.5 | μL |
| CD14 APC-H7 (MφP9) | BD Bioscience | 641394 | 2.5 | μL |

Results: NK Cell Numbers in Peripheral Blood of Patients Dosed with IgG1-C-E430G

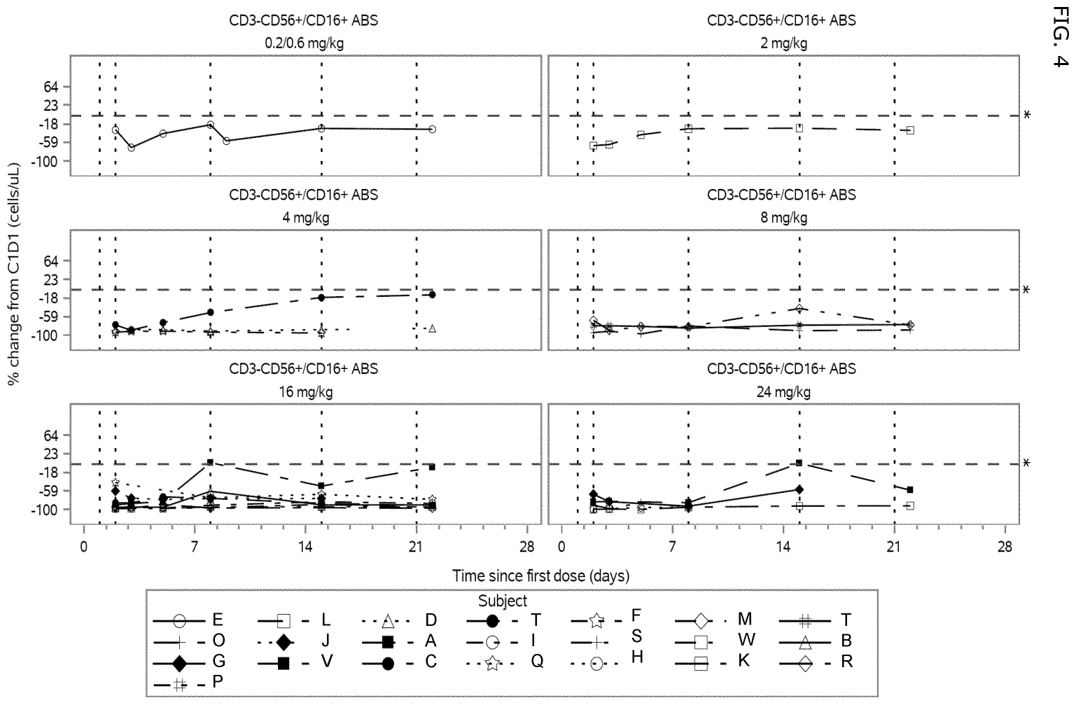

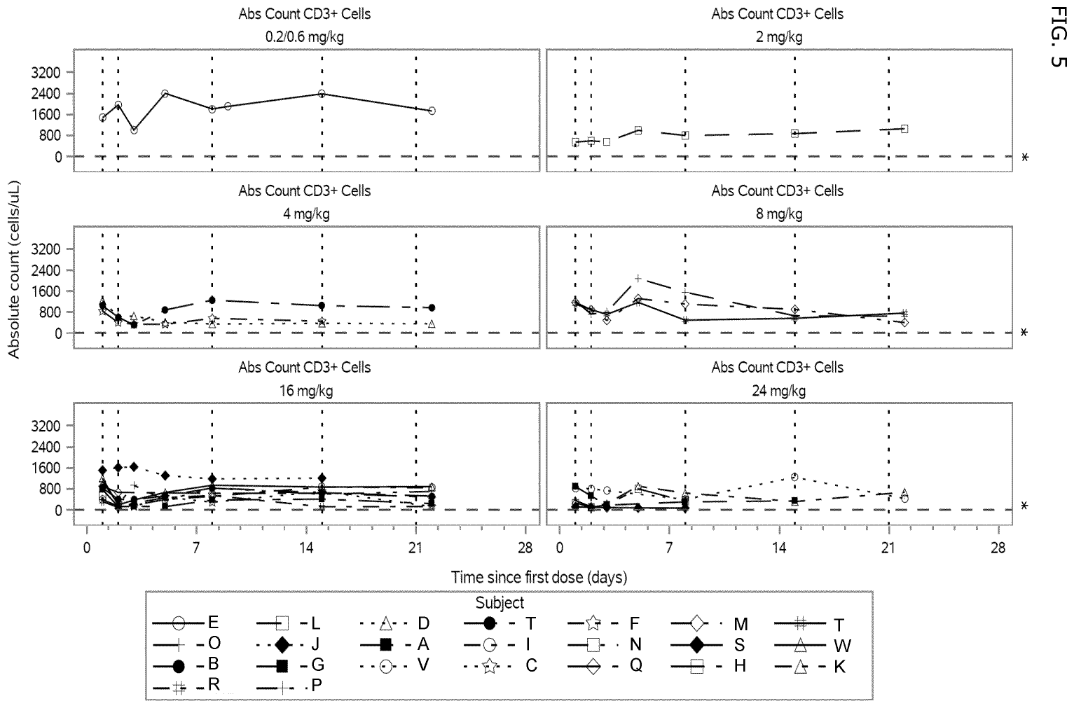

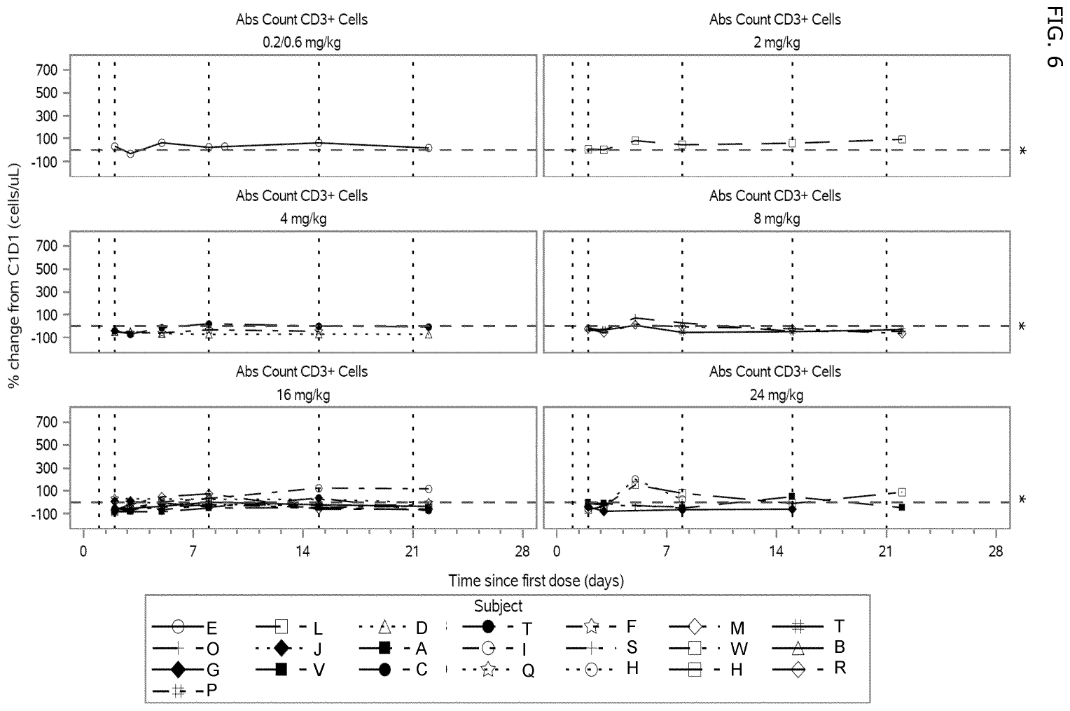

FIG. 3 shows that IgG1-C-E430G administration was associated with a rapid decrease in the number of peripheral blood NK cells (CD3−/CD56+/CD16+ cells) at all evaluated dose levels in all subjects. The median maximum percentage reduction in the number of NK cells, compared to baseline (C1D1, pre-dose) was 97% (range 66%-100%, n=21) in evaluable patients. Patients with BL value below LLOQ, no BL value or only a BL value were excluded from this analysis (FIG. 4). NK cell numbers remained low while on IgG1-C-E430G treatment in majority of patients. Baseline and maximum changes in NK cell numbers and percentages in peripheral blood of patients dosed with IgG1-C-E430G are summarized in Table 16.

In conclusion, prominent NK cell reduction was observed in patients dosed with IgG1-C-E430G at all evaluated dose levels. The observed NK cell decrease confirms biological activity of IgG1-C-E430G in patients, and is indicative of the ADCC activity of IgG1-C-E430G.

TABLE 16
NK Cell population in Peripheral Blood in patients dosed with IgG1-C-E430G

| CD3−/ | | | | | | | |
|---|---|---|---|---|---|---|---|
| CD56+/ | Dose Level | | | | | | |
| CD16+ ABS | 0.2/0.6 mg/kg | 2 mg/kg | 4 mg/kg | 8 mg/kg | 16 mg/kg | 24 mg/kg | Total |
| (Cells/uL) | (N = 1) | (N = 1) | (N = 3) | (N = 3) | (N = 11) | (N = 5) | (N = 24) |
| Baseline | | | | | | | |
| N | 1 | 1 | 3 | 3 | 11 | 5 | 24 |
| Mean | 149.0 | 83.0 | 182.0 | 77.3 | 219.4 | 246.8 | 194.0 |
| Std Dev | N/E | N/E | 271.17 | 7.51 | 198.19 | 171.06 | 178.60 |
| Median | 149.0 | 83.0 | 33.0 | 77.0 | 157.0 | 235.0 | 117.5 |
| Min, Max | 149, 149 | 83, 83 | 18, 495 | 70, 85 | 20, 632 | 54, 446 | 18, 632 |
| Maximum | | | | | | | |
| change | | | | | | | |
| N | 1 | 1 | 3 | 3 | 9 | 5 | 22 |
| Mean | −105.0 | −55.0 | −173.3 | −72.0 | −169.4 | −202.0 | −156.0 |
| Std Dev | N/E | N/E | 258.78 | 4.00 | 182.98 | 224.96 | 176.11 |
| Median | −105.0 | −55.0 | −32.0 | −72.0 | −124.0 | −218.0 | −87.5 |
| Min, Max | −105, −105 | −55, −55 | −472, −16 | −76, −68 | −617, −20 | −442, 93 | −617, 93 |
| Maximum | | | | | | | |
| percentage | | | | | | | |
| change | | | | | | | |
| N | 1 | 1 | 3 | 3 | 9 | 5 | 22 |
| Mean | −70.4698 | −66.2651 | −93.7374 | −93.3537 | −94.8450 | −60.7301 | −84.3302 |
| Std Dev | N/E | N/E | 4.27596 | 3.86781 | 4.48636 | 82.13303 | 39.04756 |
| Median | −70.4698 | −66.2651 | −95.3535 | −93.5065 | −97.4522 | −98.1481 | −95.4545 |
| Min, Max | −70.470, −70.470 | −66.265, −66.265 | −96.970, −88.889 | −97.143, −89.412 | −100.000, −88.439 | −99.744, 86.111 | −100.000, 86.111 |

Cutoff date: 3 Oct. 2022

Results: T Cell Numbers in Peripheral Blood of Patients Dosed with IgG1-C-E430G

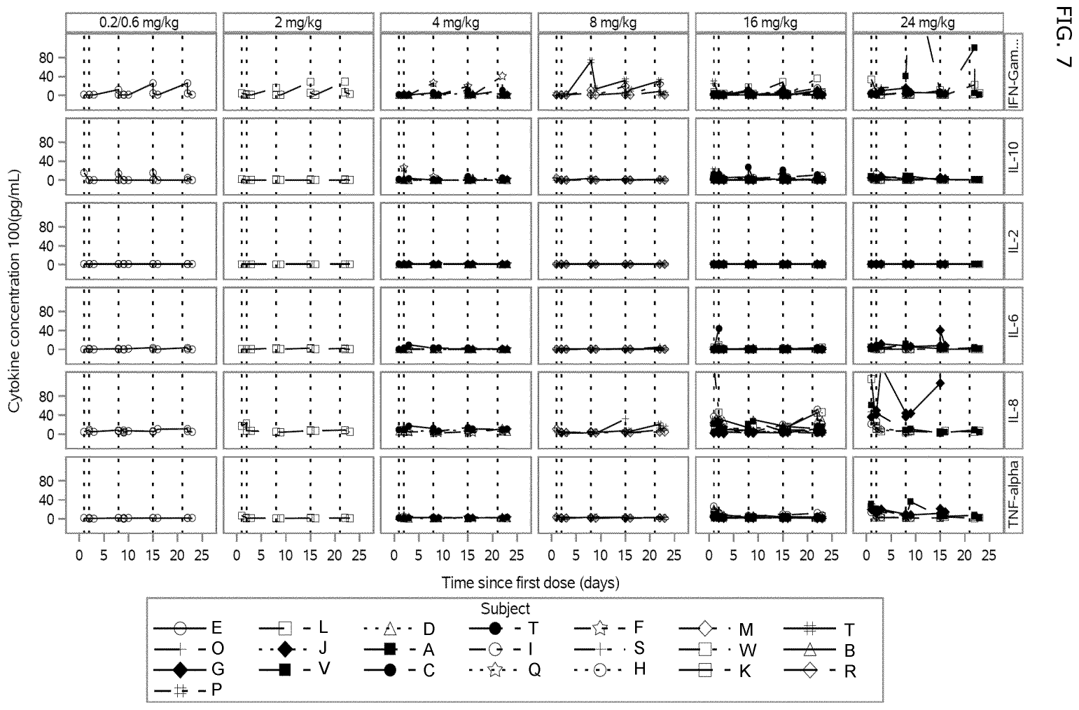

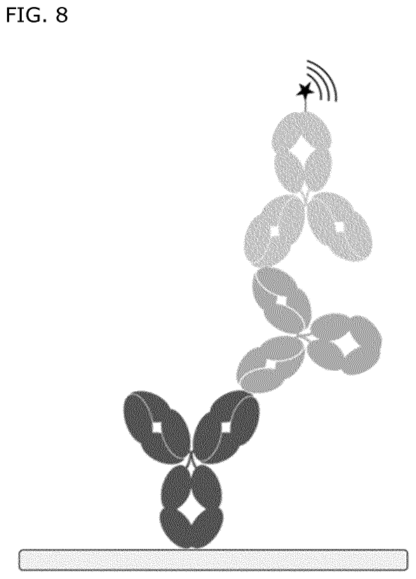

FIG. 5 shows a transient decrease in T cells (CD3+ cells) after administration of the first dose of IgG1-C-E430G at dose levels ≥4 mg/kg (data cut off: 3 Oct. 2022). A subsequent increase in the number of peripheral blood T cells (≥100% increase from baseline) was observed in 7 out 16 patients across 5 out of 6 dose levels, particularly in patients that had not received prior treatment with CD38 mAb (FIG. 6). Baseline and maximum changes in T cell numbers and percentages in peripheral blood of patients dosed with IgG1-C-E430G are summarized in Table 17.

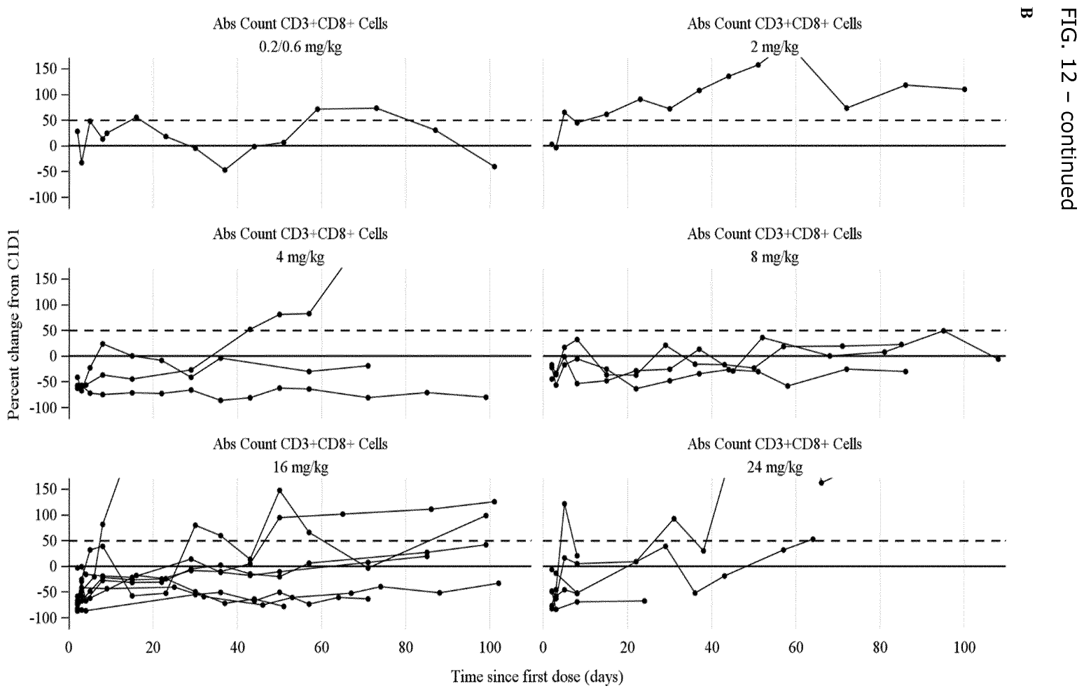

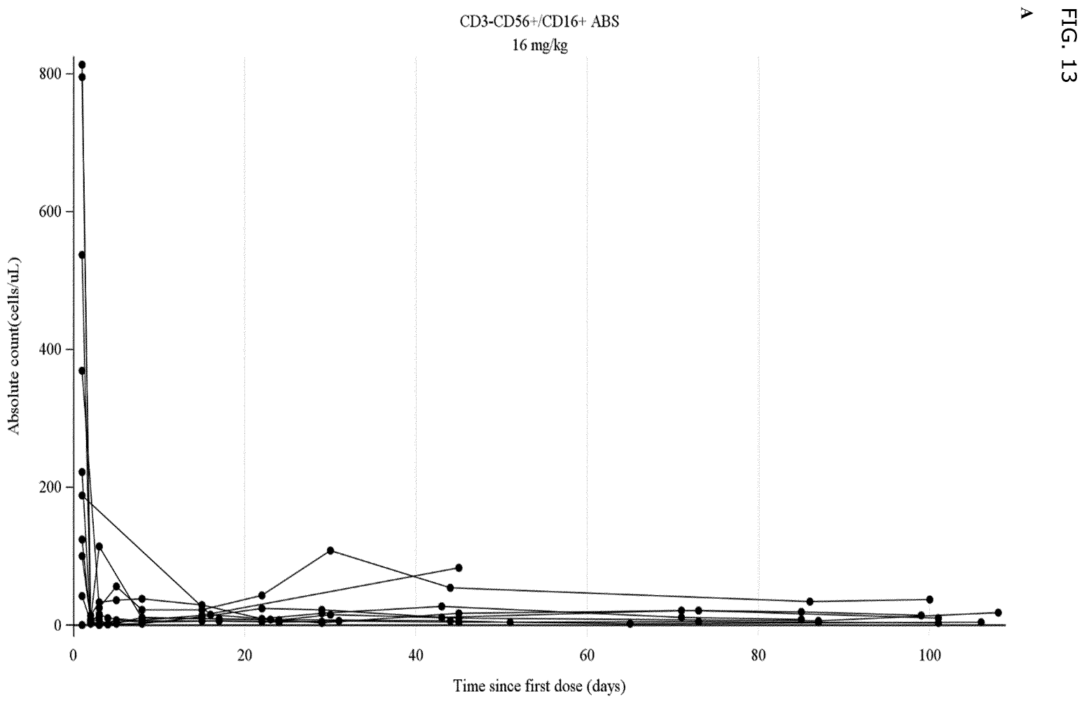

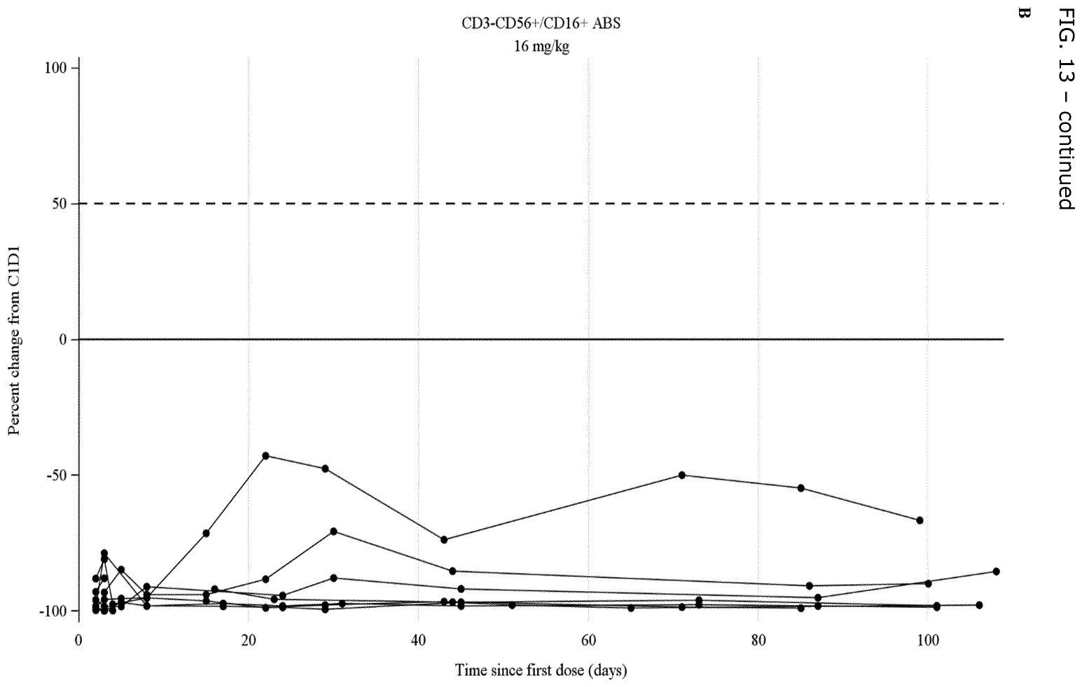

FIG. 11 and FIG. 12 shows a transient decrease in CD3+CD4+ and CD3+CD8+ T cells after administration of the first dose of IgG1-C-E430G at dose levels ≥4 mg/kg (data cut off: 14 Aug. 2023). A subsequent increase in the number of peripheral blood CD3+CD4+ T cells (>50% increase from baseline for 2 visits) was observed in 6 out 21 evaluable patients across 5 out of 6 dose levels, particularly in patients that had not received prior treatment with CD38 mAb (FIG. 11). A subsequent increase in the number of peripheral blood CD3+D8+ T cells (>50% increase from baseline for ≥2 visits) was observed in 8 out 21 evaluable patients across 5 out of 6 dose levels, particularly in patients that had not received prior treatment with CD38 mAb (FIG. 12). Baseline and maximum changes in CD3+/CD4+ and CD3+/CD8+ T cell numbers and percentages in peripheral blood of patients dosed with IgG1-C-E430G are summarized in Table 17 and Table 18.

In conclusion, increases in peripheral T cell numbers were observed in a subset of patients dosed with IgG1-C-E430G across dose levels. The observed T cell expansion confirms biological activity of IgG1-C-E430G in vivo, and is indicative of the immunomodulatory activity of IgG1-C-E430G in patients.

TABLE 17
T Cells in Peripheral Blood of patients dosed with IgG1-C-E430G

| Abs Count | Dose Level | | | | | | |
|---|---|---|---|---|---|---|---|
| CD3+ Cells | 0.2/0.6 mg/kg | 2 mg/kg | 4 mg/kg | 8 mg/kg | 16 mg/kg | 24 mg/kg | Total |
| (Cells/uL) | (N = 1) | (N = 1) | (N = 3) | (N = 3) | (N = 11) | (N = 5) | (N = 24) |
| Baseline | | | | | | | |
| N | 1 | 1 | 3 | 3 | 11 | 5 | 24 |
| Mean | 1469.0 | 546.0 | 1039.0 | 1165.7 | 751.3 | 495.6 | 807.1 |
| Std Dev | N/E | N/E | 201.52 | 57.35 | 374.67 | 348.16 | 392.83 |
| Median | 1469.0 | 546.0 | 1042.0 | 1173.0 | 555.0 | 361.0 | 837.0 |
| Min, Max | 1469, 1469 | 546, 546 | 836, 1239 | 1105, 1219 | 318, 1508 | 119, 891 | 119, 1508 |
| Maximum | | | | | | | |
| change | | | | | | | |
| N | 1 | 1 | 3 | 3 | 9 | 5 | 22 |
| Mean | 1910.0 | 1297.0 | −2.7 | 716.0 | 970.6 | 178.2 | 680.6 |
| Std Dev | N/E | N/E | 1515.95 | 1258.95 | 1771.15 | 897.20 | 1404.01 |
| Median | 1910.0 | 1297.0 | −731.0 | 863.0 | 524.0 | −48.0 | 530.0 |
| Min, Max | 1910, 1910 | 1297, 1297 | −1017, 1740 | −610, 1895 | −653, 4959 | −709, 1548 | −1017, 4959 |
| Maximum | | | | | | | |
| percentage | | | | | | | |
| change | | | | | | | |
| N | 1 | 1 | 3 | 3 | 9 | 5 | 22 |
| Mean | 130.0204 | 237.5458 | 18.6327 | 59.0479 | 106.9540 | 91.2254 | 91.7873 |
| Std Dev | N/E | N/E | 164.22126 | 108.85409 | 171.75380 | 219.45144 | 161.88119 |
| Median | 130.0204 | 237.5458 | −70.1536 | 70.7957 | 103.5000 | −40.3361 | 87.1479 |
| Min, Max | 130.020, 130.020 | 237.546, 237.546 | −82.082, 208.134 | −55.204, 161.552 | −84.041, 373.077 | −79.574, 428.809 | −84.041, 428.809 |

Cutoff date: 3 Oct. 2022

TABLE 18
CD4+ T Cells in Peripheral Blood of patients dosed with IgG1-C-E430G (dose escalation part, data cut off: 14 Aug. 2023)

| Abs Count | Dose level | | | | | | |
|---|---|---|---|---|---|---|---|
| CD3+CD4+ Cells | 0.2/0.6 mg/kg | 2 mg/kg | 4 mg/kg | 8 mg/kg | 16 mg/kg | 24 mg/kg | Total |
| (Cells/uL) | (N = 1) | (N = 1) | (N = 3) | (N = 3) | (N = 11) | (N = 5) | (N = 24) |
| Baseline | | | | | | | |
| N | 1 | 1 | 3 | 3 | 9 | 5 | 22 |
| Mean | 104 | 168 | 316.3 | 381 | 269.7 | 183.8 | 259.5 |
| Std Dev | N/E | N/E | 89.79 | 143.07 | 140.69 | 75.75 | 129.92 |
| Median | 104 | 168 | 308 | 353 | 218 | 200 | 224.5 |
| Min, Max | 104.0, 104.0 | 168.0, 168.0 | 231.0, 410.0 | 254.0, 536.0 | 112.0, 567.0 | 63.0, 268.0 | 63.0, 567.0 |
| Maximum | | | | | | | |
| change | | | | | | | |
| N | 1 | 1 | 3 | 3 | 8 | 5 | 21 |
| Mean | 270 | 354 | −17.3 | 305 | −34.9 | 176.6 | 99.6 |
| Std Dev | N/E | N/E | 316.84 | 434.33 | 270.25 | 298.55 | 306.72 |
| Median | 270 | 354 | −183 | 336 | −87 | 146 | 69 |
| Min, Max | 270.0, 270.0 | 354.0, 354.0 | −217.0, 348.0 | −144.0, 723.0 | −451.0, 401.0 | −189.0, 544.0 | −451.0, 723.0 |
| Maximum | | | | | | | |
| percentage | | | | | | | |
| change | | | | | | | |
| N | 1 | 1 | 3 | 3 | 8 | 5 | 21 |
| Mean | 259.6 | 210.7 | −21.6 | 57.8 | −6.5 | 91.8 | 47 |
| Std Dev | N/E | N/E | 92.32 | 101.12 | 90.93 | 146.71 | 122.65 |
| Median | 259.6 | 210.7 | −70.5 | 95.2 | −57.4 | 73 | 31.7 |
| Min, Max | 259.6, 259.6 | 210.7, 210.7 | −79.2, 84.9 | −56.7, 134.9 | −79.5, 147.6 | −70.5, 254.2 | −79.5, 259.6 |

Cutoff date: 14 Aug. 2023

TABLE 19
CD8+ T Cells in Peripheral Blood of patients dosed with IgG1-C-E430G (dose escalation part, data cut off: 14 Aug. 2023)

| Abs Count | Dose level | | | | | | |
|---|---|---|---|---|---|---|---|
| CD3+CD8+ Cells | 0.2/0.6 mg/kg | 2 mg/kg | 4 mg/kg | 8 mg/kg | 16 mg/kg | 24 mg/kg | Total |
| (Cells/uL) | (N = 1) | (N = 1) | (N = 3) | (N = 3) | (N = 11) | (N = 5) | (N = 24) |
| Baseline | | | | | | | |
| n | 1 | 1 | 3 | 3 | 9 | 5 | 22 |
| Mean | 1377 | 375 | 725.7 | 714.3 | 545 | 322.2 | 572.2 |
| Std Dev | N/E | N/E | 269.69 | 55.08 | 316.22 | 300.46 | 341.54 |
| Median | 1377 | 375 | 777 | 697 | 530 | 152 | 587.5 |
| Min, Max | 1377.0, 1377.0 | 375.0, 375.0 | 434.0, 966.0 | 670.0, 776.0 | 159.0, 1058.0 | 55.0, 663.0 | 55.0, 1377.0 |
| Maximum | | | | | | | |
| change | | | | | | | |
| n | 1 | 1 | 3 | 3 | 8 | 5 | 21 |
| Mean | 1012 | 935 | −100.3 | −79 | 226.3 | 171.4 | 194.1 |
| Std Dev | N/E | N/E | 1009.69 | 508.22 | 738.58 | 558.65 | 683.19 |
| Median | 1012 | 935 | −523 | −298 | 43 | 129 | 129 |
| Min, Max | 1012.0, 1012.0 | 935.0, 935.0 | −830.0, 1052.0 | −441.0, 502.0 | −497.0, 1287.0 | −554.0, 979.0 | −830.0, 1287.0 |
| Maximum | | | | | | | |
| percentage | | | | | | | |
| change | | | | | | | |
| n | 1 | 1 | 3 | 3 | 8 | 5 | 21 |
| Mean | 73.5 | 249.3 | 29.7 | −14.4 | 96 | 135.4 | 86.4 |
| Std Dev | N/E | N/E | 184.42 | 69.1 | 231.74 | 296.31 | 210.26 |
| Median | 73.5 | 249.3 | −67.3 | −44.5 | 40.9 | 52.8 | 52.8 |
| Min, Max | 73.5, 73.5 | 249.3, 249.3 | −85.9, 242.4 | −63.3, 64.7 | −86.8, 604.2 | −83.6, 644.1 | −86.8, 644.1 |

Cutoff date: 14 Aug. 2023

Results: Monocyte, B Cell, and NKT Cell Numbers in Peripheral Blood of Patients Dosed with IgG1-C-E430G

Monocyte, B cell and NKT-like cell levels fluctuated in peripheral blood of patients post IgG1-C-E430G dosing, but no consistent or dose-dependent decreases or increases of these lymphocyte populations were observed in patients dosed with IgG1-C-E430G (data not shown).

In conclusion, the enhanced CDC activity of IgG1-C-E430G is not associated with reductions in monocytes, B cells, or NKT-like cells in response to treatment with IgG1-C-E430G in the clinical setting.

### Example 6—Evaluation of Complement Component C2 Levels in Plasma of Patients Dosed with IgG1-C-E430G

### Methods

Blood sampling for analysis of complement C2 in plasma was performed according to the scheme shown in Table 20. Plasma from a lavender top (EDTA) tube was collected. Tubes were mixed thoroughly and centrifuged at room temp within one half hour of collection. The cell-free plasma was transferred to a clean tube and immediately frozen on dry ice or at −70° C. until further use. C2 levels were measured using a Radial Immunodiffusion (RID) assay at Quest Diagnostics Nichols Institute (Secaucus, NJ).

In brief, test samples were mixed gently immediately before use and applied on the wells of RID plates, which contain monospecific antibody to C2 in agarose gel. After sample application, plates were tightly closed with a lid and the plate stored flat at room temperature (approximately 20-24° C.) for 18-120 hours. To minimize evaporation, plates were sealed in foil or stored in a sealed plastic box containing damp tissue paper during incubation. Final ring diameters were measured to the nearest 0.1 mm using a jewellers' eyepiece or digital RID plate reader. C2 concentrations in each test sample were read directly from the RID reference table of were determined by comparison to a reference curve.

TABLE 20
Schedule for blood sampling for complement analyses.

| Cycle 1 | Timepoint |
|---|---|
| Day 1 | Predose (−30 min) |
| | End of infusion +4 h (±30 min) |
| Day 2 | Predose (−30 min) Day 2 |
| | End of Day 2 infusion +4 h (±30 min) |
| Day 3 | End of Day 2 infusion +24 h (±2 h) |
| Day 8 | Predose (−30 min) |
| | End of infusion +4 h (±30 min) |
| Day 9 | Predose (−30 min)* |
| | End of infusion +4 h (±30 min)* |
| | End of Day 8 infusion +24 h (±2 h) ** |
| Day 10 | End of Day 9 infusion +24 h (±2 h)* |
| Day 15 | Predose (−30 min) |
| | End of infusion +4 h (±30 min) |
| Day 16 | End of Day 15 infusion +24 h (±2 h) |
| Day 22 | Predose (−30 min) |
| | End of infusion +4 h (±30 min) |
| Day 23 | End of Day 22 infusion +24 h (±2 h) |

*Dose level 1 (0.2/0.6 mg/kg)
**All cohorts except Dose level 1 (0.2/0.6 mg/kg)

Results: Complement Component C2 Levels in Plasma of Patients Dosed with IgG1-C-E430G

Table 21 shows transient reductions in complement component C2 at all doses, with a median peak reduction from baseline (C1 D1, pre-dose) of 64% (range 6%-78%, n=18; patients with C2 levels below LLOQ at baseline were excluded from this analysis), suggestive of CDC activity of IgG1-C-E430G in the clinical setting. In most subjects, C2 levels returned to baseline before the following dose, indicating that treatment does not exhaust complement. After administration of the following dose, C2 levels transiently dropped again, and returned to baseline before the subsequent dosing.

In conclusion, decreases in C2 were observed at all dose levels and were transient in most subjects. This confirms the biological activity of IgG1-C-E430G in vivo, and is indicative of CDC activity of IgG1-C-E430G in patients.

TABLE 21
Complement component C2 levels in plasma of patients dosed with IgG1-C-E430G

| | Dose Level | | | | | | |
|---|---|---|---|---|---|---|---|
| | 0.2/0.6 mg/kg | 2 mg/kg | 4 mg/kg | 8 mg/kg | 16 mg/kg | 24 mg/kg | Total |
| C2 (mg/dL) | (N = 1) | (N = 1) | (N = 3) | (N = 3) | (N = 11) | (N = 5) | (N = 24) |
| Baseline | | | | | | | |
| N | 1 | 1 | 3 | 2 | 9 | 4 | 20 |
| Mean | 2.000 | 3.100 | 1.917 | 2.350 | 1.933 | 1.875 | 2.023 |
| Std Dev | N/E | N/E | 1.1857 | 0.9192 | 0.2828 | 0.3862 | 0.5764 |
| Median | 2.000 | 3.100 | 2.100 | 2.350 | 1.800 | 1.800 | 1.900 |
| Min, Max | 2.00, 2.00 | 3.10, 3.10 | 0.65, 3.00 | 1.70, 3.00 | 1.60, 2.50 | 1.50, 2.40 | 0.65, 3.10 |
| Maximum | | | | | | | |
| change | | | | | | | |
| N | 1 | 1 | 3 | 2 | 8 | 4 | 19 |
| Mean | −1.350 | −1.800 | −0.883 | −1.700 | −0.813 | −1.225 | −1.084 |
| Std Dev | N/E | N/E | 0.7751 | 0.9192 | 0.9960 | 0.3862 | 0.8017 |
| Median | −1.350 | −1.800 | −1.200 | −1.700 | −1.150 | −1.150 | −1.200 |
| Min, Max | −1.35, −1.35 | −1.80, −1.80 | −1.45, 0.00 | −2.35, −1.05 | −1.85, 0.90 | −1.75, −0.85 | −2.35, 0.90 |
| Maximum | | | | | | | |
| percentage | | | | | | | |
| change | | | | | | | |
| N | 1 | 1 | 3 | 2 | 8 | 4 | 19 |
| Mean | −67.5000 | −58.0645 | −36.3492 | −70.0490 | −38.7578 | −64.2844 | −49.5742 |
| Std Dev | N/E | N/E | 34.66828 | 11.71579 | 52.11096 | 6.85954 | 37.45303 |
| Median | −67.5000 | −58.0645 | 40.0000 | −70.0490 | −63.8889 | −63.7771 | −63.8889 |
| Min, Max | −67.500, 67.500 | −58.065, −58.065 | −69.048, 0.000 | −78.333, 61.765 | −74.000, 52.941 | −72.917, −56.667 | −78.333, 52.941 |

Cutoff date: 3 Oct. 2022

### Example 7—Evaluation of Complement Lytic Activity in Serum of Patients Dosed with IgG1-C-E430G

### Methods

Blood sampling for analysis of complement lytic activity (CH50) in serum was performed according to the scheme shown in Table 20.

The complement lytic activity (CH50) in serum of patients dosed with IgG1-C-E430G was evaluated by a spectrophotometric assay using the Autokit CH50 (FUJIFILM Wako, Richmond, VA; cat. no. 995-40801). Therefore, 10 μL of serum obtained from patients dosed with IgG1-C-E430G was mixed with 250 μL liposome (Reagent 1) and incubated for 5 min at 37° C. Then 125 μL of substrate (Reagent 2) was added and incubated for another 5 min at 37° C. Finally, the absorbance at 340 nm was measured on a Beckman Coulter AU680 system (Brea, CA). The absorbance increase is proportional to the complement activity in the serum sample.

Results: Complement Lytic Activity in Serum of Patients Dosed with IgG1-C-E430G

Table 22 show a transient decrease in total complement lytic activity (CH50) after administration of the first dose of IgG1-C-E430G at all evaluable doses (8-24 mg/kg), and transient in most subjects. The median peak reduction from baseline (C1 D1, pre-dose) was 53% (range 2%-92%, n=11). This suggests CDC activity of IgG1-C-E430G in the clinical setting. Complement parameters rapidly returned to baseline level in most subjects and remained at baseline levels after subsequent dosing, indicating that treatment does not exhaust complement.

In conclusion, transient decreases in complement lytic activity (CH50) were observed in the peripheral blood of patients dosed with IgG1-C-E430G at all dose levels evaluated. This confirms the biological activity of IgG1-C-E430G in vivo, and is indicative of CDC activity of IgG1-C-E430G in patients.

TABLE 22
Complement lytic activity in serum of patients dosed with IgG1-C-E430G

| | Dose Level | | | | | | |
|---|---|---|---|---|---|---|---|
| | 0.2/0.6 mg/kg | 2 mg/kg | 4 mg/kg | 8 mg/kg | 16 mg/kg | 24 mg/kg | Total |
| CH50 (U/mL) | (N = 1) | (N = 1) | (N = 3) | (N = 3) | (N = 11) | (N = 5) | (N = 24) |
| Baseline | | | | | | | |
| N | 0 | 0 | 10 | 2 | 8 | 5 | 15 |
| Mean | | | | 60.0 | 53.0 | 60.0 | 56.3 |
| Std Dev | | | | 0.00 | 13.64 | 0.00 | 10.30 |
| Median | | | | 60.0 | 60.0 | 60.0 | 60.0 |
| Min, Max | | | | 60, 60 | 23, 60 | 60, 60 | 23, 60 |
| Maximum | | | | | | | |
| change | | | | | | | |
| N | 0 | 0 | 10 | 2 | 8 | 5 | 15 |
| Mean | | | | −25.5 | −15.1 | −20.4 | −18.3 |
| Std Dev | | | | 12.02 | 27.30 | 20.79 | 22.83 |
| Median | | | | −25.5 | −21.5 | −19.0 | −19.0 |
| Min, Max | | | | −34, −17 | −55, 24 | −43, 0 | −55, 24 |
| Maximum | | | | | | | |
| percentage | | | | | | | |
| change | | | | | | | |
| N | 0 | 0 | 10 | 2 | 8 | 5 | 15 |
| Mean | | | | −42.5000 | −15.6742 | −34.0000 | −25.3596 |
| Std Dev | | | | 20.03469 | 63.17567 | 34.65304 | 49.89575 |
| Median | | | | −42.5000 | −35.8333 | −31.6667 | −31.6667 |
| Min, Max | | | | −56.667, −28.333 | −91.667, 104.348 | −71.667, 0.000 | −91.667, 104.348 |

Cutoff date: 3 Oct. 2022

### Example 8—Evaluation of Cytokine Levels in Plasma of Patients Dosed with IgG1-C-E430G

### Methods

Cytokine levels in plasma of patients dosed with IgG1-C-E430G was evaluated by a custom made sandwich immunoassay using 10-spot MULTI-SPOT® plates pre-coated with capture antibodies for IL-2, IL-6, IL-8, IL-10, TNF-α, and IFN-γ (V-PLEX Custom Human Biomarkers; Meso Scale Diagnostics, Rockville, AR; Cat #K151A9H-2). First, plates were washed 3 times with 150 μL/well Wash Buffer (Meso Scale Diagnostics; cat. no. R61AA-1). Then 50 μL/well of plasma sample was added per well, which was incubate at room temperature for 2 h while shaking. After incubation, wells were washed 3 times with Wash Buffer and 25 μL/well of SULFO-TAG-conjugated, detection antibodies (anti-IL-2, cat. no. D21QQ; anti-IL-6, cat. no. D21AK; anti-IL-8, cat. no. D21AN; anti-IL-10, cat. no. D21QU; anti-TNF-α, cat. no. D21BH; anti-IFN-γ, cat. no. D21QO; all from Meso Scale Diagnostics) were added and incubated at room temperature for 2 h while shaking. Finally, wells were washed 3 times with Wash Buffer and 150 μL/Uwell of 2× Read Buffer T (Meso Scale Diagnostics; cat. no. R92TC-3) was added. Plates were read on a MESO® QuickPlex SQ 120 SN instrument (Meso Scale Diagnostics; Ser. No. 1300170726922). The instrument measures the intensity of emitted light to provide a quantitative measure of cytokines in the plasma sample.

Results: Cytokine Levels in Plasma of Patients Dosed with IgG1-C-E430G

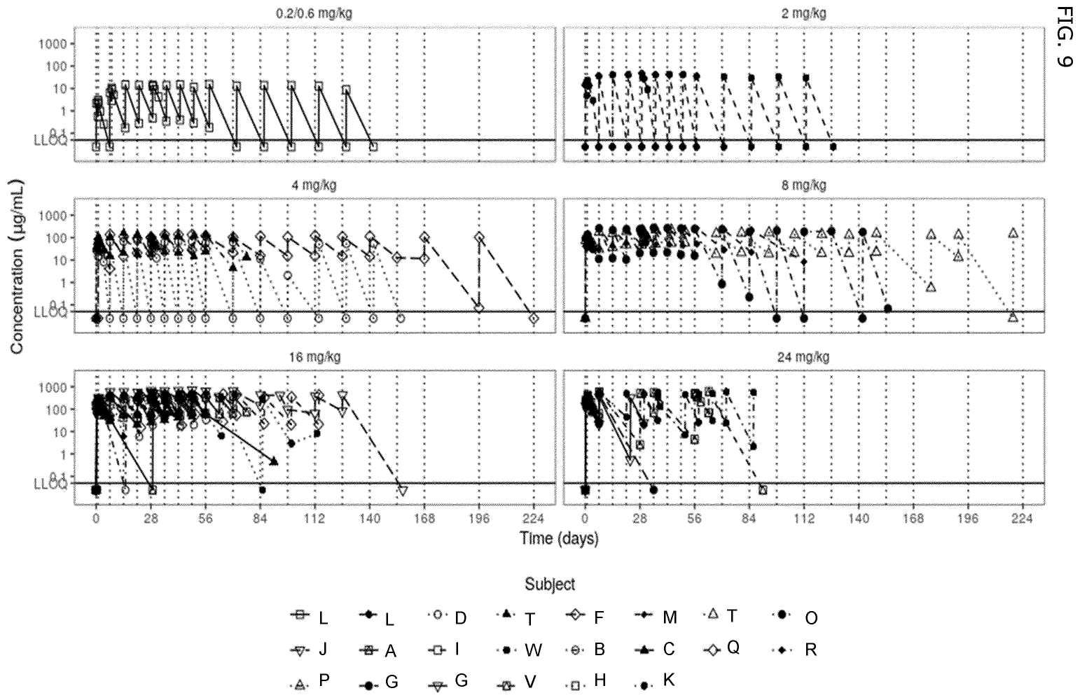

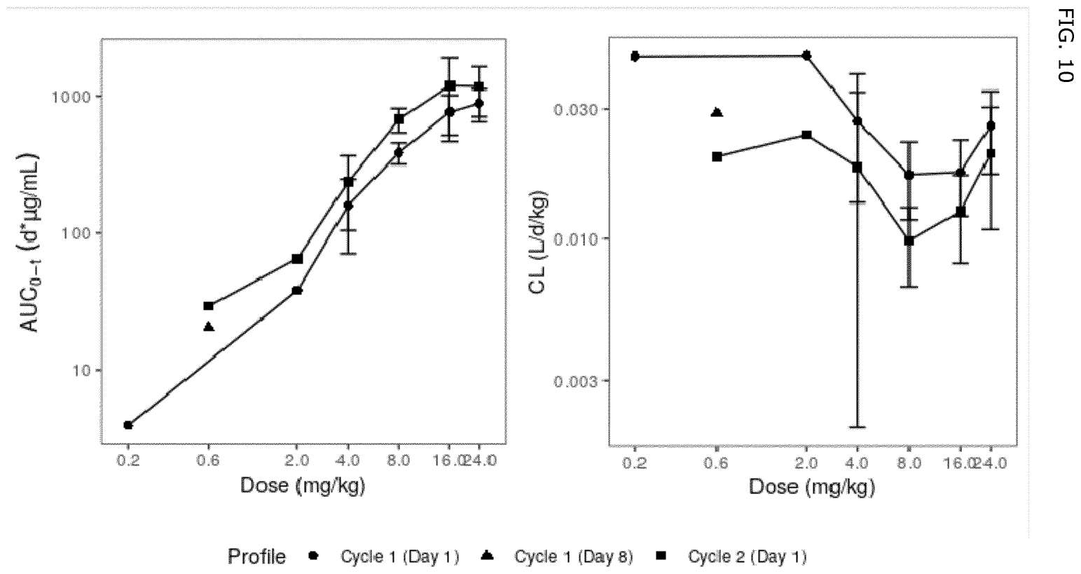

FIG. 7 shows that cytokine levels of IL-2, IL-6, IL-8, IL-10, IFNγ, and TNFα in plasma of patients dosed with IgG1-C-E430G generally remained low across all dose levels, with higher variation between individual subjects at higher (≥16 mg/kg) dose levels.

### Example 9—Evaluation of the Pharmacokinetics of IgG1-C-E430G in RRMM patients

Methods: Determining Pharmacokinetic Parameters in Patients Dosed with IgG1-C-E430G

The pharmacokinetics were evaluated for patients dosed with 0.2/0.6 to 24 mg/kg IgG1-C-E430G in the dose escalation part of the first-in-human trial of IgG1-C-E430G (NCT04824794).

As of the data cutoff date of 29 Jun. 2022, pharmacokinetic data were available from 22 subjects with RRMM dosed with IgG1-C-E430G in the Dose Escalation part (0.2/0.6 to 24 mg/kg) of the first-in-human trial.

Blood samples for determination of serum concentrations were drawn from patients according to the scheme shown in Table 23. Blood was collected into 4 ml serum separation tubes. After incubation at room temperature (30 min) and centrifugation step (10 min at 1500 g), serum is transferred to cryo tubes and stored at <−65° C.

TABLE 23
Schedule for blood sampling. Days in the 28-day treatment cycle.
Timepoints relative to infusion, sampling window in brackets.

| Day | Timepoint |
|---|---|
| Day 1 | Predose (−30 min) |
| | End of infusion (+5 min) |
| | End of infusion +2 h (±15 min) ¹ |
| | End of infusion +4 h (±30 min) ¹ |
| Day 2 | End of infusion +24 h (±2 h) ¹ |
| | Predose (−30 min) Day 22 |
| | End of Day 2 infusion (+5 min) ² |
| | End of Day 2 infusion +2 h (±15 min) ² |
| | End of Day 2 infusion +4 h (±30 min) ² |
| Day 3 | End of Day 2 infusion +24 h (±2 h) ² |
| Day 4 | End of infusion +72 h (±24 h) ¹ |
| Day 5 | End of Day 2 infusion +72 h (±24 h) ² |
| Day 8 | Predose (−30 min) ³ |
| | End of infusion (+5 min) ³ |
| Day 9 | Predose (−30 min) ^{2, 5} |
| | End of infusion (+5 min) ^{2, 5} |
| | End of infusion +4 h (±30 min) ^{2, 5} |
| Day 10 | End of Day 9 infusion +24 h (±2 h) ^{2, 5} |
| Day 15 | Predose (−30 min) ⁴ |
| | End of infusion (+5 min) ⁴ |
| Day 22 | Predose (−30 min) ³ |
| | End of infusion (+5 min) ³ |

¹ Cycle 2 only.
² Cycle 1 only.
³ Cycle 1 and 2 only.
⁴ Cycle 1 to 6 only.
⁵ For 0.2/0.6 mg/kg patient only.

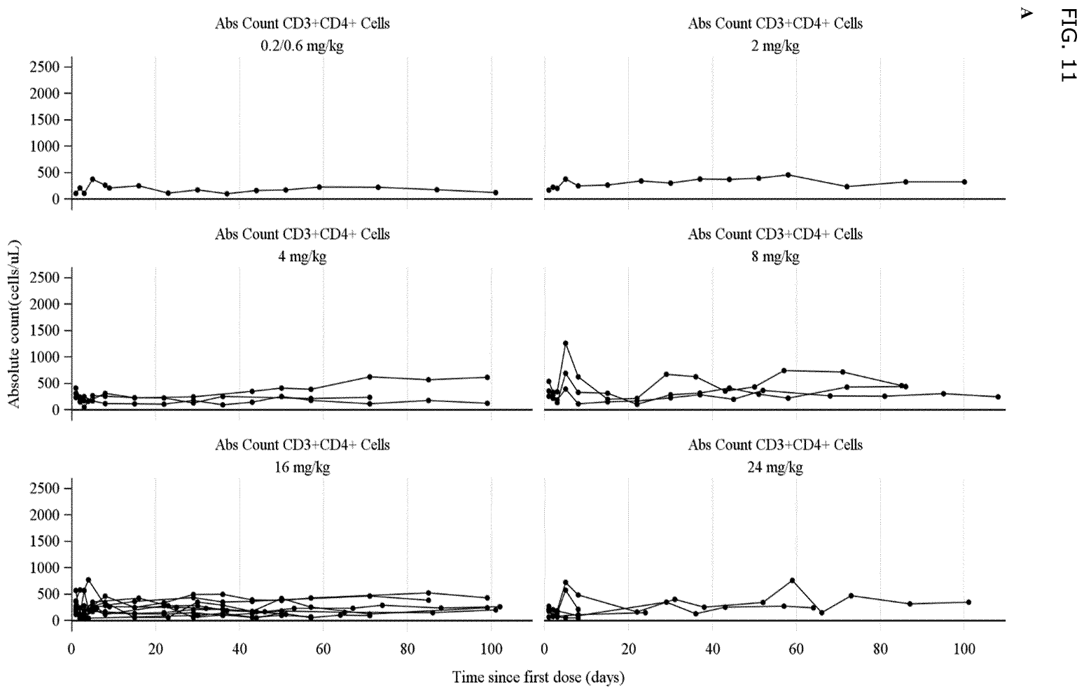

Serum IgG1-C-E430G concentrations were determined using an electrochemiluminescence sandwich immunoassay (ECLIA) method. The assay principle is depicted FIG. 8. The validated analytical assay range is from 0.05 μg/ml to 3.20 μg/ml with a minimal required dilution (MRD) of 40. IgG1-C-E430G can be quantified up to 990 μg/ml in neat serum when additional validated dilution factors are applied.

To capture and detect IgG1-C-E430G in human serum, two distinct anti-idiotype antibodies were used. The capture anti-idiotype antibody was coated onto an ECLIA multi-array plate (75 μl/well, diluted to 1 μg/ml in Phosphate-buffered saline (PBS, Sigma)) by overnight incubation at 4° C. Subsequently, plates were washed three times with PBS containing 0.01%, w/v, Tween-20 (Sigma), followed by incubation with 150 μl/well Scytek Laboratories Super Block buffer (assay buffer), 1 h at room temperature. 75 μl of samples containing the IgG1-C-E430G (calibrators, quality control and study samples) diluted to MRD in assay buffer, were added to the plates, and incubated for 1 h at room temperature. Bound IgG1-C-E430G was detected by incubation with SULFO-TAG labeled detection anti-idiotype antibody (75 μl/well diluted to 1 μg/ml in assay buffer) for 1 h at room temperature. After incubation, plates were washed three times and Read Buffer T (MSD) was added. The light emitted by the SULFO-TAG upon electrochemical stimulation at the electrode surfaces of the multi-array plate was measured in a MDS multi-array plate reader at 620 nm. The amount of light emitted is an indirect readout for the concentration of IgG1-C-E430G in the sample.

PK parameters were calculated using the Phoenix 64 software package (version 8.2, Certara USA, Inc., Princeton, NJ) by noncompartmental methods consistent with the route of administration (intravenous infusion). The following parameters were derived from the serum concentration-time profiles of the administrations on Cycle 1 Day 1, Cycle 1 Day 8 (only patient E, dosed 0.2-0.6 mg/kg) and Cycle 2 Day 1:
AUC₀₋ₜ—area under the serum concentration-time curve between the start of administration and the last time point before the next administration with a quantifiable concentration (d*ug/mL), calculated using the linear up, log-linear down trapezoidal method.
CL—clearance (L/d/kg), calculated if feasible as Dose/(AUC₀₋ₜ+Cₜ/λ_{z}), where λ_{z} (d⁻¹) is the slope of the log-concentration-time curve determined by regression using a minimum of three observations in the elimination phase and Cₜ is the predicted rather than the observed concentration at the last time point with a quantifiable concentration.

PK profiles included all PK observations from immediately prior to the administration to the start of the next administration, where a split dose between two consecutive days is considered one administration. A value of 0 was imputed for all pretreatment concentrations and for predose concentrations below lower limit of quantification (BLQ) at the start of subsequent PK profiles. All other observations BLQ were omitted. PK parameters were summarized as means and standard deviations by dose and PK profile.

Results: PK Observations in Patients Dosed with IgG1-C-E430G

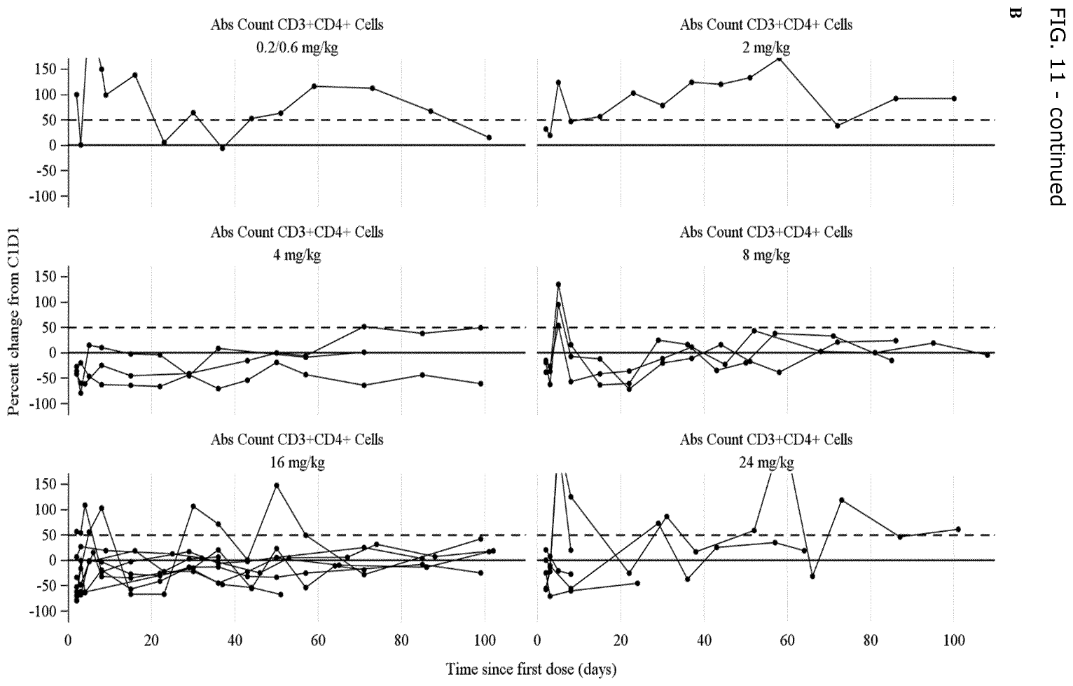

FIG. 9 shows that the peak concentrations at end of infusion increase with increasing dose and are followed by a two-phase decline. PK profiles at 16 mg/kg were more consistent between subjects and exposure was better maintained during biweekly dosing compared to lower dose levels. Peak concentrations show limited accumulation in upon weekly dosing from Day 8 onward at all dose levels, suggesting a faster total clearance across dose levels than typical for IgG1 antibody therapeutics. An increase in predose concentrations over the course of weekly dosing was observed in some patients, most notable in patients with a minimal response or better (e.g. patient F, T, J, C), indicative of a decreasing impact of target-mediated drug disposition over time potentially due to target cell depletion.

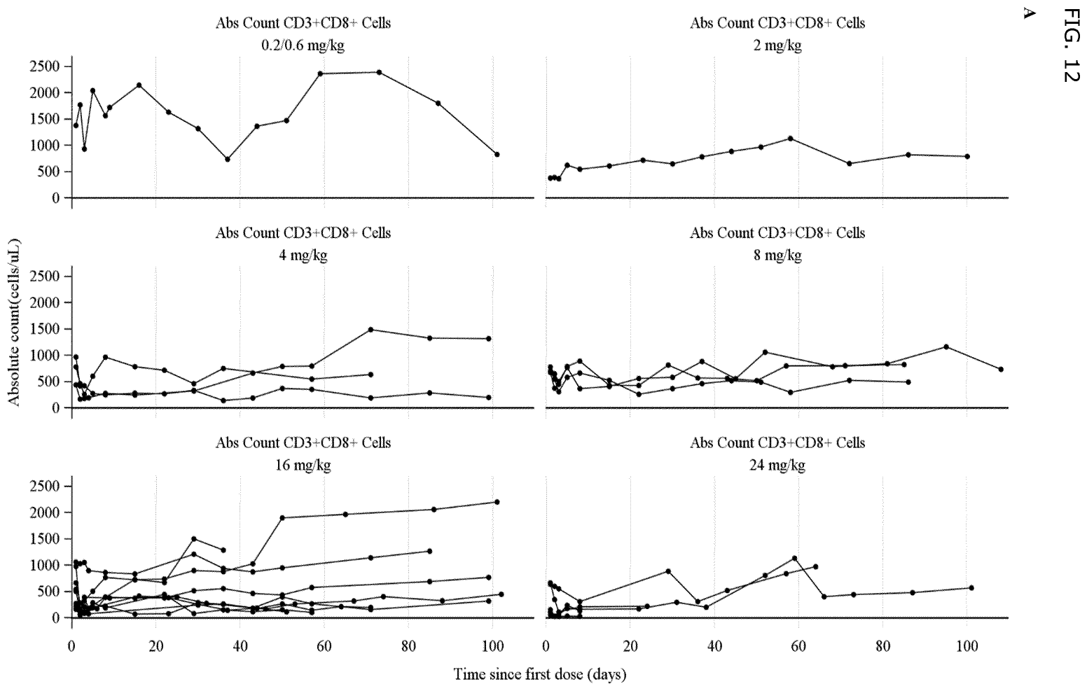

AUC₀₋ₜ and CL calculated for patients dosed with IgG1-C-E430G after the first and fifth administration, and after the second administration for the patient dosed 0.2/0.6 mg/kg, are plotted against dose in FIG. 10 and summarized by dose level in Table 24. These data show that the AUC₀₋ₜ after the first and fifth administration increased in a more than dose-proportional manner up to 4 mg/kg and roughly proportional to dose from 4 mg/kg upward. A substantial overlap was observed in AUC₀₋ₜ between subjects dosed at 16 mg/kg and 24 mg/kg. Initial CL was faster at doses below mg/kg and was roughly constant from 4 mg/kg upward. These results suggest an impact of target-mediated drug disposition during weekly dosing at dose levels below 4 mg/kg and a high degree of target saturation at or above 4 mg/kg. The CL observed for NgG)-C-E430G at dose levels between 4 and 24 mg/kg are higher than observed for prior anti-CD38 antibodies at similar dose levels.

In summary, the PK of IgG1-C-E430G was characterized by a more than proportional increase of AUC₀₋ₜ with dose between 0.2 and 4 mg/kg consistent with target-mediated drug disposition and a roughly proportional increase at higher dose levels suggesting a high degree of target saturation during weekly dosing at doses ≥4 mg/kg. CL was faster than typical for an IgG1 antibody and faster than prior anti-CD38 antibodies at similar dose levels. Some patients showed an increase in predose concentrations over the course of weekly dosing, suggestive of target depletion over time.

TABLE 24
PK parameters derived for patients dosed with IgG1-C-E430G

| Dose Level | | | | | |
|---|---|---|---|---|---|
| 0.2/0.6 mg/kg | 2 mg/kg | 4 mg/kg | 8 mg/kg | 16 mg/kg | 24 mg/kg |
| (N = 1) | (N = 1) | (N = 3) | (N = 3) | (N = 11) | (N = 5) |

Cycle 1 (Day 1)

| | | | | | | |
|---|---|---|---|---|---|---|
| AUC₀₋ₜ (d*μg/mL) | | | | | | |
| n | 1 | 1 | 3 | 3 | 9 | 5 |
| Mean | 3.93 | 38.2 | 159 | 386 | 764 | 884 |
| | 10 | | | | | |
| Std Dev | NA | NA | 88.2 | 68.6 | 246 | 237 |
| CL (L/d/kg) | | | | | | |
| n | 1 | 1 | 3 | 3 | 7 | 5 |
| Mean | 0.0469 | 0.0473 | 0.0270 | 0.0172 | 0.0176 | 0.0261 |
| Std Dev | NA | NA | 0.0134 | 0.00548 | 0.00551 | 0.00878 |

Cycle 1 (Day 8)

| | | | | | | |
|---|---|---|---|---|---|---|
| AUC₀₋ₜ (d*μg/mL) | | | | | | |
| n | 1 | 0 | 0 | 0 | 0 | 0 |
| Mean | 20.5 | | | | | |
| Std Dev | NA | | | | | |
| CL (L/d/kg) | | | | | | |
| n | 1 | 0 | 0 | 0 | 0 | 0 |
| Mean | 0.0289 | | | | | |
| Std Dev | NA | | | | | |

Cycle 2 (Day 1)

| | | | | | | |
|---|---|---|---|---|---|---|
| AUC₀₋ₜ (d*μg/mL) | | | | | | |
| n | 1 | 1 | 3 | 3 | 9 | 2 |
| Mean | 29.3 | 65.2 | 235 | 675 | 1189 | 1176 |
| Std Dev | NA | NA | 131 | 135 | 721 | 472 |
| CL (L/d/kg) | | | | | | |
| n | 1 | 1 | 3 | 3 | 9 | 2 |
| Mean | 0.0201 | 0.0242 | 0.0183 | 0.00985 | 0.0126 | 0.0206 |
| Std Dev | NA | NA | 0.0163 | 0.00319 | 0.00451 | 0.00981 |

### Example 10—Further Clinical Efficacy Evaluation from the Dose Escalation

### Methods: Dose Escalation—Efficacy

The trial design for IgG-C-E430G is described in Example 2 and a schematic is shown in FIG. 2. In the dose escalation part of the trial, subjects with RRMM were treated with IgG1-C-E430G across 6 dose levels in this first-in-human trial. The reporting period is from October 2022 through the data cut-off date of 14 Aug. 2023.

### Results

During the current reporting period, additional follow-up data were collected on the subjects who remained in the dose escalation part of the IgG-C-E430G trial. Among the 24 subjects in this part of the trial, 2 subjects (8.3%) continued to receive trial treatment as of the data cut-off date. Thirteen subjects (54.2%) discontinued treatment and remained on trial. Nine subjects (37.5%) discontinued treatment and withdrew from the trial or died. The median duration of IgG1-C-E430G full-dose infusion was 3.8 hours (range: 1.1-4.6).

Overall, the efficacy results for the dose escalation part as of 14 Aug. 2023 were unchanged from those in the previous reporting period, ie, before October 2022. See Example 3 for the best overall responses across the 6 dose levels administered during that reporting period.

In summary, the efficacy data in the current period confirm that IgG1-C-E430G has clinical activity in patients with anti-CD38 mAb-naïve as well as anti-CD38 mAb pretreated RRMM.

### Example 11—Further Clinical Safety Evaluation from the Dose Escalation

### Methods: Dose Escalation—Safety

The trial design for IgG-C-E430G is described in Example 2 and a schematic is shown in FIG. 2. In the dose escalation part of the trial, subjects with RRMM were treated with IgG1-C-E430G across 6 dose levels in this first-in-human trial. The reporting period is from October 2022 through the data cut-off date of 14 Aug. 2023.

### Results

Overall, the safety results for the dose escalation part of the IgG-C-E430G trial, as of the data cut-off date, were unchanged from those in the previous reporting period, ie, before October 2022. See Example 4 for summaries of TEAEs, related TEAEs, serious TEAEs, related serious TEAEs, and AESIs across the 6 dose levels administered during the previous reporting period.

In summary, the safety data collected from October 2022 through 14 Aug. 2023 confirm that IgG1-C-E430G has an acceptable safety profile, with no tumor lysis syndrome or cytokine release syndrome events.

### Example 12—Clinical Efficacy Evaluation in Expansion Part A

### Methods: Efficacy—Expansion Part A

The trial design for IgG1-C-E430G, including expansion part A and preliminary efficacy, are described in Example 2. FIG. 2 shows a schematic overview of the IgG-C-E430G trial design.

In this part of the trial, subjects with RRMM who were anti-CD38 mAb-naive were treated with IgG1-C-E430G at the RP21D identified for RRMM from the dose escalation part of the trial. As of the data cutoff date, 14 Aug. 2023, efficacy data were available from these subjects who were dosed with 16 mg/kg IgG-C-E430G in the expansion part A.

### Results

Table 25 shows the best overall response for 11 subjects with RRMM who were anti-CD38 mAb-naïve and treated with 16 mg/kg of anti-CD38 mAb in this trial. Of the 11 subjects, 8 (72.7%) received 2 cycles of anti-CD38 mAb. The best overall responses among the 11 subjects were complete response in subject (9.1%), very good partial response in 2 subjects (18.2)%, partial response in 3 subjects (27.3%), minimal response in 2 subjects (18.2%), stable disease in 1 subject (9.1%), and 2 subjects (18.2%) were not evaluable.

TABLE 25
Best Overall Response - Expansion
Part A - RRMM Anti-CD38 mAb-naive

| | 16 mg/kg | |
|---|---|---|
| Best Overall Response, n(%) | (N = 11) | |
| Stringent complete response (sCR) | 0 | |
| Complete response (CR) | 1 | (9.1%) |
| Very good partial response (VGPR) | 2 | (18.2%) |
| Partial response (PR) | 3 | (27.3%) |
| Minimal response (MR) | 2 | (18.2%) |
| Stable disease (SD) | 1 | (9.1%) |
| Progressive disease (PD) | 0 | |
| Not Evaluable (NE) | 2 | (18.2%) |
| CR or better | 1 | (9.1%) |
| (95% CI) [a] | (0.2%, 41.3%) | |
| Overall Response Rate (ORR) [b] | 6 | (54.5%) |
| (95% CI) [a] | (23.4%, 83.3%) | |
| VGPR or better | 3 | (27.3%) |
| (95% CI) [a] | (6.0%, 61.0%) | |
| Clinical benefit [b] | 8 | (72.7%) |

| | |
|---|---|
| (95% CI) [a] | (39.0%, 94.0%) |

[a] Based on the Clopper and Pearson method.
[b] ORR includes subjects with best response of CR and PR; Clinical benefit includes ORR and MR

In summary, the efficacy data collected from October 2022 through 14 Aug. 2023 of the expansion part A of the IgG-C-E430G trial showed that IgG1-C-E430G has clinical activity in anti-CD38 mAb-naive RRMM patients.

### Example 13—Clinical Safety Evaluation in Expansion Part A

### Methods: Expansion Part A—Clinical Safety

The trial design for IgG1-C-E430G, including expansion part A, and the assessments for safety are described in Example 2. FIG. 2 shows a schematic overview of the IgG-C-E430G trial design.

In this part of the trial, subjects with RRMM who were anti-CD38 mAb-naive were treated with IgG1-C-E430G at the RP2D identified for RRMM from the dose escalation part of the trial. As of the data cutoff date, 14 Aug. 2023, safety data were available from these subjects who were dosed with 16 mg/kg IgG1-C-E430G in the expansion part A.

### Results

As of 14 Aug. 2023, 9 of 11 subjects (81.8%) in the Expansion Part A part experienced at least 1 treatment-emergent adverse event (TEAE) (Table 26). The most common TEAEs (in ≥20% of subjects) reported in this part of the trial were neutropenia (6 subjects; 54.5%); and anaemia, headache, IRR, thrombocytopenia, and upper respiratory tract infection (3 subjects per each event; 27.3%). Eight subjects (72.7%) experienced a TEAE considered as related to IgG1-C-E430G (Table 27). The related TEAEs reported most often were neutropenia (6 subjects; 54.5%), infusion related reaction (3 subjects; 27.3%), anaemia (2 subjects; 18.2%), and thrombocytopenia (2 subjects; 18.2%).

Serious TEAEs were reported in 5 (45.5%) subjects (Table 28). The most common serious TEAEs were cardiac arrest (2 subjects; 18.2%); and anaemia, brain injury, death, respiratory tract infection, seizure, and upper respiratory tract infection (1 subject per event; 9.1%). Two subjects (18.2%) experienced a serious TEAE considered related to IgG1-C-E430G: anaemia, brain injury, cardiac arrest, and death (1 subject per event; 9.1%) (Table 29).

Three subjects (27.3%) experienced IRR at the 16 mg/kg dose level (Table 30).

No cytokine release syndrome events were reported.

Three subjects (27.3%) discontinued trial treatment and remained in the trial, and 5 subjects (45.5%) discontinued trial treatment and withdrew or died. Reasons for treatment discontinuation include disease progression (3 subjects; 27.3%), clinical progression (1 subject; 9.1%), and AE (4 subjects; 36.4%). The primary reasons for trial discontinuation include death (4 subjects; 36.4%) and lost to follow up (1 subject; 9.1%).

TABLE 26
Treatment Emergent Adverse Events -Expansion
Part A - RRMM anti-CD38 mAb-naive

| | 16 mg/kg |
|---|---|
| | (N = 11) |
| Number of subjects with at least one TEAE, n(%) | 9 (81.8%) |
| Neutropenia | 6 (54.5%) |
| Anaemia | 3 (27.3%) |
| Headache | 3 (27.3%) |
| Infusion related reaction | 3 (27.3%) |
| Thrombocytopenia | 3 (27.3%) |
| Upper respiratory tract infection | 3 (27.3%) |
| Cardiac arrest | 2 (18.2%) |
| Cough | 2 (18.2%) |
| Hypokalemia | 2 (18.2%) |
| Nausea | 2 (18.2%) |
| Pyrexia | 2 (18.2%) |
| Respiratory tract infection | 2 (18.2%) |
| Acidosis | 1 (9.1%) |
| Alanine aminotransferase increased | 1 (9.1%) |
| Aspartate aminotransferase increased | 1 (9.1%) |
| Atrial fibrillation | 1 (9.1%) |
| Blood creatinine increased | 1 (9.1%) |
| Bradycardia | 1 (9.1%) |
| Brain injury | 1 (9.1%) |
| COVID-19 | 1 (9.1%) |
| Candida infection | 1 (9.1%) |
| Chest discomfort | 1 (9.1%) |
| Constipation | 1 (9.1%) |
| Death | 1 (9.1%) |
| Dermatitis allergic | 1 (9.1%) |
| Diabetes mellitus | 1 (9.1%) |
| Fungal infection | 1 (9.1%) |
| Hyperglycaemia | 1 (9.1%) |
| Hyperkalaemia | 1 (9.1%) |
| Hypernatraemia | 1 (9.1%) |
| Hypertransaminasaemia | 1 (9.1%) |
| Hyperuricaemia | 1 (9.1%) |
| Hypoalbuminaemia | 1 (9.1%) |
| Hypocalcaemia | 1 (9.1%) |
| Hypomagnesaemia | 1 (9.1%) |
| Hyponatraemia | 1 (9.1%) |
| Hypophosphataemia | 1 (9.1%) |
| Hypotension | 1 (9.1%) |
| International normalised ratio increased | 1 (9.1%) |
| Lymphocyte count decreased | 1 (9.1%) |
| Muscle spasms | 1 (9.1%) |
| Musculoskeletal chest pain | 1 (9.1%) |
| Myalgia | 1 (9.1%) |
| Obesity | 1 (9.1%) |
| Pharyngitis | 1 (9.1%) |
| Platelet count decreased | 1 (9.1%) |
| Pneumonia | 1 (9.1%) |
| Rash | 1 (9.1%) |
| Rash pruritic | 1 (9.1%) |
| Respiratory failure | 1 (9.1%) |
| Rhinitis | 1 (9.1%) |
| Seizure | 1 (9.1%) |
| Sinusitis | 1 (9.1%) |
| Skin papilloma | 1 (9.1%) |
| Steroid diabetes | 1 (9.1%) |
| Tachycardia | 1 (9.1%) |
| Troponin I increased | 1 (9.1%) |
| Urinary tract infection | 1 (9.1%) |
| Weight decreased | 1 (9.1%) |
| Wheezing | 1 (9.1%) |
| White blood cell count decreased | 1 (9.1%) |

Percentages are based on N, Multiple adverse events with a same preferred term are only counted once for each subject.
Coded with MedDRA v.26.1.
Treatment Emergent Adverse Events are AEs that started or pre-existing AEs that worsened on/after the first IMP and within 30 days of last IMP.

TABLE 27
Related Treatment Emergent Adverse Events -
Expansion Part A - RRMM anti-CD38 mAb-naive

| | 16 mg/kg |
|---|---|
| | (N = 11) |
| Number of subjects with at least one related TEAE, n(%) | 8 (72.7%) |
| Neutropenia | 6 (54.5%) |
| Infusion related reaction | 3 (27.3%) |
| Anaemia | 2 (18.2%) |
| Thrombocytopenia | 2 (18.2%) |
| Aspartate aminotransferase increased | 1 (9.1%) |
| Brain injury | 1 (9.1%) |
| Cardiac arrest | 1 (9.1%) |
| Cough | 1 (9.1%) |
| Death | 1 (9.1%) |
| Headache | 1 (9.1%) |
| Hypertransaminasaemia | 1 (9.1%) |
| Hyperuricaemia | 1 (9.1%) |
| Hypoalbuminaemia | 1 (9.1%) |
| Hypocalcaemia | 1 (9.1%) |
| Hypokalaemia | 1 (9.1%) |
| Hypomagnesaemia | 1 (9.1%) |
| Hyponatraemia | 1 (9.1%) |
| Hypophosphataemia | 1 (9.1%) |
| Lymphocyte count decreased | 1 (9.1%) |
| Muscle spasms | 1 (9.1%) |
| Nausea | 1 (9.1%) |
| Platelet count decreased | 1 (9.1%) |
| Skin papilloma | 1 (9.1%) |
| Urinary tract infection | 1 (9.1%) |
| White blood cell count decreased | 1 (9.1%) |

Percentages are based on N, Multiple adverse events with a same preferred term are only counted once for each subject.
Coded with MedDRA v.26.1.
Treatment Emergent Adverse Events are AEs that started or pre-existing AEs that worsened on/after the first IMP and within 30 days of last IMP.
Related: if recorded as Related to IMP by the investigator or recording of relationship was missed.

TABLE 28
Serious Treatment Emergent Adverse Events -
Expansion Part A - RRMM anti-CD38 mAb-naive

| | 16 mg/kg |
|---|---|
| | (N = 11) |
| Number of subjects with at least one serious TEAE, n(%) | 5 (45.5%) |
| Cardiac arrest | 2 (18.2%) |
| Anaemia | 1 (9.1%) |
| Brain injury | 1 (9.1%) |
| Death | 1 (9.1%) |
| Respiratory tract infection | 1 (9.1%) |
| Seizure | 1 (9.1%) |
| Upper respiratory tract infection | 1 (9.1%) |

Percentages are based on N, Multiple adverse events with a same preferred term are only counted once for each subject.
Coded with MedDRA v.26.1.
Treatment Emergent Adverse Events are AEs that started or pre-existing AEs that worsened on/after the first IMP and within 30 days of last IMP.

TABLE 29
Related Serious Treatment Emergent Adverse Events -
Expansion Part A - RRMM anti-CD38 mAb-naive

| | 16 mg/kg |
|---|---|
| | (N = 11) |
| Number of subjects with at least one related serious TEAE, n(%) | 2 (18.2%) |
| Anaemia | 1 (9.1%) |
| Brain injury | 1 (9.1%) |
| Cardiac arrest | 1 (9.1%) |
| Death | 1 (9.1%) |

Percentages are based on N, Multiple adverse events with a same preferred term are only counted once for each subject.
Coded with MedDRA v.26.1.
Treatment Emergent Adverse Events are AEs that started or pre-existing AEs that worsened on/after the first IMP and within 30 days of last IMP.
Related: if recorded as Related to IMP by the investigator or recording of relationship was missed.

TABLE 30
Summary of Adverse Events of Special Interest: Infusion-Related
Reaction (IRR) - Expansion Part A - RRMM anti-CD38 mAb-naive

| | 16 mg/kg |
|---|---|
| | (N = 11) |
| Number of subjects with at least one AESI (IRR), n(%) | |
| Total | 3 (27.3%) |
| IRR Grade 1 | 0 |
| IRR Grade 2 | 3 (27.3%) |
| IRR Grade 3 | 0 |
| IRR Grade 4 | 0 |
| IRR Grade 5 | 0 |
| Number of subjects with at least one, n(%) | |
| IRR leading to permanent treatment discontinuation | 0 |
| IRR leading to dose interruption | 3 (27.3%) |

Infusion-related reactions are limited to events flagged as AESI and with a preferred term of Infusion related reaction.

In summary, the safety data collected from October 2022 through 14 Aug. 2023 of the expansion part A of the IgG-C-E430G trial showed that IgG1-C-E430G has a manageable safety profile in anti-CD38 mAb-naive RRMM patients.

### Example 14—Evaluation of Lymphocyte Populations in Whole Blood of Patients Dosed with IgG1-C-E430G in Expansion Part A

### Methods

Lymphocyte populations in whole blood from RRMM patients dosed with 16 mg/kg IgG1-C-E430G were evaluated as described in Example 5. As of the data cutoff date of 14 Aug. 2023, preliminary pharmacodynamic data were available from 10 subjects with RRMM dosed with 16 mg/kg IgG1-C-E430G in the Expansion Part A of the first-in-human trial.

Results: NK Cell Numbers in Peripheral Blood of Patients Dosed with 16 mg/Kg IgG1-C-E430G

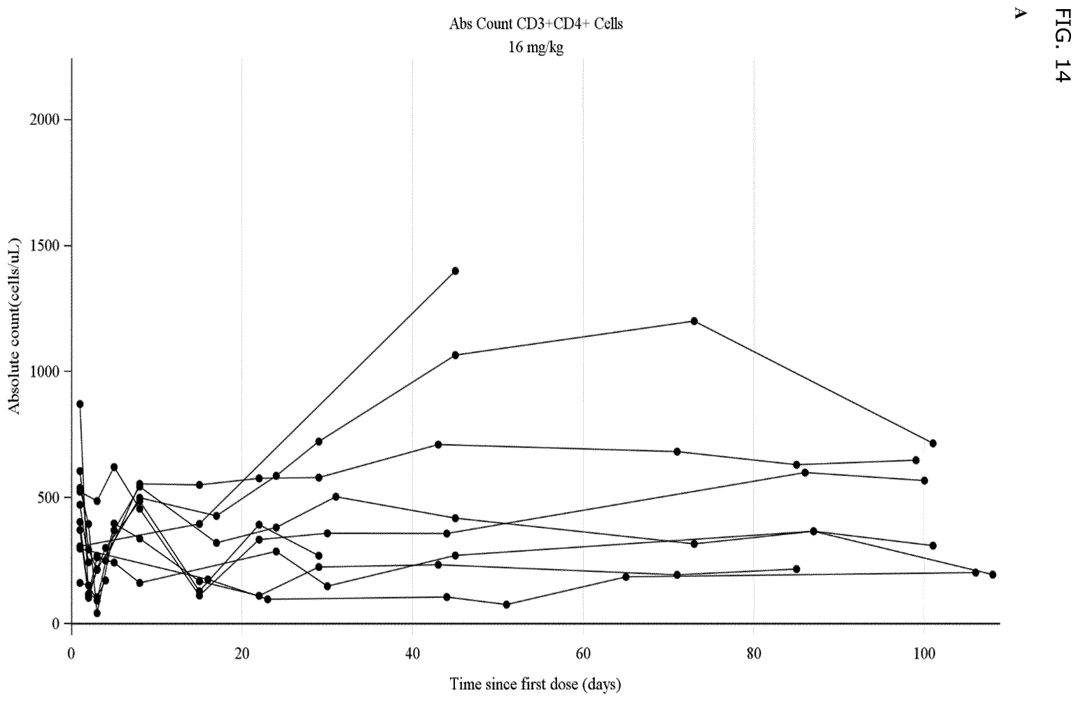

FIG. 13 shows that IgG1-C-E430G administration was associated with a rapid decrease in the number of peripheral blood NK cells (CD3−/CD56+/CD16+ cells). The median maximum percentage reduction in the number of NK cells, compared to baseline (C1D1, pre-dose) was 204% (range −812%-83%, n=11) in evaluable patients. NK cell numbers remained low while on IgG1-C-E430G treatment in majority of patients. Baseline and maximum changes in NK cell numbers and percentages in peripheral blood of patients dosed with IgG1-C-E430G are summarized in Table 31.

In conclusion, prominent NK cell reduction was observed in patients dosed with 16 mg/kg IgG1-C-E430G in expansion part A, similar to observations in the dose escalation phase (Example 5). The observed NK cell decrease confirms biological activity of IgG1-C-E430G in patients and is indicative of the ADCC activity of IgG1-C-E430G.

TABLE 31
NK Cell population in Peripheral Blood in
patients dosed with 16 mg/kg IgG1-C-E430G

| CD3−/CD56+/CD16+ ABS | Dose Level |
|---|---|
| (Cells/uL) | 16 mg/kg |
| Baseline | |
| n | 10 |
| Mean | 319 |
| Std Dev | 300.31 |
| Median | 205 |
| Min, Max | 0.0, 813.0 |
| Maximum change | |
| n | 10 |
| Mean | −306.6 |
| Std Dev | 309.79 |
| Median | −204 |
| Min, Max | −812.0, 83.0 |
| Maximum percentage change | |
| n | 9 |
| Mean | −98 |
| Std Dev | 2.33 |
| Median | −98.9 |
| Min, Max | −100.0, −93.0 |

Cutoff date: 14 Aug. 2023

### Results: T Cell Numbers in Peripheral Blood of Patients Dosed with 16 mg/Kg IgG1-C-E430G

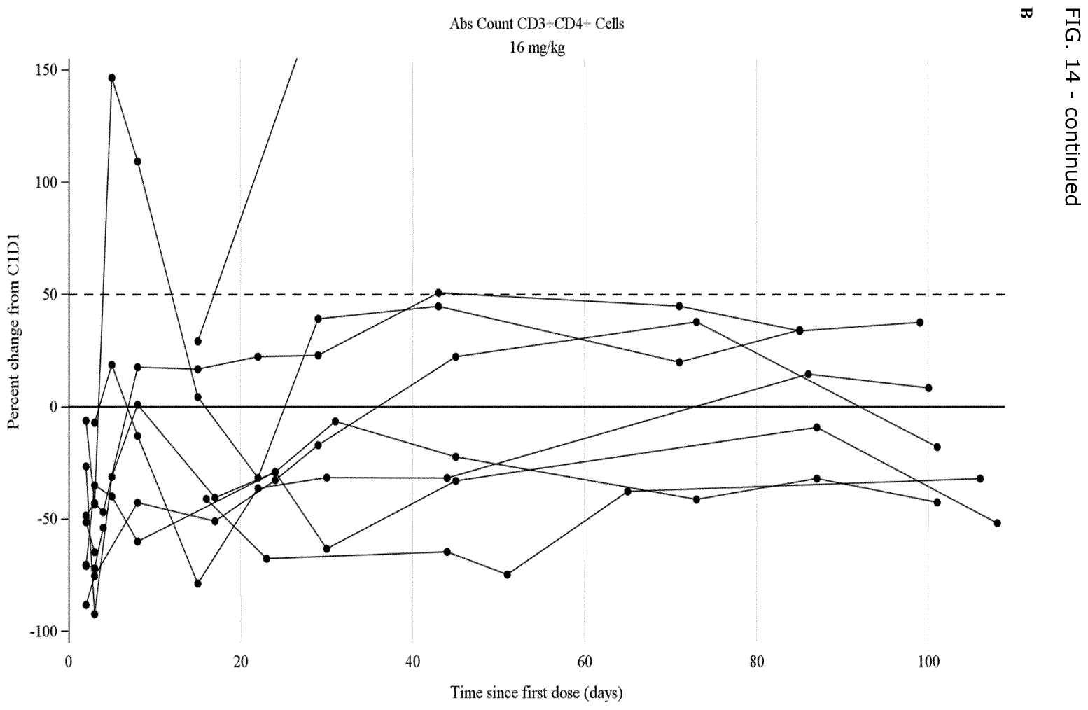

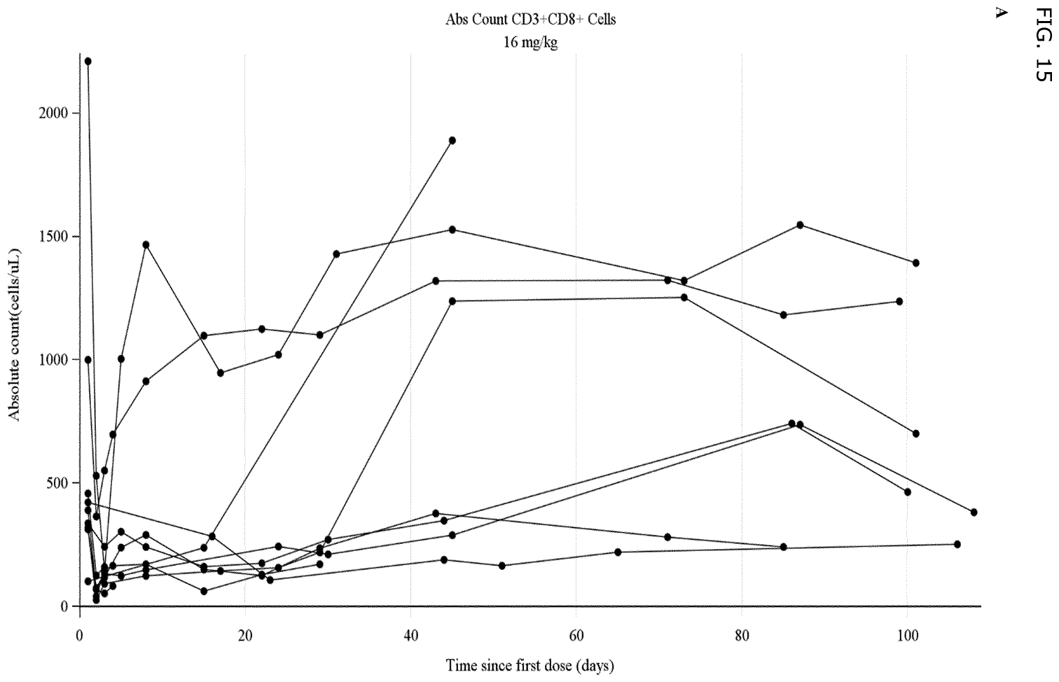

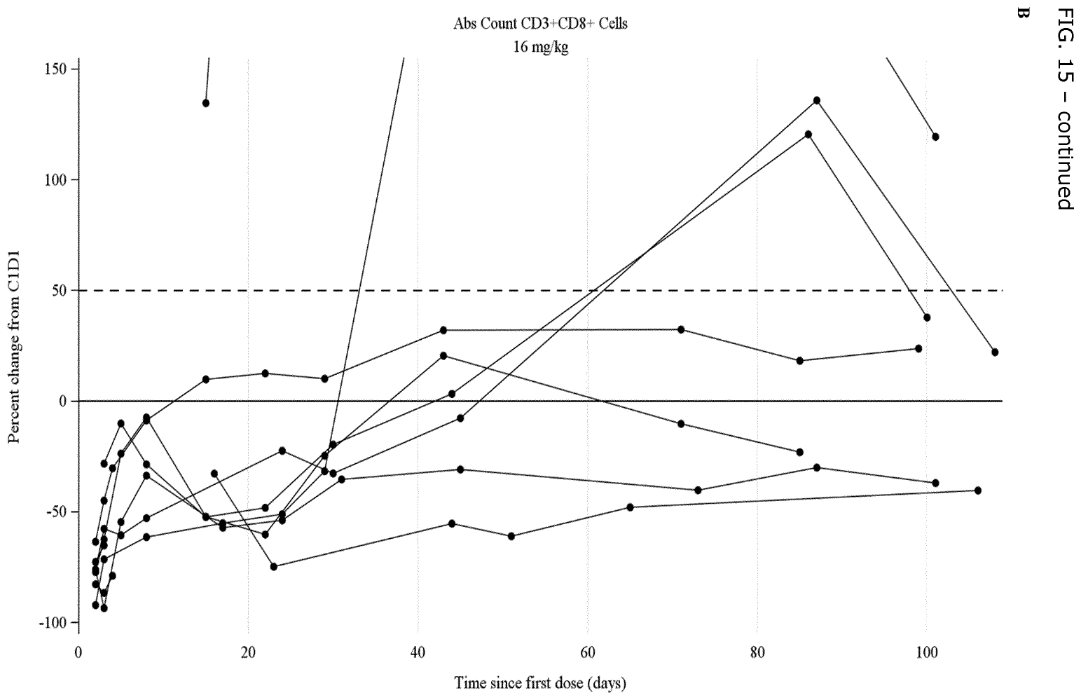

FIG. 14 and FIG. 15 show a transient decrease in CD3+CD4+ and CD3+CD8+ T cells after administration of the first dose of IgG1-C-E430G at 16 mg/kg (data cut off: 14 Aug. 2023). A subsequent increase in the number of peripheral blood CD3+CD4+ T cells (>50% increase from baseline for ≥2 visits) was observed in 1 out 10 evaluable patients (FIG. 14). In addition, an increase in the number of peripheral blood CD3+CD8+ T cells (>50% increase from baseline for ≥2 visits) was observed in 3 out 10 evaluable patients (FIG. 15). Baseline and maximum changes in CD3+CD4+ and CD3+CD8+ T cell numbers and percentages in peripheral blood of patients dosed with IgG1-C-E430G are summarized in Table 32 and Table 33.

In conclusion, after an initial decline, increases in peripheral T cell numbers were observed in a subset of patients dosed with 16 mg/kg IgG1-C-E430G. In particular, CD3+CD8+ T cells showed a significant increase in some of the subjects. The observed T cell expansion confirms biological activity of IgG1-C-E430G in vivo, and is indicative of the immunomodulatory activity of IgG1-C-E430G in patients.

TABLE 32
CD4+ T Cells in Peripheral Blood
of patients dosed with 16 mg/kg IgG1-C-E430G

| CD3+CD4+ ABS | Dose Level |
|---|---|
| (Cells/uL) | 16 mg/kg |
| Baseline | |
| n | 10 |
| Mean | 454.6 |
| Std Dev | 197.4 |
| Median | 437 |
| Min, Max | 161.0, 871.0 |
| Maximum change | |
| n | 10 |
| Mean | −38.5 |
| Std Dev | 566.3 |
| Median | −244.5 |
| Min, Max | −769.0, 1093.0 |
| Maximum percentage change | |
| n | 10 |
| Mean | 27.3 |
| Std Dev | 156.59 |
| Median | −68.4 |
| Min, Max | −92.4, 357.2 |

Cutoff date: 14 Aug. 2023

TABLE 33
CD8+ T Cells in Peripheral Blood
of patients dosed with 16 mg/kg IgG1-C-E430G

| CD3+CD8+ ABS | Dose Level |
|---|---|
| (Cells/uL) | 16 mg/kg |
| Baseline | |
| n | 10 |
| Mean | 585.6 |
| Std Dev | 615.52 |
| Median | 362.5 |
| Min, Max | 101.0, 2210.0 |
| Maximum change | |
| n | 10 |
| Mean | 22.2 |
| Std Dev | 1071.34 |
| Median | −278 |
| Min, Max | −2067, 1788.0 |
| Maximum percentage change | |
| n | 10 |
| Mean | 204.3 |
| Std Dev | 573.62 |
| Median | −68.1 |
| Min, Max | −93.5, 1770.3 |

Cutoff date: 14 Aug. 2023

### Example 15—Evaluation of Complement Lytic Activity in Serum of Patients Dosed with 16 mg/Kg IgG1-C-E430G in Expansion Part A

### Methods

Total complement lytic activity (CH50) in serum from RRMM patients dosed with 16 mg/kg IgG1-C-E430G was evaluated as described in Example 7. As of the data cutoff date of 14 Aug. 2023, preliminary CH50 data were available from 10 subjects with RRMM dosed with 16 mg/kg IgG1-C-E430G in the Expansion Part A of the first-in-human trial.

Results: Complement Lytic Activity in Serum of Patients Dosed with IgG1-C-E430G

Table 34 show a transient decrease in total complement lytic activity (CH50) after administration of the first dose of IgG1-C-E430G. The median peak reduction from baseline (C1D1, pre-dose) was 60% (range −91%-40%, n=10). This suggests CDC activity of IgG1-C-E430G in the clinical setting. Complement parameters rapidly returned to baseline level in most subjects and remained at baseline levels after subsequent dosing, indicating that treatment does not exhaust complement.

In conclusion, transient decreases in complement lytic activity (CH50) were observed in the peripheral blood of patients dosed with IgG1-C-E430G, similar to observations in the Dose Escalation phase (Example 7). This confirms the biological activity of IgG1-C-E430G in vivo and is indicative of CDC activity of IgG1-C-E430G in patients.

TABLE 34
Complement lytic activity in serum of patients dosed with 16 mg/kg
lgG1-C-E430G

| Complement CH50 (U/mL) | $\frac{Dose Level}{16 mg / kg}$ |
|---|---|
| Baseline | |
| n | 10 |
| Mean | 49.9 |
| Std Dev | 10.39 |
| Median | 51 |
| Min, Max | 30.0, 60.0 |
| Maximum change | |
| n | 10 |
| Mean | −21.3 |
| Std Dev | 22.34 |
| Median | −28 |
| Min, Max | −48.0, 17.0 |
| Maximum percentage change | |
| n | 10 |
| Mean | −44 |
| Std Dev | 47.67 |
| Median | −59.5 |
| Min, Max | −90.6, 39.5 |

Cutoff date: 14 Aug. 2023

### Example 16—Evaluation of Cytokine Levels in Plasma of Patients Dosed with IgG1-C-E430G in Expansion Part A

### Methods

Cytokine levels in plasma from RRMM patients dosed with 16 mg/kg IgG1-C-E430G were evaluated as described in Example 8. As of the data cutoff date of 14 Aug. 2023, preliminary pharmacodynamic data were available from 11 subjects with RRMM dosed with 16 mg/kg IgG1-C-E430G in the Expansion Part A of the first-in-human trial.

### Results

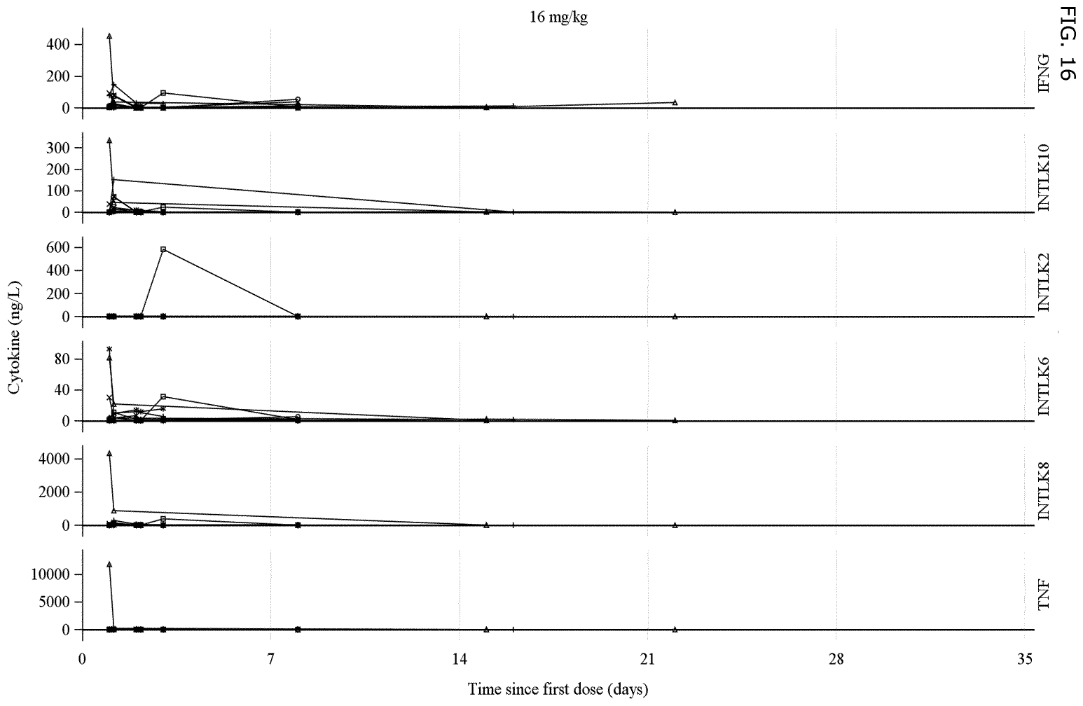

FIG. 16 and Table 35 shows no significant modulation of cytokine levels of IL-2, IL-6, IL-8, IL-10, IFNγ, and TNFα in plasma of patients dosed with 16 mg/kg IgG1-C-E430G, which is in line with observations in the dose escalation phase (Example 8).

TABLE 35
Cytokine levels in plasma of patients dosed with 16 mg/kg
IgG1-C-E430G (expansion part A, data cut off: 14 Aug. 2023)

| | | | | | | |
|---|---|---|---|---|---|---|
| | Interferon | Interleukin 2 | Interleukin 6 | Interleukin 8 | Interleukin 10 | Tumor Necrosis |
| Cytokine | Gamma (ng/L) | (ng/L) | (ng/L) | (ng/L) | (ng/L) | Factor (ng/L) |

Baseline

| | | | | | | |
|---|---|---|---|---|---|---|
| n | 11 | 11 | 11 | 11 | 11 | 11 |
| Mean | 62.7 | 1.4 | 19.8 | 403.9 | 34.4 | 1081.9 |
| Std | 133.18 | 0 | 34.56 | 1300.41 | 100.31 | 3557.5 |
| Dev | | | | | | |
| Median | 9.2 | 1.4 | 1.5 | 3.2 | 0.4 | 2.7 |
| Min, Max | 3.6, 453.0 | 1.4, 1.4 | 0.7, 92.9 | 2.1, 4324.0 | 0.4, 335.0 | 1.8, 11808 |

Maximum change

| | | | | | | |
|---|---|---|---|---|---|---|
| n | 11 | 11 | 11 | 11 | 11 | 11 |
| Mean | −23.3 | 53 | −13.6 | −305.4 | −2.9 | −1045 |
| Std | 150.01 | 175.67 | 36.56 | 1336.41 | 120.89 | 3568.83 |
| Dev | | | | | | |
| Median | 5.4 | 0 | 1.7 | 39 | 5.8 | 21.1 |
| Min, Max | −449.5, 90.0 | 0.0, 582.6 | −83.3, 30.7 | −4315, 383.9 | −333.4, 151.7 | −11805, 86.0 |

Maximum percentage change

| | | | | | | |
|---|---|---|---|---|---|---|
| n | 11 | 11 | 11 | 11 | 11 | 11 |
| Mean | 345.3 | 3838.1 | 464.8 | 3488.8 | 8508.7 | 1226.4 |
| Std | 660.6 | 12729.5 | 1319.67 | 5383.37 | 13914.9 | 1392.3 |
| Dev | | | | | | |
| Median | 68.3 | 0 | 159.3 | 842.6 | 1430.6 | 888 |
| Min, Max | −99.2, 1976.9 | 0.0, 42219 | −99.1, 4418.0 | −99.8, 18108 | −99.5, 43329 | −100.0, 3873.0 |

Cutoff date: 14 Aug. 2023

### Example 17—PK Evaluation in Expansion Part A

### Methods

The pharmacokinetics were evaluated for patients dosed with 16 mg/kg IgG1-C-E430G in the Expansion Part A of the first-in-human trial of IgG1-C-E430G.

As of the data cutoff date of 14 Aug. 2023, pharmacokinetic data were available from 11 subjects with RRMM dosed with IgG1-C-E430G from Expansion Part A and 23 subjects with RRMM dosed with IG-C-E430G from Dose Escalation.

Blood sampling schedules and assay methodology for determination of serum concentrations are similar to that utilized for Dose Escalation and is described in Example 9.

PK profiles included all serum concentrations from all PK-evaluable subjects. A value of 0 was imputed for all pretreatment concentrations and for predose concentrations below lower limit of quantification (BLQ) at the start of subsequent PK profiles.

### Results

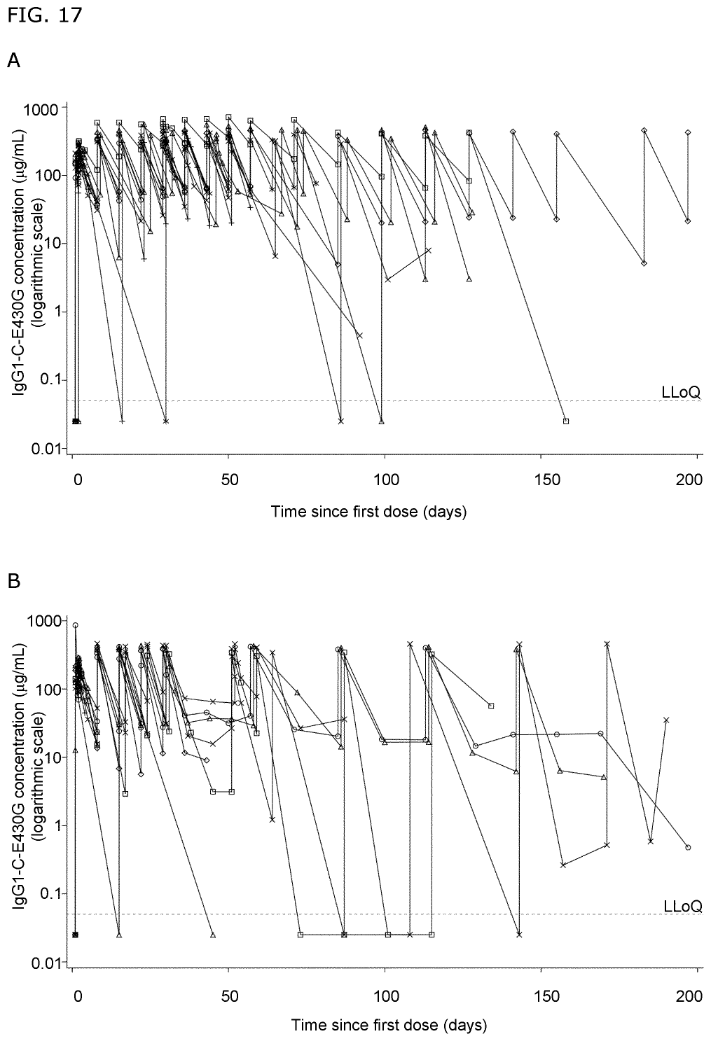

FIG. 17 shows the side by side comparison of the concentration time profile for subjects who received 16 mg/kg dose in Dose Escalation (A) and Expansion Cohort A (B). As expected, the PK profiles receiving the similar doses, is similar between the 2 cohorts. The peak and predose concentrations as observed in the figures are comparable between the cohorts.

1. A method of treating or preventing a hematological malignancy, in a subject in need thereof, comprising administering to said subject, an antibody binding to human CD38 in a therapeutically effective amount, said antibody comprising:
a. an antigen-binding region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4, a VL CDR1 having the sequence as set forth in SEQ ID NO:6, a VL CDR2 having the sequence AAS, and a VL CDR3 having the sequence as set forth in SEQ ID NO:7, and
b. an Fc region comprising a mutation in one or more amino acid residues selected from the group corresponding to E430, E345 and S440 in a human IgG1 heavy chain, wherein the amino acid residues are numbered according to the EU index.

2. The method of claim 1, wherein said antibody is administered at a dose of at least about 4 mg/kg body weight.

3. The method of claim 1, wherein said antibody is administered at a dose in the range of between about 4 mg/kg to about 24 mg/kg body weight.

4. The method of claim 1, wherein said antibody is administered at a dose in the range of between about 4 mg/kg to about 16 mg/kg body weight.

5. The method of claim 1, wherein said antibody is administered at a dose in the range of between about 4 mg/kg to about 8 mg/kg body weight.

6. The method of any one of claims 1-3, wherein said antibody is administered at a dose in the range of between about 8 mg/kg to about 16 mg/kg body weight.

7. The method of any one of claims 1-5, wherein said antibody is administered at a dose of about 4 mg/kg body weight.

8. The method of any one of claims 1-6, wherein said antibody is administered at a dose of about 8 mg/kg body weight.

9. The method of any one of claims 1-4 and 6, wherein said antibody is administered at a dose of about 16 mg/kg body weight.

10. The method of any one of claims 1-3, wherein said antibody is administered at a dose of about 24 mg/kg body weight.

11. The method of any one of claims 1-10, wherein said antibody is administered at a dose of about 250-2000 mg, such as about 280-1700 mg.

12. The method of any one of the preceding claims, wherein said antibody is administered in cycles of about 4 weeks or about 28 days, such as 4 weeks or 28 days.

13. The method of any one of the preceding claims, wherein said antibody is administered weekly (Q1W), preferably wherein said weekly administration is performed at least 8 times or for 2 cycles.

14. The method of any one of the preceding claims, wherein said antibody is administered once every two weeks (Q2W—biweekly), preferably wherein said administration every two weeks is performed at least 8 times or for 4 cycles, optionally wherein said administration every two weeks follows said weekly administration.

15. The method of any one of the preceding claims, wherein said antibody is administered once every four weeks (Q4W), preferably wherein said administration every four weeks is performed at least 1 time, optionally wherein said administration every 4 weeks follows an administration every week or every two weeks.

16. The method of any one of the preceding claims, wherein said antibody is administered in cycles of 28 days (4 weeks), with a weekly administration in cycles 1 and 2 (Q1W), a biweekly administration in cycles 3 through 6 (Q2W), and a monthly administration (Q4W) as of cycle 7 and onward.

17. The method of any one of the preceding claims, wherein said antibody is administered for a period of at least 2 cycles, preferably at least 4 cycles, more preferably at least 6 cycles, even more preferably at least 7 cycles.

18. The method of any one of the preceding claims, wherein at least the first dose of said antibody is administered as a split dose over two subsequent days, preferably split in about equal amounts.

19. The method of any one of the preceding claims, wherein said antibody is administered by intravenous injection or infusion.

20. The method of any one of the preceding claims, wherein said antibody is administered by intravenous injection or infusion in 100-500 ml over a period of 1 to 11 hours, such as 3 to 10 hours.

21. The method of any one of the preceding claims, wherein said hematological malignancy is a CD38 positive hematological malignancy or a hematological malignancy known to express CD38, and wherein said antibody is administered for a time sufficient to treat the CD38-positive hematological malignancy.

22. The method of any one of the preceding claims, wherein said hematological malignancy is a cancer that is relapsed or refractory to a prior anti-cancer therapy.

23. The method of any one of the preceding claims, wherein said hematological malignancy is a cancer that is refractory to a prior therapy comprising an anti-CD38 antibody.

24. The method of any one of claims 1-22, wherein said hematological malignancy is a cancer that is relapsed after a prior therapy comprising an antiCD38 antibody.

25. The method of claim 23 or 24, wherein the CD38 antibody is daratumumab or isatuximab.

26. The method of any one of claims 1-22, wherein said subject has not previously been treated with an aCD38 antibody.

27. The method of any one of claims 1-22 and 26, wherein said subject has not previously been treated with daratumumab and/or isatuximab.

28. The method of any one of the preceding claims, wherein said hematological malignancy is multiple myeloma (MM).

29. The method of any one of the preceding claims, wherein said hematological malignancy is relapsed or refractory multiple myeloma (RRMM).

30. The method of claim 29, wherein said relapsed or refractory multiple myeloma is characterized by evidence of disease progression in said subject on the most recent prior treatment regimen based on IMWG 2016 criteria with measurable disease, wherein said criteria are:
a. Prior documentation of monoclonal plasma cells in the bone marrow ≥10% or presence of a biopsy-proven plasmacytoma; and
b. Measurable disease at baseline as defined by:
i. IgG, IgA, IgD, or IgM myeloma: Serum M-protein level ≥0.5 g/dL (≥5 g/L) or urine M protein level ≥200 mg/24 hours; or
ii. Light chain myeloma: Serum Ig free light chain (FLC)≥10 mg/dL and abnormal serum Ig kappa lambda FLC ratio.

31. The method of any one of claims 1 to 27, wherein said hematological malignancy is diffuse large B-cell lymphoma (DLBCL), such as relapsed or refractory DLBCL.

32. The method of any one of the preceding claims, wherein the treatment induces one or more therapeutic effects in said subject, optionally wherein said on or more therapeutic effects is improved relative to a baseline.

33. The method of claim 32, wherein the one or more therapeutic effects is selected from the group consisting of: overall response rate, duration of response, time to response.

34. The method of claim 32 or 33, wherein the therapeutic effect is a stringent complete response, complete response, very good partial response, partial response, minimal response or stable disease status, and optionally can be continued until disease progression or lack of patient benefit.

35. The method of claim any one of the preceding claims, wherein said hematological malignancy preferably is (relapsed or refractory) multiple myeloma, wherein the therapeutic effect is an overall response rate of at least 14% in the treated subjects, optionally wherein said antibody is administered at a dose of at least (about) 4 mg/kg, such as between (about) 4 and 24 mg/kg.

36. The method of claim any one of claims 1-22, 26-34, wherein said hematological malignancy is a cancer, preferably multiple myeloma, that has not been previously treated with a prior therapy comprising an anti-CD38 antibody, such as daratumumab or isatuximab, wherein the therapeutic effect is an overall response rate of at least 40% in the treated subjects, optionally wherein said antibody is administered at a dose of at least (about) 4 mg/kg, such as between (about) 4 and 24 mg/kg.

37. The method of claim any one of claims 1-25, 28-34, wherein said hematological malignancy is a cancer that is relapsed or refractory to a prior anti-cancer therapy, such as a prior therapy comprising an anti-CD38 antibody, such as daratumumab or isatuximab, wherein the therapeutic effect is an overall/objective response rate of at least 6% in the treated subjects, optionally wherein said antibody is administered at a dose of (about) at a dose of at least (about) 4 mg/kg, such as between (about) 4 and 24 mg/kg, such at (about) 16 mg/kg.

38. The method of claim any one of claims 1-22, 26-34 and 36, wherein said hematological malignancy is a cancer, preferably multiple myeloma, that has not been previously treated with a prior therapy comprising an anti-CD38 antibody, preferably daratumumab or isatuximab, and wherein the therapeutic effect is at least 25%, such as at least 40% very good partial responses (VGPRs) or better in the treated subjects, such as at least 40% CR, optionally wherein said dose is at least about 4 mg/kg body weight or at least about 8 mg/kg body weight or at least about 16 mg/kg body weight or at least about 24/mg/kg body weight.

39. The method of claim any one of claims 1-25, 28-34 and 37, wherein said hematological malignancy is a cancer, preferably multiple myeloma, that is relapsed or refractory to a prior anti-cancer therapy, such as a prior therapy comprising an anti-CD38 antibody, preferably daratumumab or isatuximab, wherein the therapeutic effect is at least 6% partial responses in the treated subjects partial responses in the treated subjects, optionally wherein said dose is at least about 16 mg/kg body weight.

40. The method of any one of claims 32 to 39, wherein the one or more therapeutic effects is achieved at a dose of at least about 4 mg/kg body weight or at least about 8 mg/kg body weight or at least about 16 mg/kg body weight or at least 24/mg/kg body weight.

41. The method of any one of the preceding claims, wherein said subject is treated for the management of cytopenia, such as neutropenia or thrombocytopenia, e.g. grade 3 or grade 4 neutropenia or thrombocytopenia.

42. The method of any one of the preceding claims, wherein said subject is treated with granulocyte colony-stimulating factor (G-CSF).

43. The method of any one of the preceding claims, wherein said subject is treated for the management of infusion related reactions (IRRs), e.g. IRR of grade 2 or higher.

44. The method of any one of the preceding claims, wherein said subject is treated with pre-infusion medication before said administration of said antibody and/or with post-infusion medication after said administration of said antibody, optionally wherein said pre-infusion medication is administered about 1-3 hours prior to said administration of said antibody and/or wherein said post-infusion medication is administered on the two days following said administration of said antibody.

45. The method of claim 44, wherein said pre-infusion medication comprises corticosteroids (e.g methylprednisolone, betametasone, dexamethasone, triamcinolone, prednisone and/or prednisolone), antihistamines (e.g. diphenhydramine), antipyretics (e.g. paracetamol) and/or a leukotriene receptor antagonist (e.g. montelukast), optionally wherein
a. said corticosteroid is administered at a dose of about 60-100 mg methylprednisolone or equivalent;
b. Said diphenhydramine is administered at a dose of about 25-50 mg;
c. said paracetamol is administered at a dose of about 650-1000 mg; and/or
d. said montelukast is administered at a dose of about 10 mg 10 mg.

46. The method of claim 44 or 45, wherein said post-infusion medication comprises corticosteroids, e.g methylprednisolone, betametasone, dexamethasone, triamcinolone, prednisone and/or prednisolone, optionally wherein said corticosteroid is administered at a dose of 20 mg methylprednisolone or equivalent.

47. The method of any one of any one of the preceding claims, wherein said subject displays a faster clearance of said antibody compared to a reference antibody not comprising a mutation in one or more amino acid residues selected from the group corresponding to E430, E345 and S440 in a human IgG1 heavy chain, wherein the amino acid residues are numbered according to the EU index (when administered at a similar or comparable or equivalent dose).

48. The method of claim 47, wherein the antibody comprises a mutation at position E430, preferably E340G, and wherein said a reference antibody does not comprise a mutation at position E430 (i.e. is wt at said position), preferably wherein said reference antibody comprises a wildtype CH3/Fc region.

49. The method of claim 47 or 48, wherein said faster clearance occurs at a dose of at least 4 mg/kg body weight.

50. The method of any one of claims 47 to 49, wherein said clearance is defined as the dose divided by the estimated area under the serum or plasma concentration-time curve between start of administration and infinity.

51. The method of any one of claims 47 to 50, wherein said reference antibody is an IgG1 antibody not comprising a mutation in one or more amino acid residues selected from the group corresponding to E430, E345 and S440 in a human IgG1 heavy chain, wherein the amino acid residues are numbered according to the EU index, preferably comprising a wt CH3/Fc region.

52. The method of any on of claims 47 to 51, wherein said antibody comprises a mutation at position E430, preferably E430G, and wherein said reference antibody does not comprise said mutation at position E430 (is wt at said position), preferably wherein said antibody and said reference antibody, apart from any specified mutations, are IgG1 antibodies.

53. The method of any one of claims 47 to 52, wherein said reference antibody is daratumumab or isatuximab.

54. The method of any one of the preceding claims, wherein said antibody:
a. induces activation of the complement system in said subject;
b. induces depletion of peripheral blood NK cells in said subject; and/or
c. induces expansion of peripheral blood T cells in said subject.

55. The method of any one of the preceding claims, wherein said treatment induces activation of the complement system in said subject, optionally wherein said activation of the complement system is reflected by a (transient) reduction in complement component C2 and/or (transient) reduction in total complement lytic activity (CH50) in peripheral blood.

56. The method of claim 55, wherein said C2 levels are decreased by at least 30%, such as by at least (about) 35%, 40%, 45%, 50%, 55%, 58%, 60%, 64% from baseline and/or wherein said CH50 levels are decreased by at least 20%, such as by at least about 25%, 30%, 32%, 35%, 40%, 45%, 48%, 50%, 53%, 55%, or 60% from baseline.

57. The method of any one of claim 54 to 56, wherein said treatment induces activation of the complement system to a greater extent than a reference antibody not comprising a mutation in one or more amino acid residues selected from the group corresponding to E430, E345 and S440 in a human IgG1 heavy chain, wherein the amino acid residues are numbered according to the EU index (when administered at a similar or comparable dose), e.g. a reference antibody comprising a wildtype Fc region, optionally wherein said reference antibody is daratumumab.

58. The method of any one of claims 54 to 57, wherein activation/consumption of the complement system and/or said decrease in C2 and/or CH50 is transient, optionally returning to baseline within about 8 days.

59. The method of any one of the preceding claims, wherein said NK cell depletion is induced when said antibody is administered at a dose level of at least 0.2 mg/kg and remained during treatment.

60. The method of any one of claims 54 to 49, wherein T cell expansion is induced in a subject that has not received a prior therapy comprising a CD38 antibody, such as daratumumab or isatuximab, preferably daratumumab.

61. The method of any one of the preceding claims, wherein said treatment does not result in a substantial, dose dependent increase in plasma levels of proinflammatory cytokines, such as IL-2, IL-6, IL-8, IL-10, IFNγ and/or TNFα in said subject.

62. The method of any one of the preceding claims, wherein said treatment does not induce a dose-dependent reduction in B cells, T cells, monocytes and/or NKT-like cells, in said subject.

63. The method of any one of the preceding claims, wherein the antibody
a. has an inhibitory effect on CD38 cyclase activity in said subject;
b. induces complement-dependent cytotoxicity (CDC) of cells expressing human CD38 in said subject;
c. induces antibody-dependent cell-mediated cytotoxicity (ADCC) of cells expressing human CD38 in said subject;
d. induces antibody-dependent cellular phagocytosis (ADCP) of cells expressing human CD38 in said subject;
e. induces apoptosis in the presence of FcgR-bearing cells in said subject;
f. induces trogocytosis of cells expressing human CD38; or
g. any combination of a. to f.

64. The method of any one of the preceding claims, wherein said antibody induces trogocytosis-mediated reduction of CD38 on CD38-expressing tumor cells in said subject.

65. The method of any one of the preceding claims, wherein said antibody induces trogocytosis-mediated reduction of CD38 on CD38-expressing immune cells in said subject.

66. The method of claim 65, wherein the CD38-expressing immune cells are CD38-expressing immunosuppressive cells, preferably wherein the trogocytosis-mediated reduction of CD38 on the CD38-expressing immunosuppressive cells reduces their immunosuppressive activity.

67. The method of claim 66, wherein said CD38-expressing immunosuppressive cells comprise regulatory T cells (Tregs), regulatory B cells (Bregs), myeloid-derived suppressor cells (MDSCs), immunosuppressive NK cells, immunosuppressive NKT cells, immunosuppressive antigen-expressing cells (APCs), immunosuppressive macrophages, or any combination of two or more thereof, preferably Tregs.

68. The method of any one of claims 54 to 67, wherein any one or all of a. b and f. are higher compared to a reference antibody not comprising a mutation in one or more amino acid residues selected from the group corresponding to E430, E345 and S440 in a human IgG1 heavy chain, wherein the amino acid residues are numbered according to the EU index index (when administered at a similar or comparable or equivalent dose).

69. The method of claim 68, wherein said antibody comprises a mutation at position E430, preferably E430G, and wherein said reference antibody does not comprise said mutation at position E430 (is wt at said position), preferably wherein said antibody and said reference antibody, apart from any specified mutations, are IgG1 antibodies.

70. The method of claim 68 or 69, wherein said reference antibody is daratumumab or isatuximab.

71. The method of any one of the preceding claims, wherein said antibody comprises a variable heavy chain (VH) region comprising SEQ ID NO:1 or an amino acid sequence having at least 80% identity, such as 90%, or 95%, or 97%, or 98%, or 99%, to SEQ ID NO:1.

72. The method of any one of the preceding claims, wherein said antibody comprises a variable light chain (VL) region comprising SEQ ID NO:5 or an amino acid sequence having at least 80% identity, such as 90%, or 95%, or 97%, or 98%, or 99%, to SEQ ID NO:5.

73. The method of one of the preceding claims, wherein said antibody comprises a variable heavy (VH) region differing from SEQ ID NO:1 by 12 or less, such as 11, 10, 9, 8, 7, 6, 5, 4, 3, 2 or 1 mutations such as substitutions, insertions or deletions of amino acid residues.

74. The method of any one of the preceding claims, wherein said antibody comprises a variable light (VL) region differing from SEQ ID NO:5 by 12 or less, such as 11, 10, 9, 8, 7, 6, 5, 4, 3, 2 or 1 mutations such as substitutions, insertions or deletions of amino acid residues.

75. The method of any one of the preceding claims, wherein said antibody comprises a variable heavy (VH) region comprising the sequence of SEQ ID NO:1 and a variable light (VL) region comprising the sequence of SEQ ID NO:5.

76. The method of any one of the preceding claims, wherein the mutation in the one or more amino acid residues is selected from the group consisting of E430G, E345K, E430S, E430F, E430T, E345Q, E345R, E345Y, S440Y and S440W, preferably E430G, E345K, E430S and E345Q.

77. The method of any one of the preceding claims, wherein the mutation in the one or more amino acid residues comprises E430G.

78. The method of any one of the preceding claims, wherein the mutation in the one or more amino acid residues consists of E430G.

79. The method of any one of the preceding claims, wherein the Fc region comprises one or more further mutations which do not reduce complement-dependent cytotoxicity (CDC) and/or antibody-dependent cell-mediated cytotoxicity (ADCC) induced by the antibody variant without the one or more further mutations.

80. The method of claim 79, wherein the one or more further mutations are 12 or less, such as 11, 10, 9, 8, 7, 6, 5, 4, 3, 2 or 1 mutations such as substitutions, insertions or deletions of amino acid residues.

81. The method of any one of the preceding claims, wherein the Fc region is, except for the recited mutation, a human IgG1, IgG2, IgG3 or IgG4 isotype or a mixed isotype thereof.

82. The method of any one of the preceding claims, wherein the variant Fc region is, except for the recited mutation, a human IgG1 Fc region.

83. The method of any one of the preceding claims, wherein the Fc region is, except for the recited mutations, a human IgG1m(f), IgG1 m(a), IgG1m(x), IgG1m(z) allotype or a mixed allotype of any two or more thereof.

84. The method of any one of the preceding claims, wherein said antibody, except for the recited mutations, a human antibody.

85. The method of any one of the preceding claims, wherein said antibody is, except for the recited mutations, an IgG1 antibody.

86. The method of any one of the preceding claims, wherein said antibody is, except for the recited mutations, a human monoclonal full-length bivalent IgG1m(f), κ antibody.

87. The method of any one of the preceding claims, wherein CH region is a human IgG1 m(f), IgG1 m(a), IgG1 m(x) and IgG1 m(z) allotype, or a mixed allotype of any two or more thereof.

88. The method of any one of the preceding claims, wherein the CH region comprises, except for the recited mutations, the sequence of SEQ ID NO:19, SEQ ID NO:20, SEQ ID NO:21, SEQ ID NO:22, SEQ ID NO:23 or SEQ ID NO: 45.

89. The method of any one of the preceding claims, wherein the CH region comprises one or more further mutations.

90. The method of any one of the preceding claims, wherein Lys (K) at position 447 according to Eu numbering is deleted.

91. The method of any one of the preceding claims, wherein the CH region comprises an amino acid sequence selected from the group consisting of SEQ ID NO:24 to SEQ ID NO:33 and SEQ ID NO: 46.

92. The method of any one of the preceding claims, wherein the CH region comprises SEQ ID NO:24 or SEQ ID NO: 46, optionally wherein the light chain comprises a CL comprising SEQ ID NO:37.

93. The method of any one of the preceding claims, wherein the antibody is a bivalent antibody.

94. The method of any one of the preceding claims, wherein said antibody is a full-length antibody.

95. The method of any one of the preceding claims, wherein said antibody is a monoclonal antibody.

96. The method of any one of the preceding claims, wherein said antibody is a monospecific antibody.

97. The method of any one of claims 1 to 96, wherein said antibody is a bispecific antibody.

98. The method of any one of the preceding claims, wherein said antibody is comprised in a composition further comprising a pharmaceutically acceptable carrier.

99. The method of any one of the preceding claims, wherein said antibody is comprised in a composition comprising:
a) said antibody, optionally in a concentration of 1 to 200 mg/mL
b) 5-40 mM histidine or acetate;
c) 100-400 mM sorbitol or sucrose; and
d) a surfactant.

100. The method of any one of the preceding claims, wherein said antibody is comprised in a composition comprising having a pH of about 6 and comprising, consisting or consisting essentially of
a) about 20 mg/mL of the antibody,
b) about 20 mM histidine,
c) about 250 mM sorbitol, and
d) about 0.04% w/v of polysorbate 80,
optionally in aqueous solution.

101. The method of any one of the preceding claims, wherein said of the preceding claims, wherein said antibody is in a composition, which is a concentrate to be diluted; such as in a 0.9% NaCl (saline) or dextrose solution, optionally a 5% w/v dextrose solution.

102. The antibody as described in any of the preceding claims for use in the prevention or treatment of a hematological malignancy according to any one of claims 1 to 101.

103. Us of the antibody as described in of the preceding claims for the manufacture of a medicament for the prevention or treatment of a hematological malignancy according to any one of claims 1 to 101.


SEQUENCE LISTING (46 sequences)

SEQ ID NO: 1 - protein, 121 aa, synthetic construct
QVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSS

SEQ ID NO: 2 - protein, 8 aa, synthetic construct
GGTFSSYA

SEQ ID NO: 3 - protein, 8 aa, synthetic construct
IIRFLGIA

SEQ ID NO: 4 - protein, 14 aa, synthetic construct
AGEPGERDPDAVDI

SEQ ID NO: 5 - protein, 107 aa, synthetic construct
DIQMTQSPSSLSASVGDRVTITCRASQGIRSWLAWYQQKPEKAPKSLIYAASSLQSGVPSRFSGSGSGTDFTLTISSLQPEDFATYYCQQYNSYPLTFGGGTKVEIK

SEQ ID NO: 6 - protein, 6 aa, synthetic construct
QGIRSW

SEQ ID NO: 7 - protein, 9 aa, synthetic construct
QQYNSYPLT

SEQ ID NO: 8 - protein, 122 aa, synthetic construct [sequence omitted]

SEQ ID NO: 9 - protein, 107 aa, synthetic construct [sequence omitted]

SEQ ID NO: 10 - protein, 120 aa, synthetic construct
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSS

SEQ ID NO: 11 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 12 - protein, 127 aa, synthetic construct
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSS

SEQ ID NO: 13 - protein, 8 aa, synthetic construct
GYRFSNFV

SEQ ID NO: 14 - protein, 8 aa, synthetic construct
INPYNGNK

SEQ ID NO: 15 - protein, 20 aa, synthetic construct
ARVGPYSWDDSPQDNYYMDV

SEQ ID NO: 16 - protein, 108 aa, synthetic construct
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERK

SEQ ID NO: 17 - protein, 7 aa, synthetic construct
HSIRSRR

SEQ ID NO: 18 - protein, 9 aa, synthetic construct
QVYGASSYT

SEQ ID NO: 19 - protein, 330 aa, Homo sapiens [sequence omitted]

SEQ ID NO: 20 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKPVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKPVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 24 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 25 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHSALHNHYTQKSLSLSPGK

SEQ ID NO: 26 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHFALHNHYTQKSLSLSPGK

SEQ ID NO: 27 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHTALHNHYTQKSLSLSPGK

SEQ ID NO: 28 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRQPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRYPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKWLSLSPGK

SEQ ID NO: 33 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKYLSLSPGK

SEQ ID NO: 34 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 35 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 36 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 107 aa, Homo sapiens [sequence omitted]

SEQ ID NO: 38 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 39 - protein, 262 aa, synthetic construct
HHHHHHRWRQTWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 40 - protein, 8 aa, synthetic construct
  VARIANT 5: note = X is S or R
GGTFXSYA

SEQ ID NO: 41 - protein, 8 aa, synthetic construct
  VARIANT 3: note = X is R or V
  VARIANT 7: note = X is I or K
  VARIANT 8: note = X is A, T or V, such as A or T
IIXFLGXX

SEQ ID NO: 42 - protein, 14 aa, synthetic construct
  VARIANT 1: note = X is A or T
  VARIANT 6: note = X is E, D or A, such as E or D
  VARIANT 12: note = X is V or F
XGEPGXRDPDAXDI

SEQ ID NO: 43 - protein, 6 aa, synthetic construct
QGIRSW

SEQ ID NO: 44 - protein, 9 aa, synthetic construct
  VARIANT 5: note = S or N
QQYNXYPLT

SEQ ID NO: 45 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPG